(19) United States
(12) Patent Application Publication
(10) Pub. No.: US 2024/0059927 A1
(43) Pub. Date: Feb. 22, 2024

(54) VINYL ETHER-BASED INKJET INK PHOTOPOLYMERIZED BY THIOL-ENE CLICK CHEMISTRY USED FOR TOUGHENING OF PHOTOPOLYMERS

(71) Applicant: Align Technology, Inc., San Jose, CA (US)

(72) Inventors: Bettina KOCH, Mattersburg (AT); Timon THEUER, Purkersdorf (AT); Alexander HOCHWALLNER, Wien (AT); Robert LISKA, Schleinbach (AT); Jürgen STAMPFL, Vienna (AT); Yan CHEN, Cupertino, CA (US); Chunhua LI, Cupertino, CA (US); Peter DORFINGER, Los Altos Hills, CA (US)

(21) Appl. No.: 18/450,258

(22) Filed: Aug. 15, 2023

Related U.S. Application Data
(60) Provisional application No. 63/398,161, filed on Aug. 15, 2022.

(51) Int. Cl. C09D 129/10; C09D 11/102; C09D 11/107; A61C 7/00; A61C 7/08; B33Y 70/00; B33Y 80/00

(52) U.S. Cl. CPC C09D 129/10; C09D 11/102; C09D 11/107; A61C 7/002; A61C 7/08; B33Y 70/00; B33Y 80/00; A61C 2201/00; B29C 64/124

(57) ABSTRACT

The present disclosure provides a range of printed materials comprising discrete layers or segment with distinct compositions, and which can collectively lend to high levels of strength, toughness, and break resistance, and these printed materials may contain thin, ductile layers interspersed between thicker, harder layers, thus in printing such materials, the present disclosure further provides a range of compositions and methods, including low viscosity, air stable, and rapidly solutions amenable to thin layer inkjet printing.

### INCORPORATION BY REFERENCE

All publications, patents, and patent applications mentioned in this specification are herein incorporated by reference to the same extent as if each individual publication, patent, or patent application was specifically and individually indicated to be incorporated by reference. This application claims priority to U.S. Provisional Patent Application No. 63/398,161, filed Aug. 15, 2022, entitled “VINYL ETHER-BASED INKJET INK PHOTOPOLYMERIZED BY THIOL-ENE CLICK CHEMISTRY USED FOR TOUGHENING OF PHOTOPOLYMERS,” the content of which is hereby incorporated by reference as if set forth fully herein.

### BACKGROUND

As there is often a tradeoff between toughness and resiliency, photopolymers generated by additive manufacturing technologies are often suffer from low toughness or high brittleness, rendering them unsuitable for wide ranges of applications. While many approaches to material engineering focus on molecular-level optimizations, many applications require combinations of material properties not attainable with individual compositions. Accordingly, strategies for enhancing device properties beyond the limitations of constituent parts are not only needed to meet present demands, but also to enable next-generation technologies.

### SUMMARY

Accordingly, the present disclosure is directed to a vinyl ether-based inkjet ink photopolymerized by thiol-ene click chemistry used for toughening photopolymers that substantially obviates one or more of problems due to limitations and disadvantages described above.

Additional features and advantages of the disclosure will be set forth in the description which follows and in part will be apparent from the description, or may be learned by practice of the disclosure. Other advantages of the present disclosure will be realized and attained by the structure particularly pointed out in the written description and claims hereof as well as the appended drawings.

To achieve these and other advantages and in accordance with the present disclosure, as embodied and broadly described a method of producing a multilayered article comprises producing a first layer of the multilayered article by photolithography from a matrix material; and producing a second layer of the multilayered article on the first layer by inkjet printing using an ink through a thiol-ene reaction.

In another as aspect of the present disclosure, a method of producing a multilayered article comprises curing a first portion of a matrix material having a first composition to form a first layer of the multilayered article; applying an ink that is stable for at least 20 days at 25° C. having a second composition to the first layer of the multilayered article; and curing the ink, thereby forming a second layer disposed on the first layer of the multilayered article.

In another aspect of the present disclosure, a photocurable composition comprises a first difunctional monomer comprising two ethylenically unsaturated functional groups; a second difunctional monomer comprising two thiol functional groups; and a crosslinker comprising at least three thiol or at least three ethylenically unsaturated functional groups, wherein a molar ratio of ethylenically unsaturated functional groups and thiol groups is between 2:5 to 5:2.

In another aspect of the present disclosure, a digital multilayered article comprises a first layer comprising an acrylate or a methacrylate; and a second layer chemically bonded to the first layer and cured from an ink material selected from the group consisting of tri(ethylene glycol) divinyl ether (TEGDVE), 2,2′-(ethylenedioxy diethanethiol (EDDT), a crosslinking agent comprising 1,1,1-tris-(hydroxymethyl)-propane-tris-(3-mercaptopropionate) TMPMP, or any combination thereof.

In another aspect of the present disclosure, a digital multilayered article comprises a matrix layer comprising an acrylate or a methacrylate; and an ink layer chemically bonded to the matrix layer and cured from an ink material comprising 50 fg % vinyl ether tri(ethylene glycol) divinyl ether (TEGDVE), between 30 and 45 fg % difunctional thiol 2,2′-(ethylenedioxy) diethanoethiol (EDDT) and between 5 and 20 fg % trifunctional thiol 1,1,1-tris-(hydroxymethyl)-propane-tris-(3-mercaptopropionate) (TMPMP) and 1 wt % cellulose acetate butyrate (CAB).

In another aspect of the present disclosure, a medical device comprises a cured first composition; and a cured second composition coating at least a portion of the cured first composition, wherein a thickness of the cured second composition is at most 300 microns (μm).

In a further aspect of the present disclosure, A method of repositioning a patient's teeth, the method comprises generating a treatment plan for the patient, the plan comprising a plurality of intermediate tooth arrangements for moving teeth along a treatment path from an initial tooth arrangement toward a final tooth arrangement; producing a dental appliance comprising the multilayered article described above; and moving on-track, with the dental appliance, at least one of the patient's teeth toward an intermediate tooth arrangement or the final tooth arrangement.

It is to be understood that both the foregoing general description and the following detailed description are exemplary and explanatory and are intended to provide further explanation of the disclosure as claimed.

### DESCRIPTION OF THE DRAWINGS

The novel features of the invention are set forth with particularity in the appended claims. This patent or application file contains at least one drawing executed in color. Copies of this patent or patent application publication with color drawings will be provided by the U.S. Patent and Trademark Office upon request and payment of the necessary fee. A better understanding of the features and advantages of the present disclosure will be obtained by reference to the following detailed description that sets forth illustrative embodiments, in which the principles of the invention are utilized, and the accompanying drawings of which:

FIG. 1A illustrates a tooth repositioning appliance, in accordance with embodiments.

FIG. 1B illustrates a tooth repositioning system, in accordance with embodiments.

FIG. 1C illustrates a method of orthodontic treatment using a plurality of appliances, in accordance with embodiments.

FIG. 2 illustrates a method for designing an orthodontic appliance, in accordance with embodiments.

FIG. 3 illustrates a method for digitally planning an orthodontic treatment, in accordance with embodiments.

FIG. 4 provides chemical structures of thiol and ene monomers consistent with the present disclosure.

FIG. 5 outlines viscosity changes of a curable composition during refrigerated and 50° C. storage. PyG90_50 C=40 mPa*s at less than 10 d; PyG90_fridge=approximately 10 mPa*s at 80 d.

FIG. 6 provides viscosity changes of a curable composition during storage at multiple temperatures. From bottom to top: PyG90_fridge; PyG90_25° C.; PyG9037° C.; PyG90_50° C.

FIG. 7 provides viscosity changes of a curable composition during storage at multiple temperatures. From bottom to top: PyG180_fridge; PyG180_25° C.; PyG180_37° C.; PyG180_50° C.

FIG. 8 provides results of Dynamic Mechanical Thermal Analysis (DMTA) on a curable composition, with storage modulus G′ shown with the solid plot and on the leftmost axis, and the dissipation factor (the tangent of the phase angle (δ) between stress and strain, tanδ) shown with the dotted plot and on the rightmost axis.

FIG. 9 provides results of stress-strain analyses on a cured composition.

FIG. 10 provides results of elastic modulus analyses on a cured composition.

FIG. 11 provides results of elastic modulus analyses on a cured composition.

FIG. 12 provides results of elastic modulus analyses on a cured composition.

FIG. 13 provides results of elastic modulus analyses on a cured composition.

FIG. 14 summarizes results of tensile tests on a pure matrix material and on digital materials with different curable composition geometries. From bottom to top of the right side of the figure: Layers; Lines; Matrix; Dots.

FIG. 15 provides laser scanning microscopy (LSM) images of a printed curable composition.

FIG. 16 provides viscosity changes of a curable composition during storage at multiple temperatures. From bottom to top at 20 d: VET20_fridge; VET20_37° C.; VET20_25° C.; VET20_50° C.

FIG. 17 provides viscosity changes of two curable compositions during storage at multiple temperatures. From bottom to top at 20 d: VET10_fridge; VET10_37° C.; VET10_25° C.; VET10_50° C.; VET10_1CAB_fridge; VET10_1CAB_37° C.; VET10_1CAB_50° C.; VET10_1CAB_25° C.

FIG. 18 provides viscosity changes of two curable compositions during storage at multiple temperatures. From bottom to top at 20 d: VET5_fridge; VET5_37° C.; VET5_25° C.; VET5_50° C.; VET5_1CAB_fridge; VET5_1CAB_37° C.; VET5_1CAB_25° C.; VET5_1CAB50° C.

FIG. 19 summarizes double bond conversion (DBC) rates determined with attenuated total reflectance infrared spectroscopy (ATR-IR) for 30 μm layers of multiple cured compositions.

FIG. 20 provides polymerization rates (R_{P}) for multiple curable compositions.

FIG. 21 provides times required to reach 95% photopolymerization (t 95%) for multiple curable compositions.

FIG. 22 provides double bond conversion rates at the gel points (t_{G}) for multiple curable compositions.

FIG. 23 displays time to reach gel point (t_{G}) for multiple curable compositions.

FIG. 24 provides storage modulus G′ and the dissipation factor tanδ as functions of temperature for multiple curable compositions. From top to bottom at −100 C for solid lines: VET5; VET5_1CAB; VET20; VET10_1CAB; VET10. From top to bottom at peak maxima for dotted lines: VET5; VET5_1CAB; VET10; VET10_1CAB; VET20.

FIG. 25 displays a stress strain curve for multiple curable compositions. From bottom to top at 0% strain: Vet20; VET10; VET10_1CAB; VET5_1CAB; VET5.

FIG. 26 provides laser scanning microscopy (LSM) images of multiple curable compositions printed with different waveforms.

FIG. 27 provides results of tensile test curves of a matrix and multiple digital materials. From bottom to top at 0% strain: Dots; Lines; Layers; Matrix.

FIG. 28 summarizes results of tensile tests on a pure matrix material and on digital materials with different curable composition geometries. From bottom to top at 5% strain: Matrix; Dots; Lines; Layers.

FIG. 29 provides LSM images of the digital materials of FIG. 28.

FIG. 30 summarizes results of tensile tests on a pure matrix material and on digital materials with different curable composition geometries. From bottom to top at 5% strain: Matrix; Lines; Dots; Layers.

FIG. 31 provides results of elastic modulus analyses on the digital material of FIG. 30 with line geometry.

FIG. 32 provides LSM images of the digital materials of FIG. 30.

FIG. 33 summarizes results of tensile tests on a pure matrix material and on digital materials with different curable composition geometries. From bottom to top at 5% strain: Matrix; Dots; Lines; Layers.

FIG. 34 provides LSM images of the digital materials of FIG. 33.

### DETAILED DESCRIPTION

Disclosed herein are digital materials with enhanced physical and mechanical properties, as well as compositions and methods for generating them. Conventional materials are often subject to a tradeoff between strength and toughness, such that strong materials typically suffer from brittleness, while pliable materials often lack strength and hardness. Overcoming these limitations, digital materials of the present disclosure can comprise the advantageous properties of their constituent components, thereby exhibiting enhanced characteristics beyond what would be expected from the sum of individual segments.

A digital material of the present disclosure can comprise discrete layers or segments, each of which can comprise distinct physical and chemical properties. The distinct segments can provide complementary, additive effects, thereby mitigating deficient properties of individual segments. In many cases, the digital material comprises alternating layers of hard and ductile segments or layers. When subject to stress, the hard regions can provide resistance to deformation, warping, and failure, while the ductile regions can elastically accommodate the stress to prevent breaking and crack propagation. Furthermore, over the lifespan of such materials, the ductile regions can limit the spread of breaks and deformations within hard regions, thereby confining damage to individual hard regions and delaying device failure. In order to achieve high levels of toughness and fine detail, the digital materials disclosed herein often include thin layers of ductile materials less than 100 microns (μm) thick, not readily printable with conventional compositions.

In some aspects, the present disclosure provides a method for producing a multilayered article, the method comprising: producing a first layer of the multilayered article by photolithography from a matrix; and producing a second layer of the multilayered article by inkjet printing using a ink that is stable for at least 20 days at 25° C., thereby producing a multilayered article.

In some aspects, the present disclosure provides a method of producing a multilayered article, the method comprising: curing a first portion of a matrix to form a first layer of the multilayered article; separating the first layer of the multilayered article from a second portion of the matrix not cured; applying an ink that is stable for at least 20 days at 25° C. to the first layer of the multilayered article; and curing the ink, thereby forming a second layer disposed on the first layer of the multilayered article.

As a non-limiting means for generating thin layers in a spatially controlled manner (e.g., thin layers of the ink), the present disclosure provides inkjet printing methods for applying low viscosity compositions. Following generation of a hard substrate (e.g., a photolithographically generated layer of the matrix), inkjet printing can be used to selectively apply thin layers of the second composition with controlled thickness. The combination of low viscosity and thickness of the inkjet-applied layers can enable high degrees of conformity to underlying structure, allowing fine features from the hard substrate to be retained through intervening ductile layer applications.

Further aspects of the present disclosure provide the matrix and inks suitable for digital device printing, as well as the digital devices printed therefrom. As the material requirements can differ between digital material segments or layers, many digital materials disclosed herein are printed with two or more curable compositions. As an example, a digital material with alternating hard and ductile layers can utilize a matrix comprising a high-crosslinking acrylate-urethane composition for hard layer printing and a low cross-linking thiolene ink for soft layer printing. A digital material can be printed from at least two, at least three, at least four, or at least five or more curable compositions (e.g., the first and second compositions along with additional third, fourth, and/or fifth compositions). Alternatively, a digital material can be printed from a single composition variably treated or cured to generate discrete regions with distinct properties.

As described further herein, the matrix and inks can comprise a plurality of components, including one or more species of polymerizable compounds of the present disclosure (e.g., 1, 2, 3, or more different species); one or more species of polymerizable monomers (e.g., reactive diluents); one or more solid materials (e.g., a nanoparticle or microparticle); one or more photoinitiators; one or more toughness, glass transition temperature, or viscosity modifiers; as well as combinations thereof. The curable compositions can be a photo-curable, a thermo-curable, a chemically curable, or a combination thereof. In many cases, the curable compositions are photo-curable.

### I. Definitions

All terms, chemical names, expressions and designations have their usual meanings which are well-known to those skilled in the art. As used herein, the terms “to comprise” and “comprising” are to be understood as non-limiting, i.e., other components than those explicitly named may be included.

Number ranges are to be understood as inclusive, i.e., including the indicated lower and upper limits. Furthermore, the term “about”, as used herein, and unless clearly indicated otherwise, generally refers to and encompasses plus or minus 10% of the indicated numerical value(s). For example, “about 10%” may indicate a range of 9% to 11%, and “about 1” may include the range 0.9-1.1.

As used herein, the term “polymer” generally refers to a molecule composed of repeating structural units connected by covalent chemical bonds and characterized by a substantial number of repeating units (e.g., equal to or greater than 20 repeating units and often equal to or greater than 100 repeating units and often equal to or greater than 200 repeating units) and a molecular weight greater than or equal to 5,000 Daltons (Da) or 5 kDa, such as greater than or equal to 10 kDa, 15 kDa, 20 kDa, 30 kDa, 40 kDa, 50 kDa, or 100 kDa. Polymers are commonly the polymerization product of one or more monomer precursors. The term polymer includes homopolymers, i.e., polymers consisting essentially of a single repeating monomer species. The term polymer also includes copolymers which are formed when two or more different types (or species) of monomers are linked in the same polymer. Copolymers may comprise two or more different monomer species, and include random, block, alternating, segmented, grafted, tapered and other copolymers. The term “cross-linked polymers” generally refers to polymers having one or multiple links between at least two polymer chains, which can result from multivalent monomers forming cross-linking sites upon polymerization.

As used herein, the term “oligomer” generally refers to a molecule composed of repeating structural units connected by covalent chemical bonds and characterized by a number of repeating units less than that of a polymer (e.g., equal to or less than 20 or less than 10 repeating units) and a lower molecular weight than polymers, e.g., less than 5,000 Da or less than 2,000 Da, and in various cases from about 0.5 kDa to about 5 kDa. In some cases, oligomers may be the polymerization product of one or more monomer precursors.

As used herein, the term “reactive diluent” generally refers to a substance which reduces the viscosity of another substance, such as a monomer or curable composition. A reactive diluent may become part of another substance, such as a polymer obtained by a polymerization process. In some examples, a reactive diluent is a curable monomer which, when mixed with a curable composition, reduces the viscosity of the resultant formulation and is incorporated into the polymer that results from polymerization of the formulation.

Oligomer and polymer mixtures can be characterized and differentiated from other mixtures of oligomers and polymers by measurements of molecular weight and molecular weight distributions.

The average molecular weight (M) is the average number of repeating units n times the molecular weight or molar mass (Mi) of the repeating unit. The number-average molecular weight (Mn) is the arithmetic mean, representing the total weight of the molecules present divided by the total number of molecules.

The term “biocompatible,” as used herein, refers to a material that does not elicit an immunological rejection or detrimental effect, referred herein as an adverse immune response, when it is disposed within an in-vivo biological environment. For example, in embodiments a biological marker indicative of an immune response changes less than 10%, or less than 20%, or less than 25%, or less than 40%, or less than 50% from a baseline value when a human or animal is exposed to or in contact with the biocompatible material. Alternatively, immune response may be determined histologically, wherein localized immune response is assessed by visually assessing markers, including immune cells or markers that are involved in the immune response pathway, in and adjacent to the material. In an aspect, a biocompatible material or device does not observably change immune response as determined histologically. In some embodiments, the disclosure provides biocompatible devices configured for long-term use, such as on the order of weeks to months, without invoking an adverse immune response. Biological effects may be initially evaluated by measurement of cytotoxicity, sensitization, irritation and intracutaneous reactivity, acute systemic toxicity, pyrogenicity, subacute/subchronic toxicity and/or implantation. Biological tests for supplemental evaluation include testing for chronic toxicity.

“Bioinert” refers to a material that does not elicit an immune response from a human or animal when it is disposed within an in-vivo biological environment. For example, a biological marker indicative of an immune response remains substantially constant (plus or minus 5% of a baseline value) when a human or animal is exposed to or in contact with the bioinert material. In some embodiments, the disclosure provides bioinert devices.

As used herein, the terms “homogenous” and “homogeneity” may refer to uniformity in a constituent distribution or a property of a composition or material. In some cases, homogenous denotes distributional uniformity at the microscopic, nanoscopic, or chemical level. In some cases, homogenous refers to random distributions of components within a composition or material. In some cases, homogenous denotes spatially invariant chemical or physical properties (e.g., spatially uniform hardness or color) of a composition or material.

As used herein, the terms “heterogenous” and “heterogeneity” may refer to nonuniformity in a constituent distribution or a property of a composition or material. In some cases, “heterogenous” denotes partitioning or patterning within a composition or material. For example, an emulsion may be heterogenous to the degree that two or more components separate into distinct phases. In some cases, heterogenous denotes spatial variance in chemical or physical properties of a composition or material. In some cases, heterogenous denotes nonuniform crystallinity over a portion of a composition or material.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individually or in any combination. Additionally, unless otherwise specified, all isotopic variants of compounds disclosed herein are intended to be encompassed by the disclosure. Specific names of compounds are intended to be exemplary, as it is known that one of ordinary skill in the art can name the same compounds differently.

It is noted that as used herein and in the appended claims, the singular forms “a”, “an”, and “the” include plural reference unless the context clearly dictates otherwise. Thus, for example, reference to “a monomer” includes a plurality of such monomers and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim.

As used herein, the term “group” may refer to a reactive functional group of a chemical compound. Groups of the present compounds refer to an atom or a collection of atoms that are a part of the compound. Groups of the present disclosure may be attached to other atoms of the compound via one or more covalent bonds. Groups may also be characterized with respect to their valence state. The present disclosure includes groups characterized as monovalent, divalent, trivalent, etc. valence states.

As used herein, the term “substituted” refers to a compound (e.g., an alkyl chain) wherein a hydrogen is replaced by another reactive functional group or atom, as described herein.

As used herein, a broken line in a chemical structure can be used to indicate a bond to the rest of the molecule. For example, [symbol] in

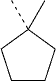

is used to designate the 1-position as the point of attachment of 1-methylcyclopentate to the rest of the molecule. Alternatively,

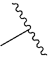

in, e.g.,

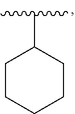 [SMILES: *C1CCCCC1]

can be used to indicate that the given moiety, the cyclohexyl moiety in this example, is attached to a molecule via the bond that is “capped” with the wavy line.

Alkyl groups include straight-chain, branched and cyclic alkyl groups, unless otherwise defined for a compound or genus of compounds. Alkyl groups include those having from 1 to 30 carbon atoms, unless otherwise defined. Thus, alkyl groups can include small alkyl groups having 1 to 3 carbon atoms, medium length alkyl groups having from 4-10 carbon atoms, as well as long alkyl groups having more than 10 carbon atoms, particularly those having 10-30 carbon atoms. The term cycloalkyl specifically refers to an alkyl group having a ring structure such as a ring structure comprising 3-30 carbon atoms, optionally 3-20 carbon atoms and optionally 3-10 carbon atoms, including an alkyl group having one or more rings. Cycloalkyl groups include those having a 3-, 4-, 5-, 6-, 7-, 8-, 9- or 10- member carbon ring(s) and particularly those having a 3-, 4-, 5-, 6-, 7- or 8-member ring(s). The carbon rings in cycloalkyl groups can also carry alkyl groups. Cycloalkyl groups can include bicyclic and tricyclic alkyl groups. Alkyl groups are optionally substituted, as described herein. Substituted alkyl groups can include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Unless otherwise defined herein, substituted alkyl groups include fully halogenated or semihalogenated alkyl groups, such as alkyl groups having one or more hydrogens replaced with one or more fluorine atoms, chlorine atoms, bromine atoms and/or iodine atoms. Thus, substituted alkyl groups can include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alkyl portion of the groups is substituted as provided herein in connection with the description of alkyl groups. As used herein MeO—refers to CH₃O—. Moreover, a thioalkoxy group, as used herein is an alkyl group that has been modified by linkage to sulfur atom (instead of an oxygen) and can be represented by the formula R—S.

Alkenyl groups include straight-chain, branched and cyclic alkenyl groups. Alkenyl groups include those having 1, 2 or more double bonds and those in which two or more of the double bonds are conjugated double bonds. Unless otherwise defined herein, alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6-, 7- or 8-member ring(s). The carbon rings in cycloalkenyl groups can also carry alkyl groups. Cycloalkenyl groups can include bicyclic and tricyclic alkenyl groups. Alkenyl groups are optionally substituted. Unless otherwise defined herein, substituted alkenyl groups include among others those that are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups can include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms.

Aryl groups include groups having one or more 5-, 6-, 7- or 8-membered aromatic rings, including heterocyclic aromatic rings. The term heteroaryl specifically refers to aryl groups having at least one 5-, 6-, 7- or 8-member heterocyclic aromatic ring. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those that are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein provided in a covalently bonded configuration in the compounds of the disclosure at any suitable point of attachment. In some embodiments, aryl groups contain between 5 and 30 carbon atoms. In some embodiments, aryl groups contain one aromatic or heteroaromatic six-member ring and one or more additional five- or six-member aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents.

Arylalkyl groups are alkyl groups substituted with one or more aryl groups wherein the alkyl groups optionally carry additional substituents and the aryl groups are optionally substituted. Specific arylalkyl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms.

As used herein, the terms “alkylene” and “alkylene group” are used synonymously and refer to a divalent group “—CH₂—” derived from an alkyl group as defined herein. The disclosure includes compounds having one or more alkylene groups. Alkylene groups in some compounds function as attaching and/or spacer groups. Compounds of the disclosure may have substituted and/or unsubstituted C₁-C₂₀ alkylene, C₁-C₁₀ alkylene and C₁-C₆ alkylene groups.

As used herein, the terms “cycloalkylene” and “cycloalkylene group” are used synonymously and refer to a divalent group derived from a cycloalkyl group as defined herein. The disclosure includes compounds having one or more cycloalkylene groups. Cycloalkylene groups in some compounds function as attaching and/or spacer groups. Compounds of the disclosure may have substituted and/or unsubstituted C₃-C₂₀ cycloalkylene, C₃-C₁₀ cycloalkylene and C₃-C₅ cycloalkylene groups.

As used herein, the terms “arylene” and “arylene group” are used synonymously and refer to a divalent group derived from an aryl group as defined herein. The disclosure includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as attaching and/or spacer groups. Arylene groups in some compounds function as chromophore, fluorophore, aromatic antenna, dye and/or imaging groups. Compounds of the disclosure include substituted and/or unsubstituted C₅-C₃₀ arylene, C₅-C₂₀ arylene, C₅-C₁₀ arylene and C₅-C₈ arylene groups.

As used herein, the terms “heteroarylene” and “heteroarylene group” are used synonymously and refer to a divalent group derived from a heteroaryl group as defined herein. The disclosure includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as attaching and/or spacer groups. Heteroarylene groups in some compounds function as chromophore, aromatic antenna, fluorophore, dye and/or imaging groups. Compounds of the disclosure include substituted and/or unsubstituted C₅-C₃₀ heteroarylene, C₅-C₂₀ heteroarylene, C₅-C₁₀ heteroarylene and C₅-C₈ heteroarylene groups.

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as attaching and/or spacer groups. Compounds of the disclosure include substituted and/or unsubstituted C₂-C₂₀ alkenylene, C₂-C₁₀ alkenylene and C₂-C₅ alkenylene groups.

As used herein, the terms “cycloalkenylene” and “cycloalkenylene group” are used synonymously and refer to a divalent group derived from a cycloalkenyl group as defined herein. The disclosure includes compounds having one or more cycloalkenylene groups. Cycloalkenylene groups in some compounds function as attaching and/or spacer groups. Compounds of the disclosure include substituted and/or unsubstituted C₃-C₂₀ cycloalkenylene, C₃-C₁₀ cycloalkenylene and C₃-C₅ cycloalkenylene groups.

As used herein, the terms “alkynylene” and “alkynylene group” are used synonymously and refer to a divalent group derived from an alkynyl group as defined herein. The disclosure includes compounds having one or more alkynylene groups. Alkynylene groups in some compounds function as attaching and/or spacer groups. Compounds of the disclosure include substituted and/or unsubstituted C₂-C₂₀ alkynylene, C₂-C₁₀ alkynylene and C₂-C₅ alkynylene groups.

As used herein, the terms “halo” and “halogen” can be used interchangeably and refer to a halogen group such as a fluoro (—F), chloro (—Cl), bromo (—Br) or iodo (—I).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and reactive functional groups, for example, provided as substituents.

The term “carbocyclic” refers to ring structures containing only carbon atoms in the ring. Carbon atoms of carbocyclic rings can be bonded to a wide range of other atoms and reactive functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a sub stituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

The term “heteroalkyl”, as used herein, generally refers to an alkyl, alkenyl or alkynyl group as defined herein, wherein at least one carbon atom of the alkyl group is replaced with a heteroatom. In some instances, heteroalkyl groups may contain from 1 to 18 non-hydrogen atoms (carbon and heteroatoms) in the chain, or from 1 to 12 non-hydrogen atoms, or from 1 to 6 non-hydrogen atoms, or from 1 to 4 non-hydrogen atoms. Heteroalkyl groups may be straight or branched, and saturated or unsaturated. Unsaturated heteroalkyl groups have one or more double bonds and/or one or more triple bonds. Heteroalkyl groups may be unsubstituted or substituted. Exemplary heteroalkyl groups include, but are not limited to, alkoxyalkyl (e.g., methoxymethyl), and aminoalkyl (e.g., alkylaminoalkyl and dialkylaminoalkyl). Heteroalkyl groups may be optionally substituted with one or more substituents.

The term “carbonyl”, as used herein, for example in the context of C₁₋₆ carbonyl substituents, generally refers to a carbon chain of given length (e.g., C₁₋₆), wherein each of the carbon atom of a given carbon chain can form the carbonyl bond, as long as it chemically feasible in terms of the valence state of that carbon atom. Thus, in some instance, the “C₁₋₆ carbonyl” substituent refers to a carbon chain of between 1 and 6 carbon atoms, and either the terminal carbon contains the carbonyl functionality, or an inner carbon contains the carbonyl functionality, in which case the substituent could be described as a ketone. The term “carboxy”, as used herein, for example in the context of C₁₋₆ carboxyl substituents, generally refers to a carbon chain of given length (e.g, C₁₋₆), wherein a terminal carbon contains the carboxy functionality, unless otherwise defined herein.

As to any of the groups described herein that contain one or more substituents, it is understood that such groups do not contain any substitution or substitution patterns which are sterically impractical and/or synthetically non-feasible. In addition, the compounds of this disclosure include all stereochemical isomers arising from the substitution of these compounds.

Unless otherwise defined herein, optional substituents for any alkyl, alkenyl and aryl group includes substitution with one or more of the following substituents, among others:
halogen, including fluorine, chlorine, bromine or iodine;
   pseudohalides, including —CN, —OCN (cyanate), —NCO (isocyanate), —SCN (thiocyanate) and —NCS (isothiocyanate);
   —COOR, where R is a hydrogen or an alkyl group or an aryl group and more specifically where R is a methyl, ethyl, propyl, butyl, or phenyl group all of which groups are optionally substituted;
   —COR, where R is a hydrogen or an alkyl group or an aryl group and more specifically where R is a methyl, ethyl, propyl, butyl, or phenyl group all of which groups are optionally substituted;
   —CON(R)₂, where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
   —OCON(R)₂, where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
   —N(R)₂, where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring that can contain one or more double bonds and can contain one or more additional carbon atoms;
   —SR, where R is hydrogen or an alkyl group or an aryl group and more specifically where R is hydrogen, methyl, ethyl, propyl, butyl, or a phenyl group, which are optionally substituted;
   —SO₂ R, or —SOR, where R is an alkyl group or an aryl group and more specifically where R is a methyl, ethyl, propyl, butyl, or phenyl group, all of which are optionally substituted;
   —OCOOR, where R is an alkyl group or an aryl group;
   —SO₂N(R)₂, where each R, independently of each other R, is a hydrogen, or an alkyl group, or an aryl group all of which are optionally substituted and wherein R and R can form a ring that can contain one or more double bonds and can contain one or more additional carbon atoms; and

—OR, where R is H, an alkyl group, an aryl group, or an acyl group all of which are optionally substituted. In a particular example R can be an acyl yielding —OCOR″, wherein R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri-, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups that contain one or more substituents, it is understood that such groups do not contain any substitution or substitution patterns which are sterically impractical and/or synthetically non-feasible. In addition, as further described herein, the compounds of this disclosure can include all stereochemical isomers (and racemic mixtures) arising from the substitution of these compounds.

### II. Ink (or Digital Toughening Agent)

In some cases, the ink is amenable to inkjet printing. While inkjet printing can enable high degrees of thickness and spatial control during application, the requirements for inkjet compositions can also be extensive. High resolution inkjet printing typically requires curable compositions to have low viscosities, rapid curing rates, and high storage stabilities. As a further requirement for digital printing, inkjet printable compositions often must comprise sufficient adhesive capacities to bind to discretely printed segments and layers. In many cases, the ink not only meets these requirements, but provides favorable properties (e.g., high modulus and elongation at break). Furthermore, in many cases, the ink is photocurable, such that a method of applying the ink can comprise inkjet application followed by irradiation to fix the compositions in desired distributions (e.g., as thin layers, linear segments (e.g., straight or curved lines with defined lengths, widths, and thicknesses), or dots along a surface of a printed substrate).

The ink of the present disclosure can comprise a low viscosity to enable spray and droplet-based application, and prevent inkjet printhead and nozzle clogging. In some cases, the ink comprises a viscosity of at most about 500 mPa*s, at most about 300 mPa*s, at most about 200 mPa*s, at most about 150 mPa*s, at most about 100 mPa*s, at most about 80 mPa*s, at most about 60 mPa*s, at most about 40 mPa*s, at most about 25 mPa*s, at most about 20 mPa*s, at most about 15 mPa*s, at most about 12 mPa*s, at most about 10 mPa*s, at most about 8 mPa*s, at most about 6 mPa*s, or at most about 5 mPa*s at 25° C. In some cases, the ink comprises a viscosity from about 5 mPa*s to about 50 mPa*s. In some cases, the ink comprises a viscosity from about 5 mPa*s to about 30 mPa*s. In some cases, the ink comprises a viscosity from about 10 mPa*s to about 80 mPa*s. In some cases, the ink comprises a viscosity from about 20 mPa*s to about 100 mPa*s. In some cases, the ink comprises a viscosity from about 30 mPa*s to about 150 mPa*s. In some cases, the ink comprises a viscosity from about 3 mPa*s and 50 mPa*s.

Owing to the low viscosities and thin application thicknesses of many inkjet-applied layers disclosed herein, rapid curing rates can be essential for fixing these layers in desired distributions. The ink of the present disclosure can have a t₉₅%, denoting the time required for 95% polymerization, of at most about 90 seconds, at most about 60 seconds, at most about 45 seconds, at most about 30 seconds, at most about 20 seconds, at most about 15 seconds, at most about 12 seconds, at most about 10 seconds, at most about 8 seconds, at most about 6 seconds, at most about 5 seconds, at most about 4 seconds, or at most about 3 seconds when disposed in 30 micron layers and exposed to 14 mW/cm² irradiation at a wavelength sufficient for photopolymerization. The ink can have a polymerization rate of at least about 5%/s, at least about 8%/s, at least about 10%/s, at least about 15%/s, at least about 20%/s, at least about 25%/s, at least about 30%/s, at least about 35%/s, or at least about 40%/s when disposed in 30 micron layers and exposed to 14 mW/cm² irradiation at a wavelength sufficient for photopolymerization.

The ink can utilize a range of polymerizable species. In some cases, the ink utilizes chain growth polymerization, such as anionic polymerization, cationic polymerization, radical polymerization, or ring opening polymerization. In some cases, the ink utilizes step growth polymerization, for example forming polyesters, polyamides, polyurethanes, polyureas, polysiloxanes, polycarbonates, polysulfides, polytriazoles, polyethers, or polythioethers during polymerization.

While a range of polymerizable compositions can be used as the ink as disclosed herein, a surprising discovery of the present disclosure is that thiol-ene-based curable compositions are particularly well suited for these applications. Accordingly, in some embodiments disclosed herein, the ink is a curable thiol-ene composition. As used herein, a curable thiol-ene composition can utilize thiol-olefin coupling for polymerization. For many of the thiol-ene-based curable compositions disclosed herein, thiol-olefin coupling is the predominant polymerization mechanism, such that during curing, at least about 50%, at least about 60%, at least about 70%, at least about 80%, at least about 90%, at least about 95%, at least about 98%, or at least about 99% of the bonds formed during curing result from thiol-olefin coupling. The thiol-olefin coupling can comprise radical addition, Michael addition, or a combination of mechanisms thereof.

Differing from some olefin-containing compositions, which can have relatively high reactivities which lead to diminished polymerization and undesired side product generation, an advantage of thiol-ene-based curable composition can be oxygen insensitivity. To guarantee high purity thiol-ene step-growth polymerization and prevent oxygen inhibition, a thiol-ene based composition of the present disclosure can utilize ene-monomers with low homopolymerization tendencies, such as norbornenes, allyl ethers, and vinyl ethers, whose electron-rich olefins inhibit radical-type homopolymerization and enable copolymerization with thiols. The low oxygen reactivities can contribute to high conversion rates by the inks, such that the inks can exhibit double bond conversion (DBC) rates of at least 80%, at least 85%, at least 90%, at least 93%, at least 94%, at least 95%, or at least 96%.

A thiol-ene based curable composition can comprise a combination of polythiol and polyolefin monomers. As thiol-ene chemistry stoichiometrically converts thiols and olefins to thioether bonds in a 1:1:1 ratio, chain-propagating monomers typically comprise at least two functional groups (e.g., at least two olefins or at least two thiols), while monomers with three or more functional groups affect crosslinking.

Accordingly, in many cases, the ink of the present disclosure comprises a first difunctional monomer comprising two ethylenically unsaturated functional groups and a second difunctional monomer comprising two thiol functional groups. In many cases, the ink is photocurable. In many cases, the molar quantity of thiol groups is comparable to the number of ethylenically unsaturated groups in the ink. In many cases, a molar ratio of ethylenically unsaturated groups and thiol groups in the ink is between about 2:5 and about 5:2. In some cases, the molar ratio of ethylenically unsaturated groups and thiol groups in the ink is between about 2:1 and 1:2. In some cases, the molar ratio of ethylenically unsaturated groups and thiol groups in the ink is between about 3:2 and 2:3. In some cases, the molar ratio of ethylenically unsaturated groups and thiol groups in the ink is between about 4:3 and 3:4. In some cases, the molar ratio of ethylenically unsaturated groups and thiol groups in the ink is between 21:20 and 20:21. In some cases, at least 60%, at least 70%, at least 80%, at least 90%, at least 95%, at least 98%, or at least 99% of polymerization of the ink during curing is thiol-ene polymerization. In some cases, at least 90% of polymerization of the ink during curing is thiol-ene polymerization.

In some cases, the ink further comprises a crosslinker comprising at least three thiol or at least three ethylenically unsaturated functional groups. In some cases, the crosslinker comprises at least four thiol or at least four ethylenically unsaturated functional groups. In some cases, the crosslinker comprises at least five thiol or at least five ethylenically unsaturated functional groups. In some cases, the crosslinker comprises three thiol groups. In some cases, the crosslinker comprises at least three thiol groups. In some cases, the crosslinker comprises at least four thiol groups. In some cases, the crosslinker comprises at least five thiol groups. In some cases, the ink comprises between about 1 and about 75 wt % of the crosslinker. In some cases, the ink comprises between about 1 and about 20 wt %, between about 1 and about 10 wt %, between about 2 and about 30 wt %, between about 2 and about 15 wt %, between about 5 and about 40 wt %, between about 5 and about 25 wt %, between about 5 and about 15 wt %, between about 10 and about 50 wt %, between about 10 and about 30 wt %, between about 10 and about 20 wt %, between about 15 and about 35 wt %, between about 15 and about 25 wt %, between about 20 and about 40 wt %, or between about 20 and about 30 wt % of the crosslinker. In some cases, between about 1% and about 50% of the polymerizable groups in the ink are present in the crosslinker. In some cases, between about 1% and about 20%, between about 1% and about 10%, between about 2% and about 30%, between about 2% and about 15%, between about 5% and about 50%, between about 5% and about 30%, between about 5% and about 20%, between about 10% and about 50%, between about 10% and about 30%, between about 10% and about 20%, between about 15% and about 35%, between about 15% and about 25%, between about 20% and about 40%, or between about 20% and about 30% of the polymerizable groups of the ink are present in the crosslinker.

In some cases, the ethylenically unsaturated groups comprise vinyl ether, an allyl ether, a strained or bridged cyclic alkene, an acrylate, a methacrylate, an acrylamide, a methacrylamide, a trans-butadiene, a 1-butene, a butyl acrylate, a sec-butyl acrylate, a benzyl acrylate, a butyl methacrylate, a butyl vinyl ether, a cis-chlorobutadiene, a trans-chlorobutadiene, a 2-cyanoethyl acrylate, a cyclohexyl acrylate, a diethylaminoethyl methacrylate, an isobutyl acrylate, an isobutylene, an isobutyl vinyl ether, a cis-isoprene, a trans-isoprene, an isotatic isopropyl acrylate, a 2-methoxyethyl acrylate, a methyl acrylate, a methyl vinyl ether, a octadecyl methacrylate, a 1-octene, an octyl methacrylate, a dodecyl acrylate, a dodecyl methacrylate, a dodecyl vinyl ether, a 2-ethoxyethyl acrylate, an ethyl acrylate, a 2-ethylhexyl acrylate, a 2-ethylhexyl methacrylate, a 2-ethylhexyl vinyl ether, an ethyl vinyl ether, a hexyl acrylate, a hexadecyl methacrylate, a hexyl methacrylate, an atactic propylene, an isotactic propylene, a sydiotatic propylene, a propyl vinyl ether, a 2,2,2-trifluoroethyl acrylate, a vinylidene chloride, a vinylidene fluoride, a vinyl propionate, a derivative thereof, or a combination thereof. In some cases, the ethylenically unsaturated groups comprise a vinyl ether, an allyl ether, a strained or bridged cyclic alkene, a derivative thereof, or a combination thereof. In some cases, the ethylenically unsaturated groups comprise a vinyl ether. In particular cases, the first difunctional monomer comprises a divinyl ether.

In some cases, the ethylenically unsaturated groups of the first difunctional monomer, the crosslinker, or both the first difunctional monomer and the crosslinker are separated by at least 5 atoms, by at least 6 atoms, by at least 8 atoms, by at least 10 atoms, by at least 12 atoms, by at least 14 atoms, by at least 18 atoms, by at least 22 atoms, or by at least 25 atoms, as defined by the fewest number of bonds between the closest polymerizable ethylenically unsaturated groups. In some cases the thiols of the second difunctional monomer, the crosslinker, or both the second difunctional monomer and the crosslinker are separated by at least 5 atoms, by at least 6 atoms, by at least 8 atoms, by at least 10 atoms, by at least 12 atoms, by at least 14 atoms, by at least 18 atoms, by at least 22 atoms, or by at least 25 atoms as defined by the fewest number of bonds between the closest polymerizable thiol groups.

In some cases, the first difunctional monomer, the second difunctional monomer, the crosslinker, or a combination thereof comprise a glycol backbone. The glycol backbone may comprise methylene glycol, ethylene glycol, 1,2-propylene glycol, 1,3-propylene glycol, 1,2-butylene glycol, 1,3-butylene glycol, 1,4-butylene glycol, 2,3-butylene glycol, or a combination thereof. In some cases, the glycol backbone comprises ethylene glycol, 1,2-propylene glycol, 1,3-propylene glycol, 1,2-butylene glycol, 1,3-butylene glycol, a derivative thereof, or a combination thereof. In some cases, the glycol backbone comprises ethylene glycol, 1,2-propylene glycol, 1,3-propylene glycol, a derivative thereof, or a combination thereof. In some cases, the glycol backbone comprises ethylene glycol or a derivative thereof. In some cases, the glycol derivative comprises a C₁-C₆ alkyl, a C₁-C₆ alkenyl, a C₁-C₆ alkynyl, a C₁-C₆ alkoxyl, an aryl, a heteroaryl, a hydroxyl, an amine, a cyano, a thiol, a halogen, or a combination thereof. In some cases, the glycol derivative comprises a C₁-C₆ alkyl, a C₁-C₆ alkoxyl, an aryl, a heteroaryl, a hydroxyl, an amine, a halogen, or a combination thereof. In some cases, the glycol derivative comprises a C₁-C₆ alkyl, a hydroxyl, an amine, a halogen, or a combination thereof. In some cases, the glycol backbone comprises at least one glycol unit, at least two glycol units, at least three glycol units, at least 4 glycol units, at least 5 glycol units, or at least 6 glycol units. In some cases, the glycol backbone comprises between 1 and 8, between 1 and 5, between 1 and 3, between 2 and 7, between 2 and 5, between 2 and 4, between 3 and 7, between 3 and 5, or between 3 and 4 glycol units. In some instances of the first difunctional monomer, all glycol units are disposed between the two ethylenically unsaturated groups. In some instances of the second difunctional monomer, all glycol units are disposed between the two thiol groups.

In certain cases, the first difunctional monomer comprises a divinyl ether with 1-6 glycol backbone units. In certain cases, the divinyl ether comprises 2-4 glycol backbone units. In certain cases, the divinyl ether comprises 3-4 glycol backbone units. In some cases, the first difunctional monomer is triethylene glycol divinyl ether.

In certain cases, the second difunctional monomer comprises 1-6 glycol backbone units. In certain cases, the second difunctional monomer comprises 1-4 glycol backbone units. In certain cases, the second difunctional monomer comprises 1-3 glycol backbone units. In certain cases, the second difunctional monomer comprises 2-3 glycol backbone units. In some cases, the second difunctional monomer is 2,2′-(ethylenedioxy)diethanoethiol.

In some cases, the first difunctional monomer comprises a molecular weight of at least 100 Da, at least 125 Da, at least 150 Da, at least 175 Da, at least 200 Da, at least 225 Da, at least 250 Da, or at least 300 Da. In some cases, the second difunctional monomer comprises a molecular weight of at least 75 Da, at least 100 Da, at least 125 Da, at least 150 Da, at least 175 Da, at least 200 Da, at least 225 Da, at least 250 Da, or at least 275 Da.

In some cases, the crosslinker comprises a molecular weight of at least 250 Da, at least 300 Da, at least 350 Da, or at least 400 Da. In some cases, the crosslinker comprises Trimethylolpropane tris(3-mercaptopropionate).

In some cases, the ink comprises a polymer with a molecular weight of at least 1 kDa, at least 2 kDa, at least 5 kDa, at least 10 kDa, at least 20 kDa, at least 30 kDa, at least 50 kDa, at least 75 kDa, or at least 100 kDa. In some cases, the polymer comprises a saccharide. In some cases, the polymer comprises cellulose acetate butyrate (CAB). In some cases, the ink does not contain added solid material, such as nanoparticles, fibers, or powders. In some cases, the ink comprises a further species disclosed herein, such as a photoinitiator or a stabilizer.

### III. Matrix

The present disclosure provides a range of compositions amenable for use as the matrix. Such compositions can be printable with a range of methods, including photolithography, extrusion, sheet lamination, injection molding, and combinations thereof. The matrix can be photo-curable, thermo-curable, chemically curable, or a combination thereof. As described herein, the matrix can include one or more species of polymerizable compounds of the present disclosure (e.g., 1, 2, 3, or more different species), one or more species of polymerizable monomers (e.g., reactive diluents), one or more photoinitiators, one or more toughness modifiers, one or more viscosity modifiers, one or more glass transition temperature modifiers, as well as combinations thereof.

The matrix can have a plurality of components, which may be homogeneously or heterogeneously dispersed therethrough. In many cases, a solid material is homogeneously dispersed throughout a curable composition. As curing can fix the solid material in place within a resulting material, surface exposed or extruding solid material can be removed from uncured, partially cured, or cured composition. The curable composition can comprise a single phase or a plurality of phases. The solid material may be evenly or unevenly distributed across multiple phases. In some cases, the solid material is heterogeneously distributed in a first phase and homogeneously distributed in a second phase of the curable composition. In some cases, the solid material comprises different heterogeneous distributions in separated phases of the curable composition. The curable composition can comprise an emulsion. In some such cases, the solid material can be co-localized with or distributed irrespective of emulsion phases.

In some cases, the matrix comprises: 20 to 60 wt %, based on the total weight of the matrix, of a glass transition temperature modifier; 20 to 50 wt %, based on the total weight of the matrix, of a toughness modifier wherein the toughness modifier is a polymerizable oligomer having a number average molecular weight of greater than 10 kDa; 5 to 80 wt %, based on the total weight of the matrix, of a reactive diluent, wherein the reactive diluent is a polymerizable compound having a molecular weight of 0.1 to 0.5 kDa; and 0.1 to 40 wt %, based on the total weight of the matrix, of a photoinitiator; wherein the viscosity of the matrix is 1 to 50 mPa*s at 75° C.

### 1) Reactive Diluents

The matrix can comprise a polymerizable monomer homogenously or heterogenously dispersed or patterned therethrough. The degree of heterogenous partitioning (e.g., emulsification) or homogeneity can be controlled with a method or device disclosed herein, for example through agitation prior to resin sheet printing. Such polymerizable monomers can be used as reactive diluents. In many cases, the polymerizable monomer comprises an ethylenically unsaturated functional group capable of undergoing polymerization. In some cases, the ethylenically unsaturated group comprises a vinyl ether, a vinyl amine, a styryl, an acryloyl, a methacryloyl, an acrylamide, a methacrylamide, or a combination thereof. In some cases, the ethylenically unsaturated group comprises an acryloyl or a methacryloyl group. In some cases, the polymerizable monomer comprises an aromatic group.

In various cases, a polymerizable monomer can comprise an acrylate or methacrylate moiety for incorporation into an oligomeric or polymeric backbone, coupled to a linear or cyclic (e.g., mono-, bi-, or tricyclic) side-chain moiety. Generally, any aliphatic, cycloaliphatic or aromatic molecule with a mono-functional polymerizable reactive functional group can be used (also includes liquid crystalline monomers). In some instances, the polymerizable reactive functional groups is an acrylate or methacrylate group. In some embodiments, however, no or only low amounts (e.g., 5% w/w or less) of a reactive diluent may be used.

A reactive diluent of the matrix typically has a low viscosity. One or more reactive diluents may be included in the matrix to reduce viscosity, e.g., to a viscosity less than the viscosity of the matrix in the absence of the reactive diluent. The reactive diluent(s) may reduce the viscosity of the composition by at least 10%, such as by at least 20%, at least 30%, at least 40%, at least 50%, at least 60%, at least 70%, at least 80%, or at least 90%. The matrix can comprise 5 to 80 wt %, 5 to 70 wt %, 5 to 60 wt %, 5 to 50 wt %, 5 to 40 wt %, 5 to 30 wt %, 5 to 25 wt %, 5 to 20 wt %, 10 to 70 wt %, 10 to 60 wt %, 10 to 50 wt %, 10 to 40 wt %, 10 to 30 wt %, 10 to 25 wt %, 20 to 70 wt %, 20 to 60 wt %, 20 to 50 wt %, 20 to 40 wt %, 20 to 35 wt %, or 20 to 30 wt %, based on the total weight of the matrix, of the reactive diluent. In certain embodiments, the matrix may comprise 5 to 80 wt %, based on the total weight of the matrix, of the reactive diluent. In certain embodiments, the matrix may comprise 5 to 50 wt %, based on the total weight of the matrix, of the reactive diluent. The reactive diluent of the matrix may be monofunctional. In some embodiments, the reactive diluent comprises a methacrylate. In some embodiments, the reactive diluent comprises an acrylate, a methacrylate, a diacrylate, a dimethacrylate, or a compound comprising an acrylate and a methacrylate. In some embodiments, the reactive diluent comprises a dimethacrylate. The reactive diluent may be selected from the group consisting of dimethacrylates of polyglycols, hydroxybenzoic acid ester (meth)acrylates, and mixtures thereof. Optionally, the reactive diluent is a cycloalkyl 2-, 3- or 4-((meth)acryloxy)benzoate.

In some embodiments, the reactive diluent is a compound of formula (VII):

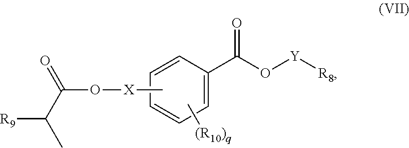

wherein:
R₈ represents optionally substituted C₃-C₁₀ cycloalkyl, optionally substituted 3- to 10-membered heterocycloalkyl, or optionally substituted C₆-C₁₀ aryl;
R₉ represents H or C₁-C₆ alkyl;
each R₁₀ independently represents halo, C₁-C₃ alkyl, C₁-C₃ alkoxy, Si(R₁₁)₃, P(O)(OR₁₂)₂, or N(R₁₃)₂;
each R₁₁ independently represents C₁-C₆ alkyl or C₁-C₆ alkoxy;
each R₁₂ independently represents C₁-C₆ alkyl or C₆-C₁₀ aryl;
each R₁₃ independently represents H or C₁-C₆ alkyl;
X is absent, C₁-C₃ alkylene, 1- to 3-membered heteroalkylene, or (CH₂CH₂O)ᵣ;
Y is absent or C₁-C₆ alkylene;
q is an integer from 0 to 4; and
r is an integer from 1 to 4.

In some embodiments, for a compound of formula (VII), R₈ may be unsubstituted or substituted with one or more substituents selected from the group consisting of C₁-C₆ alkyl, C₁-C₆ alkoxy, C₃-C₇ cycloalkyl, C₆-C₁₀aryl, C₁-C₆-alkoxy-C₆-C₁₀-aryl, —O(CO)-(C₁-C₆)alkyl, —COO—(C₁-C₆)alkyl, ═O, —F, —Cl, and —Br. Specific reactive diluents suitable for use in the matrix are described herein below, including compounds of formula (VII). In some embodiments, the reactive diluent is selected from TEGDMA (triethylene glycol dimethacrylate) (Aldrich), D4MA (1,12-dodecanediol dimethacrylate) (Aldrich), HSMA (3,3,5-trimethylcyclohexyl 2-(methacryloxy)benzoate), BSMA (benzyl salicylate methacrylate), decanediol-1,10-dimethacrylate (D3MA), bisphenol A-glycidyl methacrylate (BisGMA) and urethane dimethacrylate (UDMA). However, small, monofunctional monomers such as 2-Hydroxyethyl methacrylate (HEMA; used for our matrix), 2-Hydroxybutyl methacrylate (HBMA), isobornyl methacrylate (IBMA), 2-Ethylhexyl methacrylate (EHMA), 2-Cyanoethyl methacrylate (CEMA), Benzyl methacrylate (BMA), 2-Phenoxyethyl methacrylate (PEMA), Butyl methacrylate (BMA), Lauryl methacrylate (LMA), Syringyl methacrylate (SMA), and the corresponding acrylates, and a compound of formula (VII).

In some embodiments, for a compound of formula (VII), R₈ is selected from optionally substituted C₅-C₁₀ cycloalkyl and optionally substituted C₆-C₁₀ aryl, such as optionally substituted phenyl. In some embodiments, R₈ is optionally substituted C₅-C₇ cycloalkyl. The optionally substituted C₅-C₇ cycloalkyl may have 5 to 15 carbon atoms in total, such as 5 to 12 or 5 to 10 carbon atoms. For a compound of formula (VII), R₈ may be a monocyclic cycloalkyl, such as cyclohexyl. In some embodiments, R₈ is a bicyclic cycloalkyl, such as a bridged, fused, or spirocyclic cycloalkyl. This includes, for example, bicyclo[2.2.1]heptyl, bicyclo[1.1.1]pentyl, spiro[4.4]nonyl, and decahydronaphthyl, each of which may be optionally substituted. In some embodiments, R₈ is unsubstituted. In some embodiments, R₈ is substituted with at least one substituent.

Exemplary optional substituents of R₈ include C₁-C₆ alkyl, C₁-C₆ alkoxy, C₃-C₇ cycloalkyl, C₆-C₁₀ aryl, C₁-C₆-alkoxy-C₆-C₁₀-aryl, —O(CO)—(C₁-C₆)alkyl, —COO—(C₁-C₆)alkyl, ═O, —F, —Cl, and —Br. In some embodiments, R₈ is substituted with at least one —CH₃. For example, in some embodiments R₈ is substituted with one or more —CH₃ and optionally further substituted with one or more substituents selected from the group consisting of C₁-C₆ alkyl, C₁-C₆ alkoxy, —O(CO)—(C₁-C₆)alkyl, —COO—(C₁-C₆)alkyl, ═O, —F, —Cl, and —Br. In some embodiments, R₈ is substituted with one or more, linear or branched C₁-C₆ alkyl, such as methyl, ethyl, n-propyl, iso-propyl, n-butyl, sec-butyl, iso-butyl, or tert-butyl. Two sub stituents of R₈, such as two C₁-C₆ alkyl, may be connected to form a ring. For example, two substituents on a cyclohexyl group may form a bridge, such as the methylene bridge found in bicyclo[2.2.1]heptyl. In some embodiments, R₈ is substituted with one or more substituents selected from the group consisting of C₁-C₄ alkyl and C₁-C₄ alkoxy.

Exemplary R₈ groups include, but are not limited to

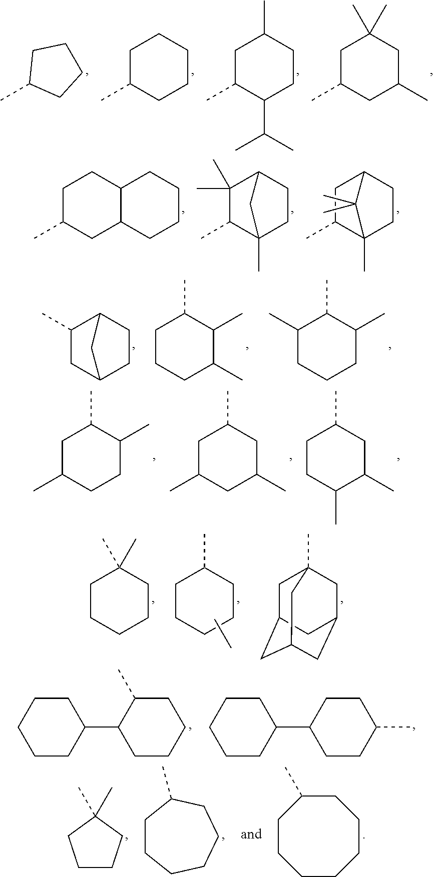

The broken line is used herein to indicate the bond to the rest of the molecule (e.g., the bond to linker Y of formula (VII)). Further exemplary —Y—R₈ groups include, but are not limited to

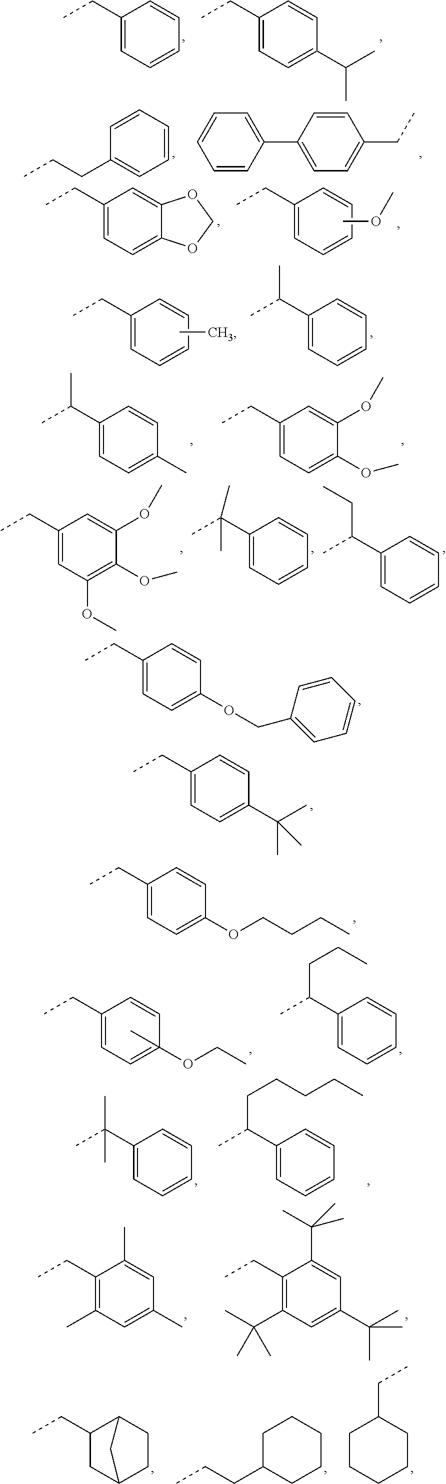

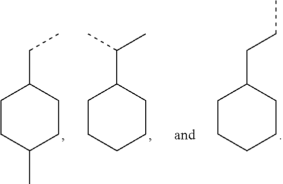

In some embodiments, q is 0 or 1, such as q is 0. In some embodiments, R₉ is H or CH₃. In some embodiments, X is C₁-C₃ alkylene, such as methylene. In some embodiments, X is absent. In some embodiments, Y is C₁-C₃ alkylene.

### 2) Toughness Modifiers

Using a toughness modifier and a reactive diluent may be used to form the matrix can result in the composition being well processible at the processing temperatures usually employed in high temperature lithography-based photopolymerization processes, i.e. temperatures between 90° C. and 120° C., as their viscosities at these temperatures are sufficiently low, despite the presence of the high molecular weight toughness modifier. Moreover, as such curable compositions typically comprise multiple divalent polymerizable components, they result in crosslinked polymers, more specifically in crosslinked polymers having excellent thermomechanical properties, as detailed below.

The toughness modifier and the reactive diluent are typically miscible and compatible in the methods described herein. When used in the subject compositions, the toughness modifier may provide for high elongation at break and toughness via strengthening effects, and the reactive diluent may improve the processability of the formulations, particularly of those comprising high amounts of toughness modifiers, while maintaining high values for strength and T_{g}.

A toughness modifier of the subject compositions may have a low glass transition temperature (T_{g}), such as a T_{g} less than 0° C. In some examples, the T_{g} of the toughness modifier may be less than 25° C., such as less than 15° C., less than 10° C., less than 5° C., less than 0° C., less than −5° C., or less than −10° C. The T_{g} of a polymer or composition described herein may be assessed using dynamic mechanical analysis (DMA) and is provided herein as the tan 6 peak.

The toughness modifier can be a component having a low glass transition temperature (e.g., below 0° C.), which can add to tough behavior if used above its glass transition temperature. The toughness modifier can have a molecular weight greater than 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, 21 kDa, 22 kDa, 23 kDa, 24 kDa, or greater than 25 kDa. In certain embodiments, the toughness modifier can have a molecular weight greater than 5 kDa, such as a molecular weight greater than 10 kDa. The matrix can comprise 10 to 70 wt %, 10 to 60 wt %, 10 to 50 wt %, 10 to 40 wt %, 10 to 30 wt %, 10 to 25 wt %, 20 to 60 wt %, 20 to 50 wt %, 20 to 40 wt %, 20 to 35 wt %, 20 to 30 wt %, 25 to 60 wt %, 25 to 50 wt %, 25 to 45 wt %, 25 to 40 wt %, or 25 to 35 wt %, based on the total weight of the matrix, of the toughness modifier. In certain embodiments, the matrix may comprise 25 to 35 wt %, based on the total weight of the matrix, of the toughness modifier. In certain embodiments, the matrix may comprise 20 to 40 wt %, based on the total weight of the matrix, of the toughness modifier.

The toughness modifier may comprise a polyolefin, a polyester, a polyurethane, a polyvinyl, a polyamide, a polyether, a polyacrylic, a polycarbonate, a polysulfone, a polyarylate, a cellulose-based resin, a polyvinyl chloride resin, a polyvinylidene fluoride, a polyvinylidene chloride, a cycloolefin-based resin, a polybutadiene, a glycidyl methacrylate, or a methyl acrylic ester. For example, the toughness modifier may comprise a urethane group, a carbonate group, or both a urethane group and a carbonate group.

In some embodiments, the toughness modifier comprises at least one methacrylate group, such as at least two methacrylate groups. In some embodiments, the toughness modifier comprises at least one acrylate. The toughness modifier can be an acrylate selected from an epoxy acrylate (e.g., a Bisphenol A epoxy acrylate), an epoxy methacrylate (e.g., a Bisphenol A epoxy methacrylate), a novolac type epoxy acrylate (e.g., cresol novolac epoxy acrylate or phenol novolac epoxy acrylate), a modified epoxy acrylate (e.g., phenyl epoxy acrylate, aliphatic alkyl epoxy acrylate, soybean oil epoxy acrylate, Photocryl® DP296, Photocryl® E207/25TP, Photocryl® E207/25HD, or Photocryl® E207/30PE), a bisphenol A-glycidyl methacrylate (bis-GMA), a urethane acrylate, an aliphatic urethane acrylate (e.g., aliphatic difunctional acrylate, aliphatic trifunctional acrylate, an acrylate vinyl ester, an aliphatic multifunctional acrylate), an aromatic urethane acrylate (e.g., aromatic difunctional acrylate, aromatic trifunctional acrylate, an aromatic multifunctional acrylate), an urethane dimethacrylate, a polyester acrylate (e.g., trifunctional polyester acrylate, tetrafunctional polyester acrylate, difunctional polyester acrylate, hexafunctional polyester acrylate), a silicone acrylate (e.g., silicone urethane acrylate, silicone polyester acrylate), a melamine acrylate, a dendritic acrylate, an acrylic acrylate, a caprolactone monomer acrylate (e.g., caprolactone methacrylate, caprolactone acrylate), a dodecandediol dimethacrylate, an oligo amine acrylate (e.g., amine acrylate, aminated polyester acrylate), a derivative thereof, or a combination thereof. Non-limiting examples of aliphatic urethane acrylates include difunctional aliphatic acrylates (e.g., Miramer PU210, Miramer PU2100, Miramer PU2560, Miramer SC2404, Miramer SC2565, Miramer UA5216, Miramer U307, Miramer U3195, or Photocryl DP102), trifunctional aliphatic acyrlates (e.g., Miramer PU320, Miramer PU340, Miramer PU3450, Miramer U375, or Photocryl DP225), tetrafunctional aliphatic acrylates (e.g., Miramer U3304), hexafunctional aliphatic acrylates (e.g., Miramer MU9800), and multifunctional aliphatic acrylates (e.g., Miramer MU9800 or Miramer SC2152). In some cases, the toughness modifier comprises an acrylate vinyl ester. In some cases, the toughness modifier comprises urethane dimethacrylate, bisphenol A-glycidyl methacrylate (bis-GMA), dodecandediol dimethacrylate, or a combination thereof.

In some embodiments, the toughness modifier comprises acrylic monomers selected from n-butyl acrylate, iso-decyl acrylate, n-decyl methacrylate, n-dodecyl acrylate, n-dodecyl methacrylate, 2-ethylhexyl acrylate, 2-(2-ethoxyethoxy)ethyl acrylate, n-hexyl acrylate, 2-methoxyethylacrylate, n-octyl methacrylate, 2-phenylethyl acrylate, n-propyl acrylate, and tetrahydrofurfuryl acrylate. In some embodiments, the toughness modifier is a poly(ethersulfone), a poly(sulfone), a poly(etherimide), or a combination thereof. In certain embodiments, the toughness modifier is a polypropylene or a polypropylene derivative. In some embodiments, the toughness modifier is a rubber or a rubber derivative. In certain embodiments, the toughness modifier is a polyethylene or a derivative thereof. In some embodiments, the toughness modifier comprises fluorinated acrylic monomers, which can be selected from 1H,1H-heptafluorobutyl acrylate, 1H,1H,3H-hexafluorobutyl acrylate, 1H,1H,5H-octafluoropentyl acrylate, or 2,2,2-trifluoroethyl acrylate.

In some embodiments, the toughness modifier is acetaldehyde, allyl glycidyl ether, trans-butadiene, 1-butene, butyl acrylate, sec-butyl acrylate, benzyl acrylate, butyl glydicyl ether, butyl methacrylate, butyl vinyl ether, ϵ-caprolactone, cis-chlorobutadiene, trans-chlorobutadiene, 2-cyanoethyl acrylate, cyclohexyl acrylate, diethylaminoethyl methacrylate, isobutyl acrylate, isobutylene, isobutyl vinyl ether, cis-isoprene, trans-isoprene, isotatic isopropyl acrylate, 2-methoxyethyl acrylate, methyl acrylate, methyl glicidyl ether, methylphenylsiloxane, methyl vinyl ether, octadecyl methacrylate, 1-octene, octyl methacrylate, dimethylsiloxane, dodecyl acrylate, dodecyl methacrylate, dodecyl vinyl ether, epibromohydrin, epichlorohydrin, 1,2-epoxybutane, 1,2-epoxydecane, 1,2-epoxyoctane, 2-ethoxyethyl acrylate, ethyl acrylate, HDPE ethylene, ethylene adipate, ethylene-trans-1,4-cyclohexyldicarboxylate, ethylene malonate, ethylene oxide, 2-ethylhexyl acrylate, 2-ethylhexyl methacrylate, 2-ethylhexyl vinyl ether, ethyl vinyl ether, formaldehyde, hexyl acrylate, hexadecyl methacrylate, hexyl methacrylate, atactic propylene, isotactic propylene, sydiotatic propylene, propylene oxide, propyl vinyl ether, tetrahydrofuran, tetramethylene adipate, 2,2,2-trifluoroethyl acrylate, trimethylene oxide, vinylidene chloride, vinylidene fluoride, vinyl propionate, a derivative thereof, or a combination thereof.

In some embodiments, the toughness modifier (also referred to herein as the toughening modifier) comprises a chlorinated polyethylene, a methacrylate, a copolymer of a chlorinated polyethylene and methacrylate, a derivative thereof, or a combination thereof. In some embodiments, the toughening modifier is a rubber powder. In some embodiments, the toughening modifier is an anhydride-grafted polymer, an anhydride polymer, or a combination thereof containing epoxy groups. In certain embodiments, the anhydride-grafted polymer is a grafted anhydride-modified thermoplastic elastomer, and can comprise a styrene-based thermoplastic elastomer comprising styrene units and units of an olefin (e.g. ethylene, propylene or butene), such as a styrene-butadiene-styrene (SBS), styrene-isoprene-styrene (SIS), styrene-ethylene-butadiene-styrene (SEBS), styrene-ethylene-propylene-styrene (SEPS) copolymers. Suitable anhydrides include unsaturated carboxylic acid anhydride, wherein the carboxylic acid is an acrylic acid, methacrylic acid, α-methyl acrylic acid, maleic acid, fumaric acid, itaconic acid, citraconic acid, tetrahydrophthalic acid, methyl-tetrahydrophthalic acid, or combinations thereof.

In some cases, the toughness modifier comprises a structure according to formula (II), (III), (IV), or (V), wherein:

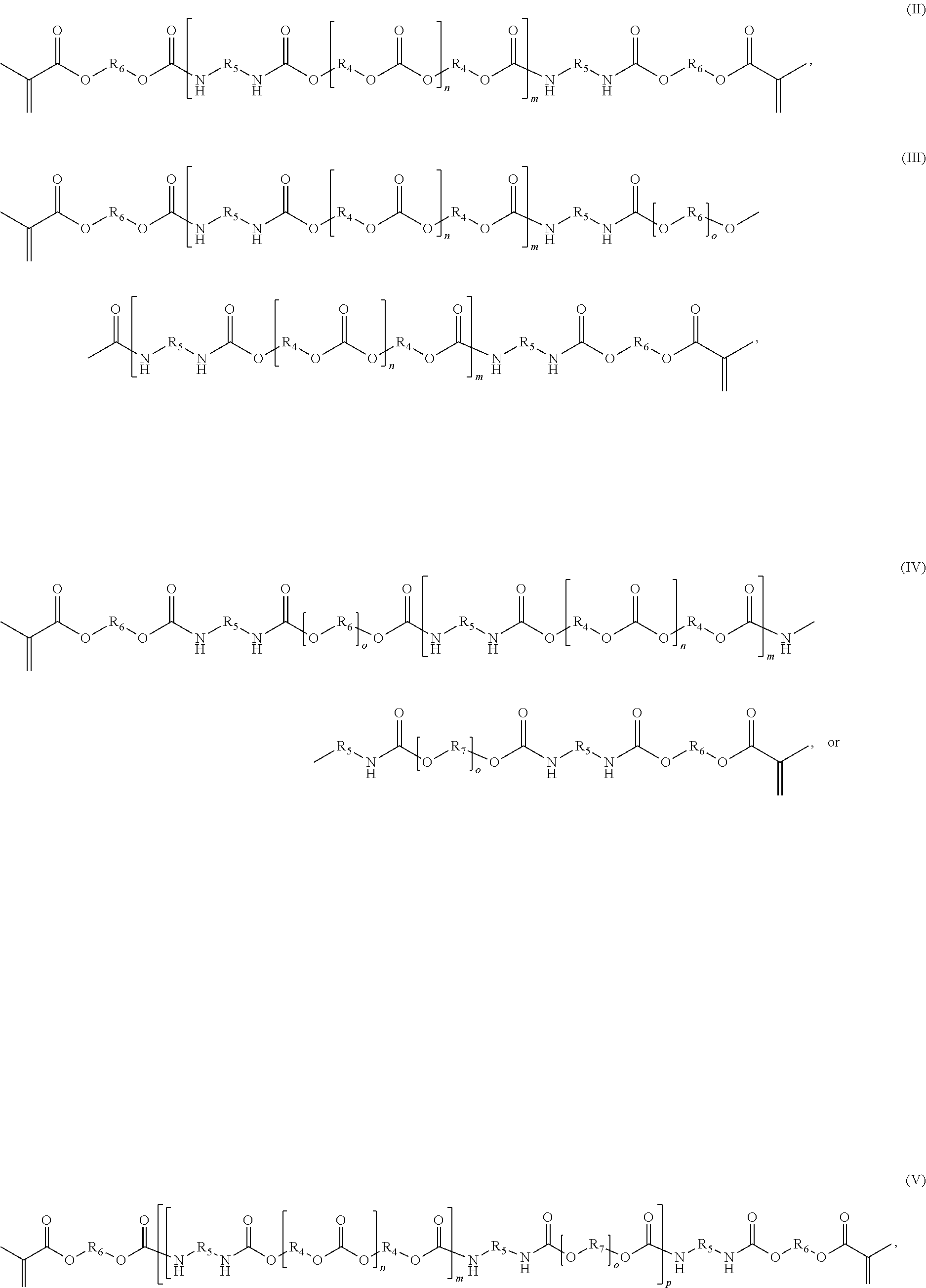

wherein:
each R₄ and each R₅ independently represent a divalent, linear, branched or cyclic C₅-C₁₅ aliphatic radical,
each R₆ independently represents a divalent, linear or branched C₂-C₄ alkyl radical,
each R₇ independently represents a divalent, linear or branched C₂-C₆ alkyl radical,
each n is independently an integer from 1 to 10,
each m is independently an integer from 1 to 20,
each o is independently an integer from 5 to 50, and
p is an integer from 1 to 40,
with the proviso that R₄, R₅, R₆, R₇, n, m, o and p are selected so as to result in a number average molecular weight of the (poly)carbonate-(poly)urethane dimethacrylate greater than 5 kD.

Specific toughness modifiers suitable for use in the matrix are described herein below, including compounds of formula (II), (III), (IV) or (V). In some embodiments, the toughness modifier is selected from UA5216 (Miwon), a compound of formula (II), a compound of formula (III), a compound of formula (IV), a compound of formula (V), TNM1, TNM2, TNM3, TNM4, TNMS, and TNM6.

In certain aspects, R₅ is a divalent radical originating from a diisocyanate selected from the group consisting of isophorone diisocyanate (IPDI), hexamethylene diisocyanate (HDI), trimethylhexamethylene diisocyanate (2,2,4- and 2,4,4-mixture, TMDI), dicyclohexylmethane 4,4′-diisocyanate (HMDI), 1,3-bis(isocyanatomethyl)cyclohexane, and mixtures thereof.

In some aspects, R₆ is a divalent radical originating from a diol independently selected from the group consisting of 1,2-ethanediol, 1,3-propanediol, 1,2-propanediol, 1,4-butanediol, and mixtures thereof. In certain aspects, R₆ is a divalent radical originating from 1,2-ethanediol.

In certain aspects, R₄ is a divalent radical originating from a diol selected from the group consisting of 2,2-dimethyl-1,3-propanediol (DMP), 1,6-hexanediol, 1,4-cyclohexanedimethanol (CHDM), and mixtures thereof. In some aspects, R₄ is the alcoholic moiety of a polycarbonate.

### 3) Crosslinkers

In some embodiments, the matrix of the present disclosure comprises a crosslinker. The crosslinker may have a sufficient number of polymerizable groups to affect crosslinking. The crosslinker may comprise at least two, at least three, at least four, at least five, or at least six polymerizable groups. The matrix may comprise from about 0.5 to about 60 wt %, from about 0.5 to 20 wt %, from about 1 to about 30 wt %, from about 1 to about 10 wt %, from about 1 to about 5 wt %, from about 5 to about 50 wt %, from about 5 to about 20 wt %, from about 10 to about 60 wt %, from about 10 to about 30 wt %, from about 25 to about 50 wt %, from about 30 to about 50 wt %, or from about 40 to about 60 wt % of the crosslinker.

### 4) Glass Transition Temperature Modifiers

The matrix of the present disclosure may further comprise 0 to 50 wt %, based on the total weight of the composition, of a glass transition temperature (T_{g}) modifier (also referred to herein as a T_{g} modifier, a glass transition modifier, a crosslinker, and a cross-linker). The T_{g} modifier can have a high glass transition temperature, which leads to a high heat deflection temperature, which can be necessary to use a material at elevated temperatures. In some embodiments, the matrix comprises 0 to 80 wt %, 0 to 75 wt %, 0 to 70 wt %, 0 to 65 wt %, 0 to 60 wt %, 0 to 55 wt %, 0 to 50 wt %, 1 to 50 wt %, 2 to 50 wt %, 3 to 50 wt %, 4 to 50 wt %, 5 to 50 wt %, 10 to 50 wt %, 15 to 50 wt %, 20 to 50 wt %, 25 to 50 wt %, 30 to 50 wt %, 35 to 50 wt %, 0 to 40 wt %, 1 to 40 wt %, 2 to 40 wt %, 3 to 40 wt %, 4 to 40 wt %, 5 to 40 wt %, 10 to 40 wt %, 15 to 40 wt %, or 20 to 40 wt % of a T_{g} modifier. In certain embodiments, the matrix comprises 0-50 wt % of a T_{g} modifier. The T_{g} modifier typically has a higher T_{g} than the toughness modifier. Optionally, the number average molecular weight of the T_{g} modifier is 0.4 to 5 kDa. In some embodiments, the number average molecular weight of the T_{g} modifier is from 0.1 to 5 kDa, from 0.2 to 5 kDa, from 0.3 to 5 kDa, from 0.4 to 5 kDa, from 0.5 to 5 kDa, from 0.6 to 5 kDa, from 0.7 to 5 kDa, from 0.8 to 5 kDa, from 0.9 to 5 kDa, from 1.0 to 5 kDa, from 0.1 to 4 kDa, from 0.2 to 4 kDa, from 0.3 to 4 kDa, from 0.4 to 4 kDa, from 0.5 to 4 kDa, from 0.6 to 4 kDa, from 0.7 to 4 kDa, from 0.8 to 4 kDa, from 0.9 to 4 kDa, from 1 to 4 kDa, from 0.1 to 3 kDa, from 0.2 to 3 kDa, from 0.3 to 3 kDa, from 0.4 to 3 kDa, from 0.5 to 3 kDa, from 0.6 to 3 kDa, from 0.7 to 3 kDa, from 0.8 to 3 kDa, from 0.9 to 3 kDa, or from 1 to 3 kDa. The toughness modifier, the reactive diluent and the T_{g} modifier are typically miscible and compatible in the methods described herein. When used in the matrix, the T_{g} modifier may provide for high T_{g} and strength values, sometimes at the expense of elongation at break. The toughness modifier may provide for high elongation at break and toughness via strengthening effects, and the reactive diluent may improve the processability of the formulations, particularly of those comprising high amounts of toughness modifiers, while maintaining high values for strength and T_{g}.

The T_{g} modifier may comprise a urethane group. In some embodiments, the T_{g} modifier comprises at least one methacrylate group. The matrix may comprise 10 to 20 wt %, based on the total weight of the matrix, of the T_{g} modifier. The T_{g} modifier may comprise a urethane group. In some embodiments, the T_{g} modifier comprises at least one methacrylate group. The matrix may comprise 20 to 40 wt %, based on the total weight of the matrix, of the T_{g} modifier. The T_{g} modifier may comprise a urethane group. In some embodiments, the T_{g} modifier comprises at least one methacrylate group. The matrix may comprise 10 to 50 wt %, based on the total weight of the matrix, of the T_{g} modifier. Specific T_{g} modifiers suitable for use in the matrix are described herein below, including compounds of formula (I). In some embodiments, the T_{g} modifier is selected from H1188 (bis((2-((methacryloyloxy)methyl)octahydro-1H-4,7-methanoinden-5-yl)methyl) cyclohexane-1,4-dicarboxylate), TGM1, TGM2, TGM3, TGM4, and a compound of formula (I). In some embodiments, the T_{g} modifier is a derivative of H1188 (DMI), TGM1, TGM2, TGM3, TGM4, or a derivative of the compound of formula (I). In some embodiments, the T_{g} modifier is a blend of modifiers comprising H1188 (DMI), TGM1, TGM2, TGM3, TGM4, or a compound of formula (I). In some embodiments, the T_{g} modifier is H1188:

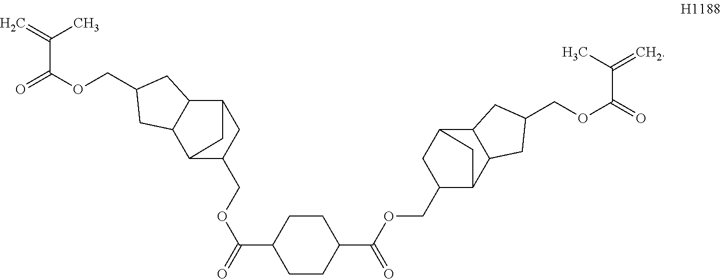 [SMILES: C=C(C)C(=O)OCC1CC2C3CC(COC(=O)C4CCC(C(=O)OCC5CC6CC5C5CC(COC(=O)C(=C)C)CC65)CC4)C(C3)C2C1]

In some embodiments, the T_{g} modifier is TGM1:

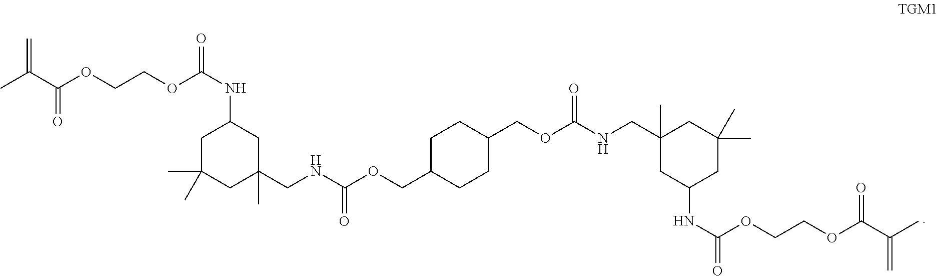 [SMILES: C=C(C)C(=O)OCCOC(=O)NC1CC(C)(C)CC(C)(CNC(=O)OCC2CCC(COC(=O)NCC3(C)CC(NC(=O)OCCOC(=O)C(=C)C)CC(C)(C)C3)CC2)C1]

In some embodiments, the T_{g} modifier is TGM2:

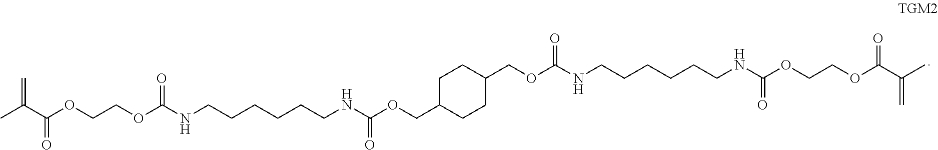 [SMILES: C=C(C)C(=O)OCCOC(=O)NCCCCCCNC(=O)OCC1CCC(COC(=O)NCCCCCCNC(=O)OCCOC(=O)C(=C)C)CC1]

In some embodiments, the T_{g} modifier is TGM3:

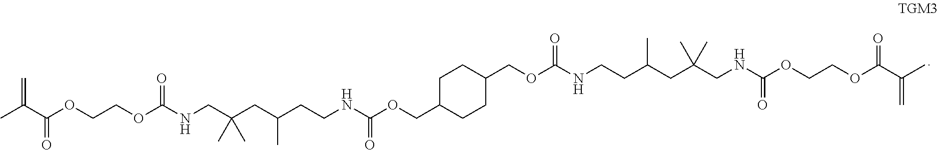 [SMILES: C=C(C)C(=O)OCCOC(=O)NCC(C)(C)CC(C)CCNC(=O)OCC1CCC(COC(=O)NCCC(C)CC(C)(C)CNC(=O)OCCOC(=O)C(=C)C)CC1]

In some embodiments, the T_{g} modifier is TGM4:

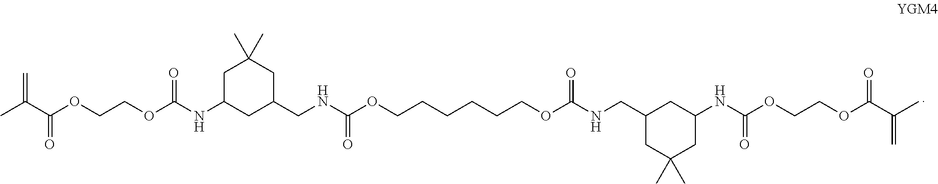 [SMILES: C=C(C)C(=O)OCCOC(=O)NC1CC(CNC(=O)OCCCCCCOC(=O)NCC2CC(NC(=O)OCCOC(=O)C(=C)C)CC(C)(C)C2)CC(C)(C)C1]

In some embodiments, the T_{g} modifier is a compound of Formula (I):

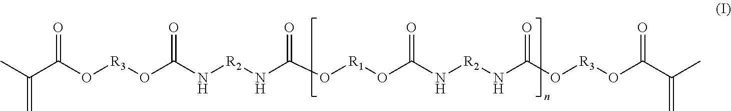

wherein:
each R₁ and each R₂ independently represent a divalent, linear, branched or cyclic C₅ -C₁₅ aliphatic radical, with the proviso that at least one of R₁ and R₂ is or comprises a C₅ -C₆ cycloaliphatic structure,
each R₃ independently represents a divalent, linear or branched C₂-C₄ alkyl radical, and
n is an integer from 1 to 5,
with the proviso that R₁, R₂, R₃ and n are selected so as to result in a number average molecular weight of the oligomeric dimethacrylate from 0.4 to 5 kDa.

In some embodiments, the T_{g} modifier comprises a plurality of aliphatic rings. In certain embodiments, the T_{g} modifier comprises a plurality of aliphatic rings. In some embodiments, the aliphatic rings are hydrocarbon rings. In some embodiments, the aliphatic rings are saturated. In some embodiments, the plurality of aliphatic rings comprise cyclobutane, cyclopentane, cyclohexane, cycloheptane, cyclooctane, cyclononane, cyclodecane, or any combination thereof. In some embodiments, the plurality of aliphatic rings include bridged ring structures. In some embodiments, the plurality of aliphatic rings include fused ring structures. In certain embodiments, the middle portion of the T_{g} modifier comprises a cyclohexane-1,4-dicarboxylic acid, a cyclohexanedimethanol, a cyclohexane-1,4-diylbis(methylene) dicarbamate, or a combination thereof. In certain embodiments, the center of the Tg modifier structure comprises a cyclohexane-1,4-diylbis(methylene) dicarbamate (e.g., TGM1, TGM2, and TGM3).

In some embodiments, the T_{g} modifier comprises a methacrylate. In some embodiments, the T_{g} modifier comprises at least two methacrylates. In certain embodiments, the T_{g} modifier has terminal portions comprising methacrylates. In some embodiments, the T_{g} modifier has a structure that terminates at each end with a methacrylate. In some embodiments, the T_{g} modifier is a bis(2-methacrylate) (e.g., TGM1, TGM2, TGM3, TGM4, and H1188).

In some embodiments, the T_{g} modifier comprises a blend of components, selected from TGM1, TGM2, TGM3, TGM4, H1188, a compound of formula (I), D3MA (1,10-decanediol dimethacrylate), D4MA (1,12-dodecanediol dimethacrylate), RDI, LPU624, a derivative thereof, or a combination thereof.

In some embodiments, the T_{g} modifier comprises Bomar XR-741MS.

### 5) Photoinitiators

In various embodiments, the matrix disclosed herein is a photo-curable matrix. Such photo-curable matrix described herein can further comprise one or more photoinitiators. Such photoinitiator, when activated with light of an appropriate wavelength (e.g., UV/VIS) can initiate a polymerization reaction (e.g., during photo-curing) between the monomers, toughness modifiers, and other potentially polymerizable components that may be present in the photo-curable matrix, to form a polymeric material as further described herein. Generally, photoinitiators described in the present disclosure can include those that can be activated with light and initiate polymerization of the polymerizable components of the formulation. A “photoinitiator”, as used herein, may generally refer to a compound that can produce radical species and/or promote radical reactions upon exposure to radiation (e.g., UV or visible light).

In some embodiments, a photo-curable matrix herein comprises 0.05 to 1 wt %, 0.05 to 2 wt %, 0.05 to 3 wt %, 0.05 to 4 wt %, 0.05 to 5 wt %, 0.1 to 1 wt %, 0.1 to 2 wt %, 0.1 to 3 wt %, 0.1 to 4 wt %, 0.1 to 5 wt %, 0.1 to 6 wt %, 0.1 to 7 wt %, 0.1 to 8 wt %, 0.1 to 9 wt %, or 0.1 to 10 wt %, based on the total weight of the matrix, of a photoinitiator. In some embodiments, the photoinitiator is a free radical photoinitiator. In certain embodiments, the free radical photoinitiator comprises an alpha hydroxy ketone moiety (e.g., 2-hydroxy-2-methylpropiophenone or 1-hydroxycyclohexyl phenyl ketone), an alpha-amino ketone (e.g., 2-benzyl-2-(dimethylamino)-4′-morpholinobutyrophenone or 2-methyl-1-[4-(methylthio)phenyl]-2-morpholinopropan-1-one), 4-methyl benzophenone, an azo compound (e.g., 4,4′-Azobis(4-cyanoyaleric acid), 1,1′-Azobis(cyclohexanecarbonitrile, Azobisisobutyronitrile, 2,2′-Azobis(2-methylpropionitrile), or 2,2′-Azobis(2-methylpropionitrile)), an inorganic peroxide, an organic peroxide, or any combination thereof. In some embodiments, the matrix comprises a photoinitiator comprising acylphosphine oxides including TPO, SpeedCure TPO-L (ethyl(2,4,6-trimethylbenzoyl)phenyl phosphinate) and Irgacure819, and acylgermanes including Ivocerin.

In some embodiments, a photo-curable matrix comprises a photoinitiator selected from a benzophenone, a mixture of benzophenone and a tertiary amine containing a carbonyl group which is directly bonded to at least one aromatic ring, and an Irgacure (e.g., Irgacure 907 (2-methyl-1-[4-(methylthio)-phenyl]-2-morpholino-propanone-1) or Irgacure 651 (2,2-dimethoxy-1,2-diphenylethan-1-one). In some embodiments, the photoinitiator comprises an acetophenone photoinitiator (e.g., 4′-hydroxyacetophenone, 4′-phenoxyacetophenone, 4′-ethoxyaceto-phenone), a benzoin, a benzoin derivative, a benzil, a benzil derivative, a benzophenone (e.g., 4-benzoylbiphenyl, 3,4-(dimethylamino)benzophenone, 2-methylbenzophenone), a cationic photoinitiator (e.g., diphenyliodonium nitrate, (4-iodophenyl)diphenylsulfonium triflate, triphenylsulfonium triflate), an anthraquinone, a quinone (e.g., camphorquinone), a phosphine oxide, a phosphinate, 9,10-phenanthrenequinone, a thioxanthone, any combination thereof, or any derivative thereof.

In some embodiments, the photoinitiator can have a maximum wavelength absorbance between 200 and 300 nm, between 300 and 400 nm, between 400 and 500 nm, between 500 and 600 nm, between 600 and 700 nm, between 700 and 800 nm, between 800 and 900 nm, between 150 and 200 nm, between 200 and 250 nm, between 250 and 300 nm, between 300 and 350 nm, between 350 and 400 nm, between 400 and 450 nm, between 450 and 500 nm, between 500 and 550 nm, between 550 and 600 nm, between 600 and 650 nm, between 650 and 700 nm, or between 700 and 750 nm. In some embodiments, the photoinitiator has a maximum wavelength absorbance between 300 to 500 nm.

### 6) Additives

The matrix of the present disclosure (e.g., the first composition) may further comprise 0.1 to 10 wt %, based on the total weight of the matrix, of an additive. Additives may increase the performance or processibility of the composition in direct or additive manufacturing processes. The additive may be selected from a resin, a defoamer and a surfactant, or a combination thereof. A resin included in the composition as an additive may be highly functional, which may reduce the time to gel. One or more defoamers may be added to the composition to reduce foam in the formulation, which may lead to fewer defects (e.g., air pockets) in a polymer prepared from the composition. A surfactant may be added to reduce surface tension of the composition, which may improve processing in an additive manufacturing process, such as 3D-printing. In some embodiments, the composition comprises from 0.01 to 20 wt %, from 0.01 to 15 wt %, from 0.01 to 10 wt %, from 0.01 to 9 wt %, from 0.01 to 8 wt %, from 0.01 to 7 wt %, from 0.01 to 6 wt %, from 0.01 to 5 wt %, from 0.1 to 10 wt %, from 0.1 to 9 wt %, from 0.1 to 8 wt %, from 0.1 to 7 wt %, from 0.1 to 6 wt %, from 0.1 to 5 wt %, from 0.5 to 10 wt %, from 0.5 to 9 wt %, from 0.5 to 8 wt %, from 0.5 to 7 wt %, from 0.5 to 6 wt %, from 0.5 to 5 wt %, from 1 to 10 wt %, from 1 to 9 wt %, from 1 to 8 wt %, from 1 to 7 wt %, from 1 to 6 wt %, or from 1 to 5 wt %, based on the total weight of the composition, of an additive. In some embodiments, the composition comprises 0.3 to 3.5 wt %, based on the total weight of the composition, of an additive. In some embodiments, the defoamer comprises a modified urea (e.g., BYK®-7411 ES, BYK®-7420 ES, and BYK®-7410 ET), a silicone-free foam-destroying polymer (e.g., BYK®-A 535), a composition having a short siloxane backbone and long organic modifications (e.g., TEGO® RAD 2100), a silica-base defoamer, a hydrophobic silica, a wax, a fatty alcohol, a fatty acid, or a wetting component (e.g., a silicone-free wetting compound, such as TEGO® Wet 510). In some embodiments, the defoamer is selected from the group consisting of BYK®-7411 ES, BYK®-7420 ES, BYK®-7410 ET, BYK®-A 535, TEGO® RAD2100, and TEGO® WET510. In some embodiments, the additive is a surfactant selected from the group consisting of an amphoteric surfactant, a zwitterionic surfactant, an anionic surfactant, a nonionic surfactant, a cationic surfactant, or any combination thereof. The cationic surfactant is selected from quaternary salts, certain amines and combinations thereof. In some embodiments, the additive is selected from SIU2400 (Miwon), BDT1006 (Dymax), BYK®-430, and BYK®-A535.

In some embodiments, the composition further comprises 0.05 to 1 wt %, 0.05 to 2 wt %, 0.05 to 3 wt %, 0.05 to 4 wt %, 0.05 to 5 wt %, 0.1 to 1 wt %, 0.1 to 2 wt %, 0.1 to 3 wt %, 0.1 to 4 wt %, 0.1 to 5 wt %, 0.1 to 6 wt %, 0.1 to 7 wt %, 0.1 to 8 wt %, 0.1 to 9 wt %, or 0.1 to 10 wt %, based on the total weight of the composition, of a photoblocker. The photoblocker can absorb irradiation and prevent or decrease the rate of polymerization or degradation, and its addition to the curable composition can increase the resolution of printable materials. In certain embodiments, the photoblocker comprises a hydroquinone, 1,4-dihydroxybenzene, a compound belonging to the HALS (hindered-amine light stabilizer) family, a benzophenone, a benzotriazole, any derivative thereof, or any combination thereof. In some embodiments, the photoblocker comprises 2,2′-dihydroxy-4-methoxybenzophenone. In certain embodiments, the photoblocker is selected from the group consisting of Michler's ketone, 4-Allyloxy-2-hydroxybenzophenone 99%, 2-(2H-Benzotriazol-2-yl)-4,6-bis(1-methyl-1-phenylethyl)phenol powder, 2-(2H-Benzotriazol-2-yl)-4,6-di-tert-pentylphenol, 2-(2H-Benzotriazol-2-yl)-6-dodecyl-4-methylphenol, 2-[3-(2H-Benzotriazol-2-yl)-4-hydroxyphenyl]ethyl methacrylate, 2-(2H-Benzotriazol-2-yl)-4-methyl-6-(2-propenyl)phenol, 2-(2H-Benzotriazol-2-yl)-4-(1,1,3,3-tetramethylbutyl)phenol, 2-(4-Benzoyl-3-hydroxyphenoxy)ethyl acrylate, 3,9-Bis(2,4-dicumylphenoxy)-2,4,8,10-tetraoxa-3,9-diphosphaspiro[5.5]undecane, Bis(octadecyl)hydroxylamine powder, 3,9-Bis(octadecyloxy)-2,4,8,10-tetraoxa-3,9-diphosphaspiro[5.5]undecane, Bis(1-octyloxy-2,2,6,6-tetramethyl-4-piperidyl) sebacate, Bis(2,2,6,6-tetramethyl-4-piperidyl) sebacate, 2-tert-Butyl-6-(5-chloro-2H-benzotriazol-2-yl)-4-methylphenol, 2-tert-Butyl-4-ethylphenol, 5-Chloro-2-hydroxybenzophenone, 5-Chloro-2-hydroxy-4-methylbenzophenone, 2,4-Di-tert-butyl-6-(5-chloro-2H-benzotriazol-2-yl)phenol, 2,6-Di-tert-butyl-4-(dimethylaminomethyl)phenol, 3′,5′-Dichloro-2′-hydroxyacetophenone, Didodecyl 3,3′-thiodipropionate, 2,4-Dihydroxybenzophenone, 2,2′-Dihydroxy-4-methoxybenzophenone, 2′,4′-Dihydroxy-3′-propylacetophenone, 2,3-Dimethylhydroquinone, 2-(4,6-Diphenyl-1,3,5-triazin-2-yl)-5-Rhexyl)oxyl-phenol, 5-Ethyl-1-aza-3,7-dioxabicyclo[3.3.0]octane, Ethyl 2-cyano-3,3-diphenylacrylate, 2-Ethylhexyl 2-cyano-3,3-diphenylacrylate, 2-Ethylhexyl trans-4-methoxycinnamate, 2-Ethylhexyl salicylate, 2-Hydroxy-4-(octyloxy)benzophenone, Menthyl anthranilate, 2-Methoxyhydroquinone, Methyl-p-benzoquinone, 2,2′-Methylenebis[6-(2H-benzotriazol-2-yl)-4-(1,1,3,3-tetramethylbutyl)phenol], 2,2′-Methylenebis(6-tert-butyl-4-ethylphenol), 2,2′-Methylenebis(6-tert-butyl-4-methylphenol), 5,5′-Methylenebis(2-hydroxy-4-methoxybenzophenone), Methylhydroquinone, 4-Nitrophenol sodium salt hydrate, Octadecyl 3-(3,5-di-tert-butyl-4-hydroxyphenyl)propionate, Pentaerythritol tetrakis(3,5-di-tert-butyl-4-hydroxyhydrocinnamate), 2-Phenyl-5-benzimidazolesulfonic acid, Poly[[6-[(1,1,3,3-tetramethylbutyl)amino]-s-triazine-2,4-diyl]-[(2,2,6,6-tetramethyl-4-piperidyl)imino]-hexamethylene-[(2,2,6,6-tetramethyl-4-piperidyl)imino], Sodium D-isoascorbate monohydrate, Tetrachloro-1,4-benzoquinone, Triisodecyl phosphite, 1,3,5-Trimethyl-2,4,6-tris(3,5-di-tert-butyl-4-hydroxybenzyl)benzene, Tris(4-tert-butyl-3-hydroxy-2,6-dimethylbenzyl) isocyanurate, Tris(2,4-di-tert-butylphenyl) phosphite, 1,3,5-Tris(2-hydroxyethyl)isocyanurate,and Tris(nonylphenyl) phosphite.

In some embodiments, the photoblocker has a maximum wavelength absorbance between 200 and 300 nm, between 300 and 400 nm, between 400 and 500 nm, between 500 and 600 nm, between 600 and 700 nm, between 700 and 800 nm, between 800 and 900 nm, between 150 and 200 nm, between 200 and 250 nm, between 250 and 300 nm, between 300 and 350 nm, between 350 and 400 nm, between 400 and 450 nm, between 450 and 500 nm, between 500 and 550 nm, between 550 and 600 nm, between 600 and 650 nm, between 650 and 700 nm, or between 700 and 750 nm. In some embodiments, the photoblocker has a maximum wavelength absorbance between 300 to 500 nm, such as 300 to 400 nm or 350 to 480 nm.

In some cases, the additive is a stabilizer. The stabilizer may enhance a lifespan of a composition by preventing polymerization reactions prior to initiation, by preventing side reactions prior to or during curing, or a combination thereof. In some cases, the stabilizer is a radical scavenger. In some cases, the stabilizer is an oxygen scavenger. In some cases, the stabilizer comprises pyrogallol.

In some embodiments, the additive is a branched dendritic oligomer. In some embodiments, the additive has one or more, two or more, three or more, four or more, five or more, six or more, seven or more, eight or more, nine or more, ten or more, or greater than 10 functional groups. In some embodiments, the additive has one or more, two or more, three or more, four or more, five or more, six or more, seven or more, eight or more, nine or more, ten or more, or greater than 10 acrylate functional groups. In certain embodiments, the branched dendritic oligomer additive is a dendritic acrylate oligomer. In some embodiments, the dendritic acrylate oligomer is Bomar™ BDT-1006, Bomar™ BDT-1018, Bomar™ BDT-4330, and the like. In some embodiments, the multi-functional additive comprises a silicone urethane acrylate. As a non-limiting example, the silicone urethane acrylate can have 1 functional group, 2 functional groups, 3 functional groups, 4 functional groups, 5 functional groups, 6 functional groups, 7 functional groups, 8 functional groups, 9 functional groups, 10 functional groups, 11 functional groups, 12 functional groups, 13 functional groups, 14 functional groups, 15 functional groups, 16 functional groups, 17 functional groups, 18 functional groups, 19 functional groups, 20 functional groups, or greater than 20 functional groups. In some embodiments, the additive can be a silicone urethane acrylate or comprises a silicone urethane acrylate. As a non-limiting example, the silicone urethane acrylate can have 1 acrylate group, 2 acrylate groups, 3 acrylate groups, 4 acrylate groups, 5 acrylate groups, 6 acrylate groups, 7 acrylate groups, 8 acrylate groups, 9 acrylate groups, 10 acrylate groups, 11 acrylate groups, 12 acrylate groups, 13 acrylate groups, 14 acrylate groups, 15 acrylate groups, 16 acrylate groups, 17 acrylate groups, 18 acrylate groups, 19 acrylate groups, 20 acrylate groups, or greater than 20 acrylate groups. As non-limiting examples of silicone acrylates, the additive can be Miramer SIU2400 (a silicone urethane acrylate having a functionality number of 10, diluted with 10% TPGDA) or SIP910 (a silicone polyester acrylate having a functionality number of 2).

### IV. Printing Methods

Digital materials of the present disclosure can be formed using a variety of printing methods. In some aspects, successive layers of material are deposited and “cured in place”. In many instances, the printing comprises providing successive layers or regions comprising different compositions, printing methods, or both. In many instances, the successive layers are provided in a repeating pattern. For example, two or more compositions (e.g., the matrix and inks) may be printed in repeating, alternating layers (e.g., A-B-A-B-A-B . . . , A-B-B-A-B-B-A-B-B . . . , A-B-C-A-B-C-A-C . . . , etc.). The digital materials can also comprise compositions arranged in discrete regions, such as pixels within a 3-dimensional grid. The digital materials can comprise empty space, for example openings within a weave of polymeric linear segments.

A variety of printing techniques may be used to generate such materials, including photolithography, stereolithography, selective laser sintering (SLS), fused deposition modeling (FDM), jetting, extrusion, build platform-based printing techniques, and combinations thereof. In many embodiments, selective laser sintering involves using a laser beam to selectively melt and fuse a layer of powdered material according to a desired cross-sectional shape in order to build up the object geometry. In many embodiments, fused deposition modeling involves melting and selectively depositing a thin filament of thermoplastic polymer in a layer-by-layer manner in order to form an object. In yet another example, 3D printing can be used to fabricate the appliances herein. In many embodiments, 3D printing involves jetting or extruding one or more materials onto a build surface in order to form successive layers of the object geometry.

The printing can also comprise a continuous direct fabrication method, wherein a direct fabrication process achieves continuous build-up of an object by continuous movement of the build platform (e.g., along the vertical or Z-direction) during an irradiation phase, such that the hardening depth of the irradiated photopolymer is controlled by build platform movement. In yet another example, a continuous direct fabrication method utilizes a “heliolithography” approach in which the liquid photopolymer is cured with focused radiation while the build platform is continuously rotated and raised. Accordingly, the object geometry can be continuously built up along a spiral build path. A further example of continuous direct fabrication method can involve extruding a composite material composed of a curable liquid material surrounding a solid strand, wherein the composite material can be extruded along a continuous three-dimensional path in order to form the object.

Photopolymerization, as disclosed herein, can comprise exposure of light of sufficient power and of a wavelength capable of initiating polymerization. The wavelengths and power of light useful to initiate polymerization depends on the initiator used. Light as used herein includes any wavelength and power capable of initiating polymerization. Preferred wavelengths of light include ultraviolet (UV) or visible. UV light sources include UVA (wavelength about 400 nm to about 320 nm), UVB (about 320 nm to about 290 nm) or UVC (about 290 nm to about 100 nm). Any suitable source may be used, including laser sources. The source may be broadband or narrowband, or a combination thereof. The light source may provide continuous or pulsed light during the process. Both the length of time the system is exposed to UV light and the intensity of the UV light can be varied to determine the ideal reaction conditions. Photopolymers may be fabricated by “vat” processes in which light is used to selectively cure a vat or reservoir of the photopolymer. Each layer of photopolymer may be selectively exposed to light in a single exposure or by scanning a beam of light across the layer. Specific techniques include stereolithography (SLA), Digital Light Processing (DLP) and two photon-induced photopolymerization (TPIP).

In specific embodiments disclosed herein, the printing comprises lithography for the first composition and inkjet printing for the second composition. Such a tandem technique can simultaneously benefit from high resolution of the lithography and thin-layer deposition capabilities of the inkjet printing.

### 1) Methods for Printing Digital Materials

Aspects of the present disclosure provide a method for printing a digital material comprising: curing a first portion of a matrix to form a first layer of an object; applying an ink to the first layer of the object; and curing the ink, thereby forming a second layer disposed on the first layer of the object. In many cases, the ink comprises a storage stability of at least 20 days at 25° C. In some cases, the storage stability comprises an increase in viscosity of less than 50%. In some cases, the storage stability comprises retention of at least 75% of a maximum possible conversion rate of the ink during polymerization. In some cases, the storage is in the absence of light.

In many instances, the curing the ink comprises thiol-ene step-growth polymerization. In many cases, the curing the first portion of the matrix, the curing the ink, or both comprises photo-curing. In some cases, both the curing of the matrix and the curing of the ink comprise photo-curing, but utilize different wavelengths of light. In many instances, the second layer is adherent to the first layer. In many cases, the second layer comprises a dot, a linear segment, a continuous layer, or a combination thereof. In some cases, the second layer is fully contiguous with the first layer. In other cases, the second layer is partially contiguous with the first layer. In some cases, the method further comprises repeating the method to form a plurality of the first and the second layers of the object.

In some cases, the first layer and the second layer each comprise a thickness of between about 5 and about 250 microns (m). In some cases, the first layer comprises a thickness of between about 100 and 2000 microns. In some cases, the first layer comprises a thickness of between about 200 and about 1200 microns. In some cases, the second layer comprises a thickness of between about 5 and about 500 microns. In some cases, the second layer comprises a thickness of between about 25 and about 250 microns. In some cases, the second layer comprises a thickness of between about 10 and about 150 microns. In some cases, the second layer comprises a thickness of between about 10 and about 50 microns. In some cases, the second layer comprises a thickness of between about 5 and about 30 microns.

In many instances, the curing the ink is performed in the presence of molecular oxygen. An advantage of many of the curable compositions disclosed herein, including certain thiol-ene compositions, is inertness towards molecular oxygen during curing. While many curable compositions offer high theoretical polymerization rates (i.e., the percentage of polymerizable groups converted during curing), oxygen reactivity can lead to quenching and side product formation which can result in substantially lower degrees of polymerization. Many of the curable compositions herein overcome this limitation by exhibiting substantial oxygen unreactivity prior to (leading to high storage stabilities) and during curing. In some cases, the matrix or the ink is unreactive towards molecular oxygen (0 2) during curing, such that the polymerization rate achieved in the presence of molecular oxygen (e.g., atmospheric levels of O₂) are within at least 10% of the polymerization rate achieved in the absence of molecular oxygen.

In some cases (for example, in many instances in which the curing the first portion of the matrix comprises photolithography), the method further comprises separating the first layer of the object from a second portion of the matrix not cured during the curing. In such cases, the separating can comprise removing the object or the portion thereof from a vat comprising the second portion of the matrix.

In some cases, the ink cures rapidly. As many of the curable compositions disclosed herein comprise low viscosities (e.g., in some cases less than the about 40 mPa*s viscosity of olive oil), rapid curing can be essential for controlling spatial resolution and thickness. In some cases, the curing the ink reaches at least 95% completion after at most 60 seconds. In some cases, the curing the ink reaches at least 95% completion after at most 45 seconds. In some cases, the curing the ink reaches at least 95% completion after at most 30 seconds. In some cases, the curing the ink reaches at least 95% completion after at most 20 seconds. In some cases, the curing the ink reaches at least 95% completion after at most 10 seconds. In some cases, the curing the ink reaches at least 90% completion after at most 10 seconds. In some cases, the curing the ink reaches at least 80% completion after at most 10 seconds.

In many cases, the applying the ink to the first layer of the object comprises inkjet printing. As the inkjet printing can enable high degrees of control over the spatial distribution and thickness of the second curable material, the ink can comprise a thickness of less than 300 microns, less than 250 microns, less than 200 microns, less than 150 microns, less than 100 microns, less than 75 microns, less than 50 microns, less than 40 microns, less than 30 microns, less than 25 microns, less than 20 microns, less than 15 microns, or less than 10 microns. Furthermore, as inkjet printing often requires relatively low viscosity compositions, the ink can comprise a viscosity of less than about 150 mPa*s, less than about 120 mPa*s, less than about 100 mPa*s, less than about 75 mPa*s, less than about 50 mPa*s, less than about 40 mPa*s, less than about 30 mPa*s, less than about 25 mPa*s, less than about 20 mPa*s, less than about 15 mPa*s, or less than about 10 mPa*s.

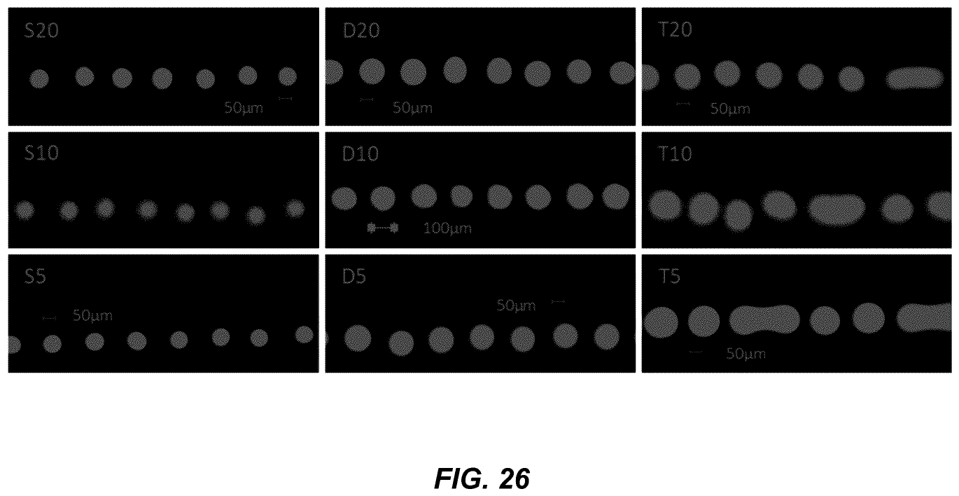

In some cases, the inkjet printing of the ink comprises a resolution of at most about 500 microns, at most about 300 microns, at most about 200 microns, at most about 150 microns, at most about 100 microns, at most about 75 microns, or at most about 50 microns. As printing resolution is often limited by stability, viscosity, and curing rate, the compositions of the present disclosure can enable high degrees of printing resolution (e.g., as shown in FIG. 26) not readily achievable with many commercially available curable compositions.

In some cases, the ink comprises a high storage stability. Long shelf-life can be important for spray and droplet-based printing techniques, such as inkjet printing, as minor increases in viscosity can clog printheads and diminish printing resolution or prevent printing entirely. In some cases, the ink increases in viscosity by no more than 20% when stored at 25° C. for 20 days in the absence of light. In some cases, the ink increases in viscosity by no more than 30% when stored at 25° C. for 20 days in the absence of light. In some cases, the ink increases in viscosity by no more than 40% when stored at 25° C. for 20 days in the absence of light. In some cases, the ink increases in viscosity by no more than 50% when stored at 25° C. for 20 days in the absence of light. In some cases, the ink increases in viscosity by no more than 60% when stored at 25° C. for 20 days in the absence of light. In some cases, the ink increases in viscosity by no more than 80% when stored at 25° C. for 20 days in the absence of light. In some cases, the ink increases in viscosity by no more than 100% when stored at 25° C. for 20 days in the absence of light.

As some forms of printing will heat the ink prior to printing, in some cases, the ink is stable during storage at elevated temperatures. In some cases, the ink increases in viscosity by no more than 20%, no more than 40%, no more than 60%, no more than 80%, or no more than 100% when stored at 37° for 1 day in the absence of light. In some cases, the ink increases in viscosity by no more than 20%, no more than 40%, no more than 60%, no more than 80%, or no more than 100% when stored at 37° for 3 days in the absence of light. In some cases, the ink increases in viscosity by no more than 20%, no more than 40%, no more than 60%, no more than 80%, or no more than 100% when stored at 37° for 7 days in the absence of light. In some cases, the ink increases in viscosity by no more than 20%, no more than 40%, no more than 60%, no more than 80%, or no more than 100% when stored at 50° for 1 day in the absence of light. In some cases, the ink increases in viscosity by no more than 20%, no more than 40%, no more than 60%, no more than 80%, or no more than 100% when stored at 50° for 3 days in the absence of light. In some cases, the ink increases in viscosity by no more than 20%, no more than 40%, no more than 60%, no more than 80%, or no more than 100% when stored at 50° for 7 days in the absence of light.

In some cases, as disclosed further herein, the ink comprises a vinyl ether and a thiol. In some cases, the vinyl ether is a divinyl ether and the thiol is a dithiol. In some cases, the vinyl ether comprises tri(ethylene glycol) divinyl ether (TEGDVE). In some cases, the thiol comprises 2,2′-(ethylenedioxy diethanethiol (EDDT). In some cases, the ink further comprises: (a) a crosslinking agent, (b) a photoinitiator, (c) a stabilizer, (d) a filler, (e) a dye, or (f) any combination of (a)-(e) thereof. In some cases, the crosslinking agent comprises 1,1,1-tris-(hydroxymethyl)-propane-tris-(3-mercaptopropionate) TMPMP. In some cases, a ratio of thiols and olefins in the ink is between about 5:2 or 2:5, between about 2:1 and about 1:2, between about 3:2 and about 2:3, between about 4:3 and about 3:4, between about 5:4 and about 4:5, or between about 21:20 and about 20:21. In some cases, the ink cures to at least 95% completion after at most 10 seconds. In some cases, the ink is inert towards molecular oxygen (O₂) during curing. In some cases, the ink further comprises a crosslinking agent, a photoinitiator, a stabilizer, a filler, a dye, or any combination thereof.

In some cases, upon curing, the ink comprises a final storage modulus of between about 120 MPa and 360 MPa; a final loss modulus of between about 40 MPa and about 70 MPa; a maximal shrinkage force of between about 2 N and about 4.5 N; a maximal elongation break of between about 14% and 34%; a mass increase of at most about 32% upon exposure to aqueous or organic solvent; an elastic-modulus of between about 3 MPa and about 6 MPa; a hardness of at between about 0.75 MPa and 2 MPa; or a combination thereof. In many cases, the curing the first composition comprises photolithography, stereolithography, extrusion, sheet lamination, injection molding, thermoforming, or a combination thereof. In many instances, the curing the first portion of the matrix comprises photolithography. In some cases, the lithography comprises stereolithography. In some cases, the lithography comprises moving a build platform. In some cases, the matrix comprises an acrylate or a methacrylate monomer. In some cases, upon curing, the matrix comprises: (A) a flexural modulus greater than or equal to 50 MPa, 100 MPa, or 200 MPa; (B) an elastic modulus of greater than or equal to 150 MPa, 250 MPa, 350 MPa, 450 MPa, 550 MPa, or between about 500 and 1000 MPa, or from about 550 to about 800 MPa; (C) an elongation at break greater than or equal to 5% before and after 24 hours in a wet environment at 37° C.; (D) a water uptake of less than 25 wt % when measured after 24 hours in a wet environment at 37° C.; (E) transmission of at least 30% of visible light through the polymeric material after 24 hours in a wet environment at 37° C.; (F) a plurality of polymeric phases, wherein at least one polymeric phase of the one or more polymeric phases has a T_{g} of at least 60° C., 80° C., 90° C., 100° C., or at least 110° C.; or (G) any combination of (A)-(F). In some cases, the matrix further comprises a crosslinking agent, a photoinitiator, a stabilizer, a filler, a dye, or any combination thereof.

### 2) General Methods for Printing Curable Compositions

The various embodiments of printed devices presented herein can be fabricated in a wide variety of ways. In many cases, portions or layers of a device are generated with a printing method outlined herein, and then coated with an inkjet printed composition. In some embodiments, the orthodontic appliances herein (or portions thereof) can be produced using direct fabrication, such as additive manufacturing techniques (also referred to herein as “3D printing”) or subtractive manufacturing techniques (e.g., milling). In some embodiments, direct fabrication involves forming an object (e.g., an orthodontic appliance or a portion thereof) without using a physical template (e.g., mold, mask etc.) to define the object geometry. Additive manufacturing techniques can be categorized as follows: (1) vat photopolymerization (e.g., stereolithography), in which an object is constructed layer by layer from a vat of liquid photopolymer composition; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer by layer; (5) powder bed fusion, including but not limited to direct metal laser sintering (DMLS), electron beam melting (EBM), selective heat sintering (SHS), selective laser melting (SLM), and selective laser sintering (SLS); (6) sheet lamination, including but not limited to laminated object manufacturing (LOM) and ultrasonic additive manufacturing (UAM); and (7) directed energy deposition, including but not limited to laser engineering net shaping, directed light fabrication, direct metal deposition, and 3D laser cladding. For example, stereolithography can be used to directly fabricate one or more of the appliances herein. In some embodiments, stereolithography involves selective polymerization of a photosensitive composition according to a desired cross-sectional shape using light (e.g., ultraviolet light). The object geometry can be built up in a layer-by-layer fashion by sequentially polymerizing a plurality of object cross-sections. As another example, the appliances herein can be directly fabricated using selective laser sintering. In some embodiments, selective laser sintering involves using a laser beam to selectively melt and fuse a layer of powdered material according to a desired cross-sectional shape in order to build up the object geometry. As yet another example, the appliances herein can be directly fabricated by fused deposition modeling. In some embodiments, fused deposition modeling involves melting and selectively depositing a thin filament of thermoplastic polymer in a layer-by-layer manner in order to form an object. In yet another example, material jetting can be used to directly fabricate the appliances herein. In some embodiments, material jetting involves jetting or extruding one or more materials onto a build surface in order to form successive layers of the object geometry.

Alternatively or in combination, some embodiments of the appliances herein (or portions thereof) can be produced using indirect fabrication techniques, such as by thermoforming over a positive or negative mold. Indirect fabrication of an appliance can involve producing a positive or negative mold of the patient's dentition in a target arrangement (e.g., by rapid prototyping, milling, etc.) and thermoforming one or more sheets of material over the mold in order to generate an appliance shell.

In some embodiments, the direct fabrication methods provided herein build up the object geometry in a layer-by-layer fashion, with successive layers being formed in discrete build steps. Alternatively or in combination, direct fabrication methods that allow for continuous build-up of an object geometry can be used, referred to herein as “continuous direct fabrication.” Various types of continuous direct fabrication methods can be used. As an example, in some embodiments, the appliances herein are fabricated using “continuous liquid interphase printing,” in which an object is continuously built up from a reservoir of photopolymerizable composition by forming a gradient of partially cured composition between the building surface of the object and a polymerization-inhibited “dead zone.” In some embodiments, a semi-permeable membrane is used to control transport of a photopolymerization inhibitor (e.g., oxygen) into the dead zone in order to form the polymerization gradient. Continuous liquid interphase printing can achieve fabrication speeds about 25 times to about 100 times faster than other direct fabrication methods, and speeds about 1000 times faster can be achieved with the incorporation of cooling systems. Continuous liquid interphase printing is described in U.S. Patent Publication Nos. 2015/0097315, 2015/0097316, and 2015/0102532, the disclosures of each of which are incorporated herein by reference in their entirety.

As another example, a continuous direct fabrication method can achieve continuous build-up of an object geometry by continuous movement of the build platform (e.g., along the vertical or Z-direction) during the irradiation phase, such that the hardening depth of the irradiated photopolymer is controlled by the movement speed. Accordingly, continuous polymerization of material on the build surface can be achieved. Such methods are described in U.S. Pat. No. 7,892,474, the disclosure of which is incorporated herein by reference in its entirety.

In another example, a continuous direct fabrication method can involve extruding a composite material composed of a curable liquid material surrounding a solid strand. The composite material can be extruded along a continuous three-dimensional path in order to form the object. Such methods are described in U.S. Patent Publication No. 2014/0061974, the disclosure of which is incorporated herein by reference in its entirety.

In yet another example, a continuous direct fabrication method utilizes a “heliolithography” approach in which the liquid photopolymer is cured with focused radiation while the build platform is continuously rotated and raised. Accordingly, the object geometry can be continuously built up along a spiral build path. Such methods are described in U.S. Patent Publication No. 2014/0265034, the disclosure of which is incorporated herein by reference in its entirety.

The direct fabrication approaches provided herein are compatible with a wide variety of materials, including but not limited to one or more of the following: a polyester, a co-polyester, a polycarbonate, a thermoplastic polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate, a styrenic block copolymer (SBC), a silicone rubber, an elastomeric alloy, a thermoplastic elastomer (TPE), a thermoplastic vulcanizate (TPV) elastomer, a polyurethane elastomer, a block copolymer elastomer, a polyolefin blend elastomer, a thermoplastic co-polyester elastomer, a thermoplastic polyamide elastomer, a thermoset material, or combinations thereof. The materials used for direct fabrication can be provided in an uncured form (e.g., as a liquid, resin, powder, etc.) and can be cured (e.g., by photopolymerization, light curing, gas curing, laser curing, crosslinking, etc.) in order to form an orthodontic appliance or a portion thereof. The properties of the material before curing may differ from the properties of the material after curing. Once cured, the materials herein can exhibit sufficient strength, stiffness, durability, biocompatibility, etc. for use in an orthodontic appliance. The post-curing properties of the materials used can be selected according to the desired properties for the corresponding portions of the appliance.

In some embodiments, relatively rigid portions of the orthodontic appliance can be formed via direct fabrication using one or more of the following materials: a polyester, a co-polyester, a polycarbonate, a thermoplastic polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, and/or a polytrimethylene terephthalate.

In some embodiments, relatively elastic portions of the orthodontic appliance can be formed via direct fabrication using one or more of the following materials: a styrenic block copolymer (SBC), a silicone rubber, an elastomeric alloy, a thermoplastic elastomer (TPE), a thermoplastic vulcanizate (TPV) elastomer, a polyurethane elastomer, a block copolymer elastomer, a polyolefin blend elastomer, a thermoplastic co-polyester elastomer, and/or a thermoplastic polyamide elastomer.

Machine parameters can include curing parameters. For digital light processing (DLP)-based curing systems, curing parameters can include power, curing time, and/or grayscale of the full image. For laser-based curing systems, curing parameters can include power, speed, beam size, beam shape and/or power distribution of the beam. For printing systems, curing parameters can include material drop size, viscosity, and/or curing power. These machine parameters can be monitored and adjusted on a regular basis (e.g., some parameters at every 1-x layers and some parameters after each build) as part of the process control on the fabrication machine. Process control can be achieved by including a sensor on the machine that measures power and other beam parameters every layer or every few seconds and automatically adjusts them with a feedback loop. For DLP machines, gray scale can be measured and calibrated before, during, and/or at the end of each build, and/or at predetermined time intervals (e.g., every nth build, once per hour, once per day, once per week, etc.), depending on the stability of the system. In addition, material properties and/or photo-characteristics can be provided to the fabrication machine, and a machine process control module can use these parameters to adjust machine parameters (e.g., power, time, gray scale, etc.) to compensate for variability in material properties. By implementing process controls for the fabrication machine, reduced variability in appliance accuracy and residual stress can be achieved.

Optionally, the direct fabrication methods described herein allow for fabrication of an appliance including multiple materials, referred to herein as “multi-material direct fabrication.” In some embodiments, a multi-material direct fabrication method involves concurrently forming an object from multiple materials in a single manufacturing step. For instance, a multi-tip extrusion apparatus can be used to selectively dispense multiple types of materials from distinct material supply sources in order to fabricate an object from a plurality of different materials. Such methods are described in U.S. Pat. No. 6,749,414, the disclosure of which is incorporated herein by reference in its entirety. Alternatively or in combination, a multi-material direct fabrication method can involve forming an object from multiple materials in a plurality of sequential manufacturing steps. For instance, a first portion of the object can be formed from a first material in accordance with any of the direct fabrication methods herein, then a second portion of the object can be formed from a second material in accordance with methods herein, and so on, until the entirety of the object has been formed.

Direct fabrication can provide various advantages compared to other manufacturing approaches. For instance, in contrast to indirect fabrication, direct fabrication permits production of an orthodontic appliance without utilizing any molds or templates for shaping the appliance, thus reducing the number of manufacturing steps involved and improving the resolution and accuracy of the final appliance geometry. Additionally, direct fabrication permits precise control over the three-dimensional geometry of the appliance, such as the appliance thickness. Complex structures and/or auxiliary components can be formed integrally as a single piece with the appliance shell in a single manufacturing step, rather than being added to the shell in a separate manufacturing step. In some embodiments, direct fabrication is used to produce appliance geometries that would be difficult to create using alternative manufacturing techniques, such as appliances with very small or fine features, complex geometric shapes, undercuts, interproximal structures, shells with variable thicknesses, and/or internal structures (e.g., for improving strength with reduced weight and material usage). For example, in some embodiments, the direct fabrication approaches herein permit fabrication of an orthodontic appliance with feature sizes of less than or equal to about 5 μm, or within a range from about 5 μm to about 50 μm, or within a range from about 20 μm to about 50 μm.

The direct fabrication techniques described herein can be used to produce appliances with substantially isotropic material properties, e.g., substantially the same or similar strengths along all directions. In some embodiments, the direct fabrication approaches herein permit production of an orthodontic appliance with a strength that varies by no more than about 25%, about 20%, about 15%, about 10%, about 5%, about 1%, or about 0.5% along all directions. Additionally, the direct fabrication approaches herein can be used to produce orthodontic appliances at a faster speed compared to other manufacturing techniques. In some embodiments, the direct fabrication approaches herein allow for production of an orthodontic appliance in a time interval less than or equal to about 1 hour, about 30 minutes, about 25 minutes, about 20 minutes, about 15 minutes, about 10 minutes, about 5 minutes, about 4 minutes, about 3 minutes, about 2 minutes, about 1 minutes, or about 30 seconds. Such manufacturing speeds allow for rapid “chair-side” production of customized appliances, e.g., during a routine appointment or checkup.

In some embodiments, the direct fabrication methods described herein implement process controls for various machine parameters of a direct fabrication system or device in order to ensure that the resultant appliances are fabricated with a high degree of precision. Such precision can be beneficial for ensuring accurate delivery of a desired force system to the teeth in order to effectively elicit tooth movements. Process controls can be implemented to account for process variability arising from multiple sources, such as the material properties, machine parameters, environmental variables, and/or post-processing parameters.

In properties may vary depending on the properties of raw materials, purity of raw materials, and/or process variables during mixing of the raw materials. In many embodiments, compositions or other materials for direct fabrication are manufactured with tight process control to ensure little variability in photo-characteristics, material properties (e.g., viscosity, surface tension), physical properties (e.g., modulus, strength, elongation) and/or thermal properties (e.g., glass transition temperature, heat deflection temperature). Process control for a material manufacturing process can be achieved with screening of raw materials for physical properties and/or control of temperature, humidity, and/or other process parameters during the mixing process. By implementing process controls for the material manufacturing procedure, reduced variability of process parameters and more uniform material properties for each batch of material can be achieved. Residual variability in material properties can be compensated with process control on the machine, as discussed further herein.

Machine parameters can include curing parameters. For digital light processing (DLP)-based curing systems, curing parameters can include power, curing time, and/or grayscale of the full image. For laser-based curing systems, curing parameters can include power, speed, beam size, beam shape and/or power distribution of the beam. For printing systems, curing parameters can include material drop size, viscosity, and/or curing power. These machine parameters can be monitored and adjusted on a regular basis (e.g., some parameters at every 1-x layers and some parameters after each build) as part of the process control on the fabrication machine. Process control can be achieved by including a sensor on the machine that measures power and other beam parameters every layer or every few seconds and automatically adjusts them with a feedback loop. For DLP machines, gray scale can be measured and calibrated at the end of each build. In addition, material properties and/or photo-characteristics can be provided to the fabrication machine, and a machine process control module can use these parameters to adjust machine parameters (e.g., power, time, gray scale, etc.) to compensate for variability in material properties. By implementing process controls for the fabrication machine, reduced variability in appliance accuracy and residual stress can be achieved.

In many embodiments, environmental variables (e.g., temperature, humidity, Sunlight or exposure to other energy/curing source) are maintained in a tight range to reduce variable in appliance thickness and/or other properties. Optionally, machine parameters can be adjusted to compensate for environmental variables.

In many embodiments, post-processing of appliances includes cleaning, post-curing, and/or support removal processes. Relevant post-processing parameters can include purity of cleaning agent, cleaning pressure and/or temperature, cleaning time, post-curing energy and/or time, and/or consistency of support removal process. These parameters can be measured and adjusted as part of a process control scheme. In addition, appliance physical properties can be varied by modifying the post-processing parameters. Adjusting post-processing machine parameters can provide another way to compensate for variability in material properties and/or machine properties.

### V. Printed Materials

Further provided herein are devices printed with compositions and methods of the present disclosure. In many cases, the printed device comprises a digital material, which can comprise multiple compositions arranged in discrete segments or layers, and which offer complementary physical properties that enhance performance beyond what would be possible with any one of the individual constituent compositions.

In some cases, a printed device of the present disclosure comprises a first cured composition (e.g., the matrix in a cured form) and a second cured composition (e.g., the ink in a cured form) coating at least a portion of the cured first composition. In some cases, the printed device is a medical device. In some cases, the medical device is an orthodontic appliance, a suture, an implant, a joint replacement, a gauze, a graft material, an augmentation material, or a combination thereof. In some cases, the medical device is an orthodontic appliance. In some cases, the orthodontic appliance is an orthodontic aligner, an orthodontic expander or an orthodontic spacer.

In some cases, the cured first composition and the cured second composition each comprise a thickness of between about 5 and about 250 microns (μm). In some cases, the cured first composition comprises a thickness of between about 100 and 2000 microns. In some cases, the cured first composition comprises a thickness of between about 200 and about 1200 microns. In some cases, the cured second composition comprises a thickness of between about 5 and about 500 microns. In some cases, the cured second composition comprises a thickness of between about 25 and about 250 microns. In some cases, the cured second composition comprises a thickness of between about 10 and about 150 microns. In some cases, the cured second composition comprises a thickness of between about 10 and about 50 microns. In some cases, the cured second composition comprises a thickness of between about 5 and about 30 microns. In many cases, a thickness of the cured second composition is at most 300 microns (μm). In some cases, the thickness of the cured second composition is at most 250 microns. In some cases, the thickness of the cured second composition is at most 200 microns. In some cases, the thickness of the cured second composition is at most 150 microns. In some cases, the thickness of the cured second composition is at most 100 microns. In some cases, the thickness of the cured second composition is at most 75 microns. In some cases, the thickness of the cured second composition is at most 50 microns. In some cases, the thickness of the cured second composition is at most 40 microns. In some cases, the thickness of the cured second composition is at most 30 microns. In some cases, the thickness of the cured second composition is at most 25 microns. In some cases, the thickness of the cured second composition is at most 20 microns. In some cases, the thickness of the cured second composition is at most 15 microns. In some cases, the thickness of the cured second composition is at most 10 microns.

In some cases, the cured second composition is disposed as a dot, a linear segment, a layer, layers, or a combination thereof on the cured first composition. In some cases, the cured second composition is disposed with a printing resolution of at most about 500 microns, at most about 300 microns, at most about 200 microns, at most about 150 microns, at most about 100 microns, at most about 75 microns, or at most about 50 microns (e.g., in a plane or curve perpendicular to the thickness).

In some cases, the cured second composition is a cured thiol-ene composition. In such cases, at least about 50%, at least about 60%, at least about 70%, at least about 80%, at least about 90%, at least about 95%, at least about 98%, or at least about 99% of the bonds between monomeric units are thioether bonds. In many cases, the cured second composition does not contain solid additives, such as nanoparticles, powders, or fibers. In some cases, the cured second composition comprises between about 1 and about 60 percent, between about 5 and about 40 percent, between about 5 and about 30, between about 5 and about 20 percent, between about 5 and about 10 percent, between about 10 and about 50 percent, between about 10 and about 30 percent, between about 10 and about 20 percent, between about 20 and about 40 percent, or between about 25 and about 50 percent crosslinker by weight. In some cases, the cured second composition comprises between about 5 and about 40 percent crosslinker by weight. In some cases, the second cured composition comprises a final storage modulus of between about 120 MPa and 360 MPa; a final loss modulus of between about 40 MPa and about 70 MPa; a maximal shrinkage force of between about 2 N and about 4.5 N; a maximal elongation break of between about 14% and 34%; a mass increase of at most about 32% upon exposure to aqueous or organic solvent; an elastic-modulus of between about 3 MPa and about 6 MPa; a hardness of at between about 0.75 MPa and 2 MPa; or a combination thereof.

In some cases, the cured first composition comprises a polyacrylate, a polymethacrylate, or a combination thereof. In some cases, the cured first composition comprises a polymerizable component as described further herein, such as one or more species of polymerizable compounds of the present disclosure (e.g., 1, 2, 3, or more different species), one or more species of polymerizable monomers (e.g., reactive diluents), one or more photoinitiators, one or more toughness modifiers, one or more viscosity modifiers, one or more glass transition temperature modifiers, one or more crosslinkers, as well as combinations thereof. In some cases, the composition comprises: 20 to 50 wt %, based on the total weight of the composition, of a toughness modifier, wherein the toughness modifier is a polymerizable oligomer having a number average molecular weight of greater than 10 kDa; 5 to 80 wt %, based on the total weight of the composition, of a reactive diluent, wherein the reactive diluent is a polymerizable compound having a molecular weight of 0.1 to 0.5 kDa; and 0.1 to 5 wt %, based on the total weight of the composition, of a photoinitiator; wherein the viscosity of the composition is 1 to 70 Pa*s at 110° C. In some cases, the cured first composition comprises a flexural modulus greater than or equal to 50 MPa, 100 MPa, or 200 MPa; an elastic modulus of greater than or equal to 150 MPa, 250 MPa, 350 MPa, 450 MPa, 550 MPa, or between about 500 and 1000 MPa, or from about 550 to about 800 MPa; an elongation at break greater than or equal to 5% before and after 24 hours in a wet environment at 37° C.; a water uptake of less than 25 wt % when measured after 24 hours in a wet environment at 37° C.; transmission of at least 30% of visible light through the polymeric material after 24 hours in a wet environment at 37° C.; a plurality of polymeric phases, wherein at least one polymeric phase of the one or more polymeric phases has a Tg of at least 60° C., 80° C., 90° C., 100° C., or at least 110° C.; or any combination thereof. In some cases, the cured first composition comprises a flexural modulus between about 50 MPa and about 400 MPa; an elastic modulus of between about 100 and about 800 MPa; an elongation at break between about 3% and about 8% (e.g., before and after 24 hours in a wet environment at 37° C.); a water uptake of between about 20 and about 5 wt % when measured after 24 hours in a wet environment at 37° C.; transmission of between about 30% and about 90% of visible light through the polymeric material after 24 hours in a wet environment at 37° C.; a plurality of polymeric phases, wherein at least one polymeric phase of the one or more polymeric phases has a Tg of at least 60° C., 80° C., 90° C., 100° C., or at least 110° C.; or any combination thereof. In some cases, the matrix further comprises a crosslinking agent, a photoinitiator, a stabilizer, a filler, a dye, or any combination thereof. In some cases, the cured first composition comprises: a flexural modulus between about 50 MPa and about 400 MPa; an elastic modulus of between about 100 and about 800 MPa; an elongation at break between about 3% and about 8%; or any combination thereof.

In some cases, the cured first composition is disposed in a plurality of segments or layers, and wherein the cured thiol-ene composition is intercalated between at least a subset of the plurality of segments or layers of the cured first composition. In some cases, the cured second composition is adherent to the cured first composition. In some cases, the cured second composition adheres at least two of the plurality of segments or layers of the cured first composition.

### VI. Medical Devices and Uses Thereof

The present disclosure provides objects such as medical devices that comprise a polymeric material generated by a method of the present disclosure. As described herein, such polymeric material can comprise a component or formulation of the present disclosure. For example, the polymeric material may comprise, incorporated in its polymeric structure, one or more species of polymerizable compound(s) of this disclosure, e.g., compounds according to formulas (I)-(V) and (VII). The polymeric material may also comprise a solid material. In various cases, the device can be a medical device. In some cases, the medical device can be an orthodontic appliance, a suture, an implant, a joint replacement, a graft material, an augmentation material, a prosthetic material or a combination thereof.

The medical device can be an orthodontic appliance. The orthodontic appliance can be an orthodontic aligner, an orthodontic expander or an orthodontic spacer. In some cases, the polymerizable compounds according to the present disclosure can be used as components for viscous or highly viscous photo-curable compositions and can result in polymeric materials that can have favorable thermomechanical properties as described herein (e.g., stiffness, stress remaining, etc.) for use in orthodontic appliances, for example, for moving one or more teeth of a patient. The thermomechanical properties may be improved by homogenization, dispersal, or patterning of constituents of the photo-curable compositions or materials.

As described herein, the present disclosure provides a method of repositioning a patient's teeth, the method comprising: (i) generating a treatment plan for the patient, the plan comprising a plurality of intermediate tooth arrangements for moving teeth along a treatment path from an initial tooth arrangement toward a final tooth arrangement; (ii) producing an orthodontic appliance comprising a polymeric material described herein, e.g., a polymeric material that comprises, in a polymerized form, compounds according to Formulas (I)-(V) and (VII) in a homogenous, dispersed, or patterned distribution; and moving on-track, with the orthodontic appliance, at least one of the patient's teeth toward an intermediate tooth arrangement or the final tooth arrangement. Such orthodontic appliance can be produced using processes that include 3D printing, as further described herein. The method of repositioning a patient's teeth can further comprise tracking progression of the patient's teeth along the treatment path after administration of the orthodontic appliance to the patient, the tracking comprising comparing a current arrangement of the patient's teeth to a planned arrangement of the patient's teeth. In such instances, greater than 60% of the patient's teeth can be on track with the treatment plan after 2 weeks of treatment. In some instances, the orthodontic appliance has a retained repositioning force to the at least one of the patient's teeth after 2 days that is at least 10%, at least 20%, at least 30%, at least 40%, at least 50%, at least 60%, or at least 70% of repositioning force initially provided to the at least one of the patient's teeth.

In certain aspects, the present disclosure provides an orthodontic appliance comprising a crosslinked polymer described herein. The orthodontic appliance may be an aligner, expander or spacer. In some embodiments, the orthodontic appliance comprises a plurality of tooth receiving cavities configured to reposition teeth from a first configuration toward a second configuration. In some embodiments, the orthodontic appliance is one of a plurality of orthodontic appliances configured to reposition the teeth from an initial configuration toward a target configuration, optionally according to a treatment plan.

As used herein, the terms “rigidity” and “stiffness” can be used interchangeably, as are the corresponding terms “rigid” and “stiff” As used herein a “plurality of teeth” encompasses two or more teeth.

In many embodiments, one or more posterior teeth comprises one or more of a molar, a premolar or a canine, and one or more anterior teeth comprising one or more of a central incisor, a lateral incisor, a cuspid, a first bicuspid or a second bicuspid.

In some embodiments, the compositions and methods described herein can be used to couple groups of one or more teeth to each other. The groups of one or more teeth may comprise a first group of one or more anterior teeth and a second group of one or more posterior teeth. The first group of teeth can be coupled to the second group of teeth with the polymeric shell appliances as disclosed herein.

The embodiments disclosed herein are well suited for moving one or more teeth of the first group of one or more teeth or moving one or more of the second group of one or more teeth, and combinations thereof.

The embodiments disclosed herein are well suited for combination with one or more known commercially available tooth moving components such as attachments and polymeric shell appliances. In many embodiments, the appliance and one or more attachments are configured to move one or more teeth along a tooth movement vector comprising six degrees of freedom, in which three degrees of freedom are rotational and three degrees of freedom are translation.

The present disclosure provides orthodontic systems and related methods for designing and providing improved or more effective tooth moving systems for eliciting a desired tooth movement and/or repositioning teeth into a desired arrangement.

Although reference is made to an appliance comprising a polymeric shell appliance, the embodiments disclosed herein are well suited for use with many appliances that receive teeth, for example appliances without one or more of polymers or shells. The appliance can be fabricated with one or more of many materials such as metal, glass, reinforced fibers, carbon fiber, composites, reinforced composites, aluminum, biological materials, and combinations thereof, for example. In some cases, the reinforced composites can comprise a polymer matrix reinforced with ceramic or metallic particles, for example. The appliance can be shaped in many ways, such as with thermoforming or direct fabrication as described herein, for example. Alternatively, or in combination, the appliance can be fabricated with machining such as an appliance fabricated from a block of material with computer numeric control machining. In some cases, the appliance is fabricated using a polymerizable compound according to the present disclosure, for example, using the monomers as reactive diluents for curable compositions.

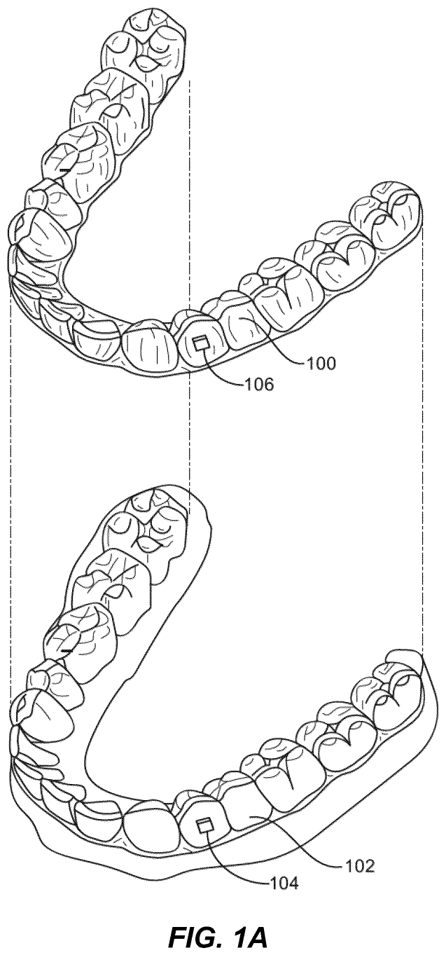

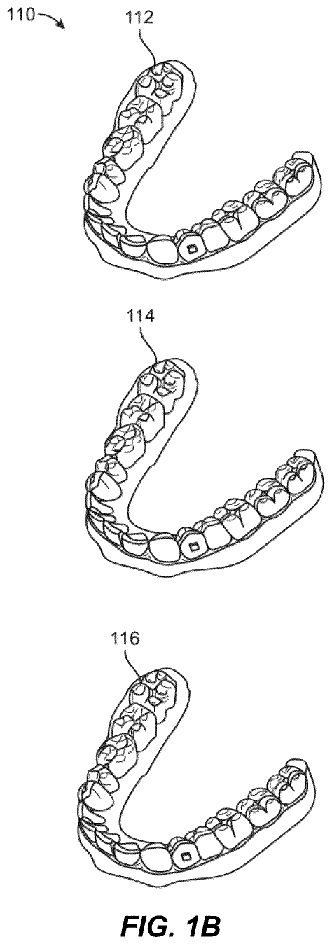

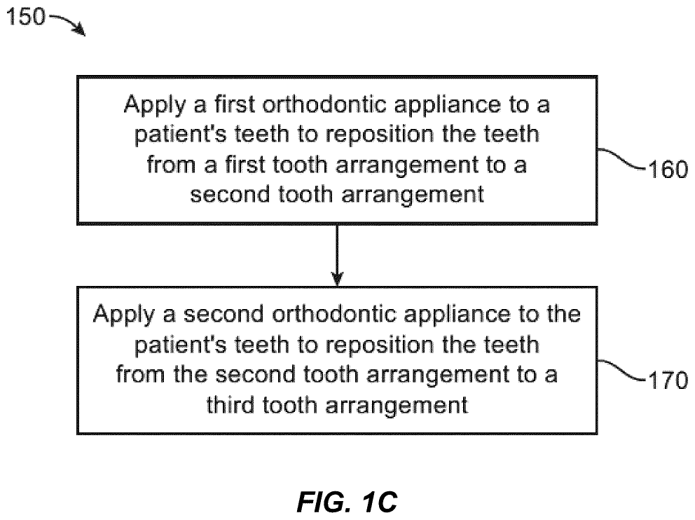

Turning now to the drawings, in which like numbers designate like elements in the various figures, FIG. 1A illustrates an exemplary tooth repositioning appliance or aligner 100 that can be worn by a patient in order to achieve an incremental repositioning of individual teeth 102 in the jaw. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. In some embodiments, a physical appliance is directly fabricated, e.g., using rapid prototyping fabrication techniques, from a digital model of an appliance. An appliance can fit over all teeth present in an upper or lower jaw, or less than all of the teeth. The appliance can be designed specifically to accommodate the teeth of the patient (e.g., the topography of the tooth-receiving cavities matches the topography of the patient's teeth) and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some cases, some, most, or even all of the teeth will be repositioned at some point during treatment. Teeth that are moved can also serve as a base or anchor for holding the appliance as it is worn by the patient. Typically, no wires or other means will be provided for holding an appliance in place over the teeth. In some cases, however, it may be desirable or necessary to provide individual attachments or other anchoring elements 104 on teeth 102 with corresponding receptacles or apertures 106 in the appliance 100 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the url “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 1B illustrates a tooth repositioning system 110 including a plurality of appliances 112, 114, 116. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 110 can include a first appliance 112 corresponding to an initial tooth arrangement, one or more intermediate appliances 114 corresponding to one or more intermediate arrangements, and a final appliance 116 corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages Likewise, an initial tooth arrangement can be any initial arrangement for the patient's teeth that is followed by one or more incremental repositioning stages.

FIG. 1C illustrates a method 150 of orthodontic treatment using a plurality of appliances, in accordance with embodiments. The method 150 can be practiced using any of the appliances or appliance sets described herein. In step 160, a first orthodontic appliance is applied to a patient's teeth in order to reposition the teeth from a first tooth arrangement to a second tooth arrangement. In step 170, a second orthodontic appliance is applied to the patient's teeth in order to reposition the teeth from the second tooth arrangement to a third tooth arrangement. The method 150 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

The configuration of the orthodontic appliances herein can be determined according to a treatment plan for a patient, e.g., a treatment plan involving successive administration of a plurality of appliances for incrementally repositioning teeth. Computer-based treatment planning and/or appliance manufacturing methods can be used in order to facilitate the design and fabrication of appliances. For instance, one or more of the appliance components described herein can be digitally designed and fabricated with the aid of computer-controlled manufacturing devices (e.g., computer numerical control (CNC) milling, computer-controlled rapid prototyping such as 3D printing, etc.). The computer-based methods presented herein can improve the accuracy, flexibility, and convenience of appliance fabrication.

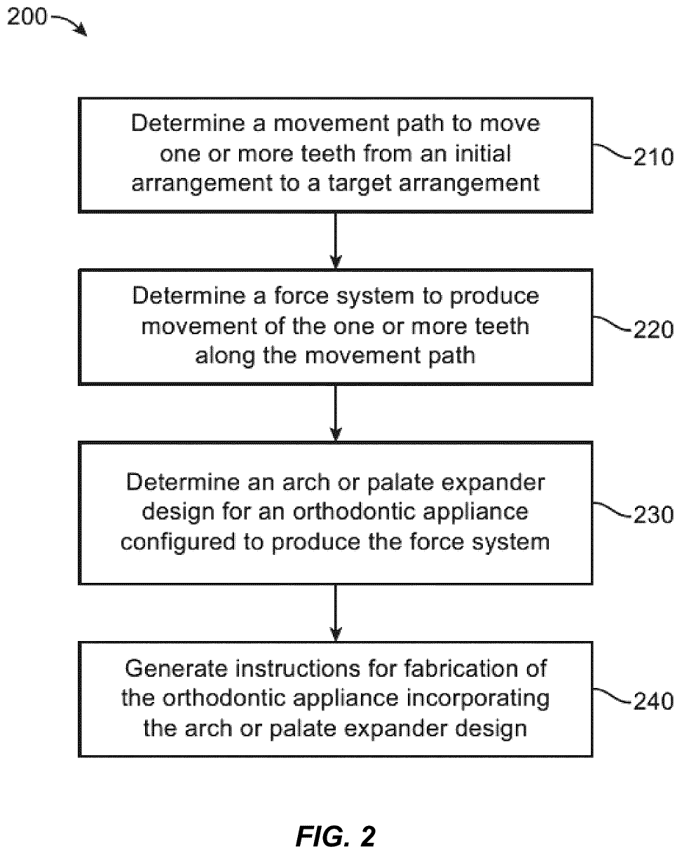

FIG. 2 illustrates a method 200 for designing an orthodontic appliance to be produced by direct fabrication, in accordance with embodiments. The method 200 can be applied to any embodiment of the orthodontic appliances described herein. Some or all of the steps of the method 200 can be performed by any suitable data processing system or device, e.g., one or more processors configured with suitable instructions.

In step 210, a movement path to move one or more teeth from an initial arrangement to a target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. From the obtained data, a digital data set can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, including measured or extrapolated hidden surfaces and root structures, as well as surrounding bone and soft tissue.

The target arrangement of the teeth (e.g., a desired and intended end result of orthodontic treatment) can be received from a clinician in the form of a prescription, can be calculated from basic orthodontic principles, and/or can be extrapolated computationally from a clinical prescription. With a specification of the desired final positions of the teeth and a digital representation of the teeth themselves, the final position and surface geometry of each tooth can be specified to form a complete model of the tooth arrangement at the desired end of treatment.

Having both an initial position and a target position for each tooth, a movement path can be defined for the motion of each tooth. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In step 220, a force system to produce movement of the one or more teeth along the movement path is determined. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as Xray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be measured, or input by a treating professional. In other embodiments, the treating professional can select an appropriate treatment based on physiological characteristics of the patient. For example, the properties of the palate may also be estimated based on factors such as the patient's age—for example, young juvenile patients will typically require lower forces to expand the suture than older patients, as the suture has not yet fully formed.

In step 230, an arch or palate expander design for an orthodontic appliance configured to produce the force system is determined. Determination of the arch or palate expander design, appliance geometry, material composition, and/or properties can be performed using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA(Abaqus) software products from Dassault Systemes of Waltham, MA.

Optionally, one or more arch or palate expander designs can be selected for testing or force modeling. As noted above, a desired tooth movement, as well as a force system required or desired for eliciting the desired tooth movement, can be identified. Using the simulation environment, a candidate arch or palate expander design can be analyzed or modeled for determination of an actual force system resulting from use of the candidate appliance. One or more modifications can optionally be made to a candidate appliance, and force modeling can be further analyzed as described, e.g., in order to iteratively determine an appliance design that produces the desired force system.

In step 240, instructions for fabrication of the orthodontic appliance incorporating the arch or palate expander design are generated. The instructions can be configured to control a fabrication system or device in order to produce the orthodontic appliance with the specified arch or palate expander design. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by thermoforming.

Method 200 may comprise additional steps: 1) The upper arch and palate of the patient is scanned intraorally to generate three dimensional data of the palate and upper arch; 2) The three dimensional shape profile of the appliance is determined to provide a gap and teeth engagement structures as described herein.

Although the above steps show a method 200 of designing an orthodontic appliance in accordance with some embodiments, a person of ordinary skill in the art will recognize some variations based on the teaching described herein. Some of the steps may comprise sub-steps. Some of the steps may be repeated as often as desired. One or more steps of the method 200 may be performed with any suitable fabrication system or device, such as the embodiments described herein. Some of the steps may be optional, and the order of the steps can be varied as desired.

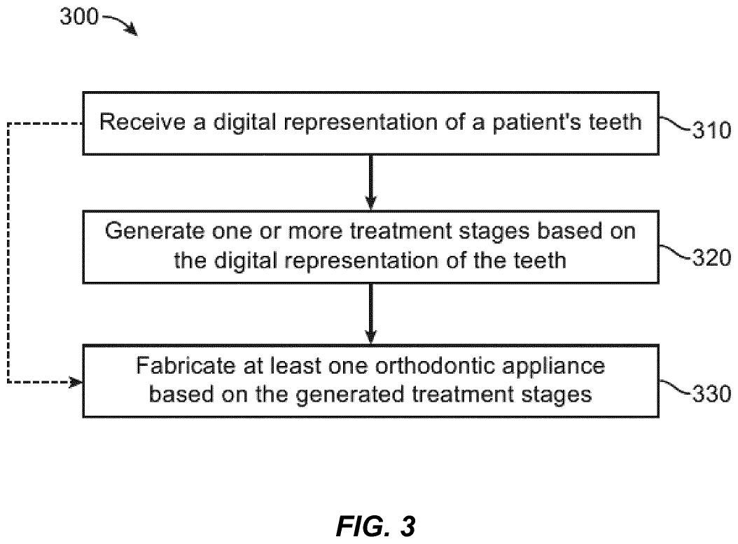

FIG. 3 illustrates a method 300 for digitally planning an orthodontic treatment and/or design or fabrication of an appliance, in accordance with embodiments. The method 300 can be applied to any of the treatment procedures described herein and can be performed by any suitable data processing system.

In step 310, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In step 320, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In step 330, at least one orthodontic appliance is fabricated based on the generated treatment stages. For example, a set of appliances can be fabricated, each shaped according a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired.

In some instances, staging of various arrangements or treatment stages may not be necessary for design and/or fabrication of an appliance. As illustrated by the dashed line in FIG. 3, design and/or fabrication of an orthodontic appliance, and perhaps a particular orthodontic treatment, may include use of a representation of the patient's teeth (e.g., receive a digital representation of the patient's teeth 310), followed by design and/or fabrication of an orthodontic appliance based on a representation of the patient's teeth in the arrangement represented by the received representation.

### VII. Experimental Methods

All chemicals were purchased from commercial sources and were used without further purification, unless otherwise stated.

1H NMR and 13C NMR spectra were recorded on a BRUKER AC-E-200 FT-NMR spectrometer or a BRUKER Advance DRX-400 FT-NMR spectrometer. The chemical shifts are reported in ppm (s: singlet, d: doublet, t: triplet, q: quartet, m: multiplet). The solvents used were deuterated chloroform (CDCl₃, 99.5% deuteration) and deuterated DMSO (d6 DMSO, 99.8% deuteration).

In some embodiments, the stress relaxation of a material or device can be measured by monitoring the time-dependent stress resulting from a steady strain. The extent of stress relaxation can also depend on the temperature, relative humidity and other applicable conditions (e.g., presence of water). In embodiments, the test conditions for stress relaxation are a temperature of 37±2° C. at 100% relative humidity or a temperature of 37±2° C. in water.

The dynamic viscosity of a fluid indicates its resistance to shearing flows. The SI unit for dynamic viscosity is the Poiseuille (Pa·s). Dynamic viscosity is commonly given in units of centipoise, where 1 centipoise (cP) is equivalent to 1 mPa·s. Kinematic viscosity is the ratio of the dynamic viscosity to the density of the fluid; the SI unit is m²/s. Devices for measuring viscosity include viscometers and rheometers. For example, an MCR 301 rheometer from Anton Paar may be used for rheological measurement in rotation mode (PP-25, 50 s-1, 50-115° C., 3° C./min).

Determining the water content when fully saturated at use temperature can comprise exposing the polymeric material to 100% humidity at the use temperature (e.g., 40° C.) for a period of 24 hours, then determining water content by methods known in the art, such as by weight.

In some embodiments, the presence of a crystalline phase and an amorphous phase provide favorable material properties to the polymeric materials. Property values of the cured polymeric materials can be determined, for example, by using the following methods:
stress relaxation properties can be assessed using an RSA-G2 instrument from TA Instruments, with a 3-point bending, according to ASTM D790; for example, stress relaxation can be measured at 30° C. and submerged in water, and reported as the remaining load after 24 hours, as either the percent (%) of initial load, and/or in MPa;
storage modulus can be measured at 37° C. and is reported in MPa;
Tg of the cured polymeric material can be assessed using dynamic mechanical analysis (DMA) and is provided herein as the tan δ peak;
tensile modulus, tensile strength, elongation at yield and elongation at break can be assessed according to ISO 527-2 5B; and tensile strength at yield, elongation at break, tensile strength, and Young's modulus can be assessed according to ASTM D1708.

### EXAMPLES

The specific compositions, synthesis, formulations, and descriptions of any of the materials, devices, systems, and components thereof, of the present disclosure can be readily varied depending upon the intended application, as will be apparent to those of skill in the art in view of the disclosure herein. Moreover, it is understood that the examples and aspects described herein are for illustrative purposes only and that various modifications or changes in light thereof can be suggested to persons skilled in the art and are included within the spirit and purview of this application and scope of the appended claims. Numerous different combinations of aspects described herein are possible, and such combinations are considered part of the present disclosure. In addition, all features discussed in connection with any one aspect herein can be readily adapted for use in other aspects herein. The use of different terms or reference numerals for similar features in different aspects does not necessarily imply differences other than those expressly set forth. Accordingly, the present disclosure is intended to be described solely by reference to the appended claims, and not limited to the aspects disclosed herein.

### Example 1

Synthesis of a Digital Material Including Multilayers

A digital material of the present disclosure included the matrix as a first layer and the ink as a second layer chemically bonded on the first layer that increased toughness of brittleness of the matrix. For example, the hard matrix layers were manufactured by using stereolithography (SLA) and the second layer of the ink (or digital toughening agent) layers were formed by inkjet printing. Such a tandem technique simultaneously benefited from high resolution of the lithography and thin-layer deposition capabilities of the inkjet printing.

The hard matrix formulation included a monomer (e.g., 40 wt % homosalate methacrylate, HSMA), a Tg modifier (e.g., 30 wt % LPU624), a toughness modifier (30 wt % TNM2) a photoinitiator and a photoabsorber disclosed in the present disclosure that were prepared for stereolithography. HSMA had the following structure.

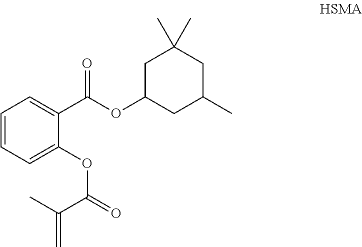 [SMILES: C=C(C)C(=O)Oc1ccccc1C(=O)OC1CC(C)CC(C)(C)C1]

Prior to being poured into the vat, the described formula for the hard matrix was heated, homogenously mixed, and degassed to form an SLA matrix resin. With the help of computer aided manufacturing or computer-aided design (CAM/CAD) software, a UV light source was used to draw a pre-programmed design or shape on to the surface of the photopolymer vat. Photopolymers were sensitive to ultraviolet light, so the resin is photochemically solidified and formed the desired object in a layer by layer fashion. Then, the build platform was lowered one layer and a blade recoats the top of the vat with resin. This process was repeated for each layer until the matrix with a desired thickness is complete. UV light sources included UVA (wavelength about 400 nm to about 320 nm), UVB (about 320 nm to about 290 nm) or UVC (about 290 nm to about 100 nm).

Radical chain growth was the typical mechanism in forming the matrix layer. Chain-growth polymers were formed in a chain-reaction process. The first step involved an initiator to add to a carbon-carbon double bond in the first monomer, which resulted in a reactive intermediate. This intermediate reactive intermediate then went and reacted with a second alkene monomer to yield another reactive intermediate. This process continued to grow the polymer from one monomer to many monomers until the termination step when the radical was consumed.

Once the first matrix layer with the desired thickness was obtained, the second layer of the vet ink layer was formed on the matrix layer by inject printing. The ink layer formulation included a vinyl compound described in the preset disclosure, thiol, a photoinitiator and a stabilizer disclosed in the present disclosure. For example, vinyl ether tri(ethylene glycol) divinyl ether (TEGDVE), an ene-monomer, was mixed with different combinations of the difunctional thiol 2,2′-(ethylenedioxy) diethanoethiol (EDDT) and the trifunctional thiol 1,1,1-tris-(hydroxymethyl)-propane-tris-(3-mercaptopropionate) (TMPMP) to achieve 1:1 molar ratios of ene and thiol groups. The inks included 50% fg % (molar percentage in terms of polymerizable functional groups present) TEGDVE, between 30 and 45 fg % EDDT, and between 5 and 20 fg % TMPMP. Due to the lower viscosity of the inks with 5 and 10 fg % TMPMP, two further formulations were prepared with 1 wt % cellulose acetate butyrate (CAB) to increase viscosity. These formulations were dissolved and homogenized prior to be processed by inkjet printing.

The ink with the above described compositions had a storage stability of at least 20 days at 25° C. and was applied on the first layer of the matrix by an ink jet printer having a resolution of at most 150 microns (μm). The ink was cured by thiol-ene step-growth polymerization and adherent to the first layer. In other words, a thiol-olefin coupling was utilized in a curable thiol-ene composition for polymerization. The ink layer(s) had a thickness of between 5 and 300 μm.

Formation of the soft ink layer on the hard matrix layer was repeatedly carried out to obtain various digital materials having a multiple first matrix layers and the multiple second ink layers. Completed digital materials were washed with a solvent to clean wet resin from their surfaces. In addition, an uncured portion of the first layer of the matrix was removed, so that the digital material with the first and second layer was obtained for various testing and evaluation as described below.

### Example 2

### Physical Characterization of a Curable Composition

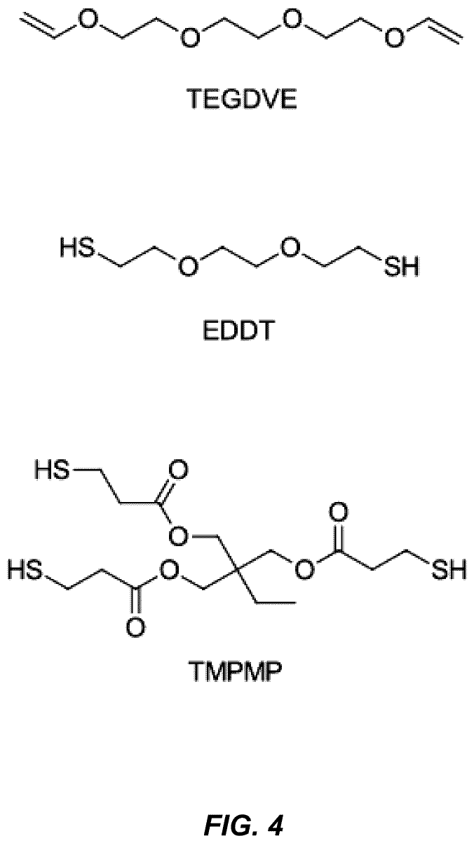

This example covers physical characterizations of multiple curable compositions configured for thiol-ene polymerization. Due to the presence of residual suspended solids, which partially disturbed viscosity measurements, all inks were filtered with a glass frit before characterization. The tested curable compositions are summarized in TABLE 1. Chemical structures of the monomeric species are provided in FIG. 4. All the inks may further include 180 mM pyrogallol and the 30 μm film of the inks were applied with a film applicator frame. The storage modulus (G′) and the loss modulus (G″) of the curable compositions of TABLE 1 are summarized in TABLE 1-1.

TABLE 1

| | TEGDVE | EDDT | TMPMP | CAB | Ivocerin |
|---|---|---|---|---|---|
| Ink Name | [fg %] | [fg %] | [fg %] | [wt %] | [mol %] |
| VET20 | 50 | 30 | 20 | — | 0.5 |
| VET10 | 50 | 40 | 10 | — | 0.5 |
| VET10_1CAB | 50 | 40 | 10 | 1 | 0.5 |
| VET5 | 50 | 45 | 5 | — | 0.5 |
| VET5_1CAB | 50 | 45 | 5 | 1 | 0.5 |

TABLE 1-1

| Ink | G′ [kPa] | G″ [kPa] | Fs [N] |
|---|---|---|---|
| VET20 | 480.5 ± 4.4 | 109.8 ± 0.6 | 6.5 ± 0.2 |
| VET10 | 390.9 ± 3.1 | 73.3 ± 0.1 | 6.6 ± 1.3 |
| VET10_1CAB | 374.3 ± 6.2 | 76.5 ± 0.6 | 6.5 ± 0.4 |
| VET5 | 261.2 ± 2.1 | 48.4 ± 0.1 | 6.6 ± 1.3 |
| VET5_1CAB | 265.1 ± 14.7 | 51.3 ± 0.6 | 6.7 ± 1.1 |

### Surface Tension and Ohnesorge Number

In order to provide insight into its jettability, the viscosity, density, and surface tension were determined for the all the curable composition inks in order to determine its Ohnesorge Number, a unitless parameter which can be calculated as

$$O h = \frac{η}{\sqrt{l * ρ * σ}} ,$$

wherein Oh is Ohnesorge Number, η viscosity in units of mPa*s, ρ is density in units of g/cm³, and σ is surface tension in units of mN/m. The densities of the curable compositions were measured between 22.6 and 23.8° C. with a pycnometer. The surface tension of the curable compositions were measured between 23.8 and 24.7° C. by the pendant drop method. Measured parameters for each curable composition are summarized in TABLE 2.

TABLE 2

| | Viscosity | | | |
|---|---|---|---|---|
| | at 25° C. | Density | Surface Tension | Ohnesorge |
| Ink | [mPa * s] | [g/cm³] | [mN/m] | number |
| VET20 | 7.7 | 1.159 (23.1° C.) | 40.5 ± 0.1 (24.7° C.) | 0.159 |
| VET10 | 6.0 | 1.076 (22.6° C.) | 37.3 ± 0.4 (23.9° C.) | 0.134 |
| VET10_1CAB | 10.0 | 1.070 (23.8° C.) | 38.2 ± 0.4 (23.8° C.) | 0.221 |
| VET5 | 5.1 | 1.064 (22.6° C.) | 35.4 ± 0.4 (23.8° C.) | 0.118 |
| VET5_1CAB | 8.8 | 1.062 (23.6° C.) | 39.0 ± 0.5 (23.8° C.) | 0.193 |

From these measured parameters, the Ohnesorge Number for the curable compositions were calculated to be between 0.018 and 0.221, near the lower limit but still inside of the desired of 0.1 to 1 typically optimal for inkjet compatibility. The curable compositions with lower viscosity were close to this theoretical lower limit, but nonetheless jettable. The density decreased with the reduction of the TMPMP content. The addition of CAB had a minor influence on the density but appeared to increase surface tension. No trend regarding the surface tension was recognizable.

For the viscosity measurements, the rheometer MCR300 from Anton Paar was used and measured with a cone plate (CP25) and a shear rate of 100 s⁻¹ under the temperature range of 25 to 75° C. increasing the temperature by 0.333° C./s.

### Attenuated Total Reflectance Infrared Spectroscopy

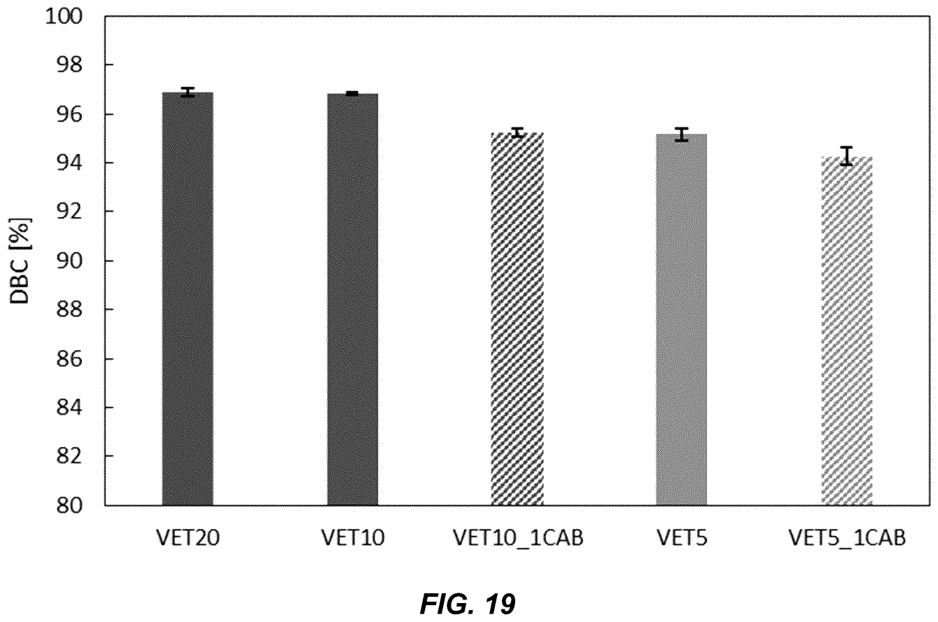

Double bond conversion (DBC) after 5 sec was determined with attenuated total reflectance infrared spectroscopy (ATR-IR) for 30 μm layers of the curable compositions cured with 5 seconds of irradiation with 455 nm LED light. DBC for each of the five curable compositions are summarized in FIG. 19. The ATR-IR measurements revealed double bond conversion rates of greater than 94% for each of the five curable compositions. Lower cross-linker content and the addition of CAB affected marginally lower DBC. By comparison of cured layer appearance, it was noticed that the surface of the formulations VET5 and VET5_1CAB were a bit sticky, likely due to the lower cross-linker content. The other curable compositions yield solid, completely non-sticky surfaces, likely indicating that these curable compositions (VET20, VET10 and VET10_1CAB) polymerized completely under oxygen and did not exhibit any oxygen inhibition effects. Residual peaks from the ATR-IR were mainly attributable to spectral noise, demonstrating that the curable composition exhibited near-complete curing with limited oxygen inhibition.

### RT-NIR-Photorheology Measurements

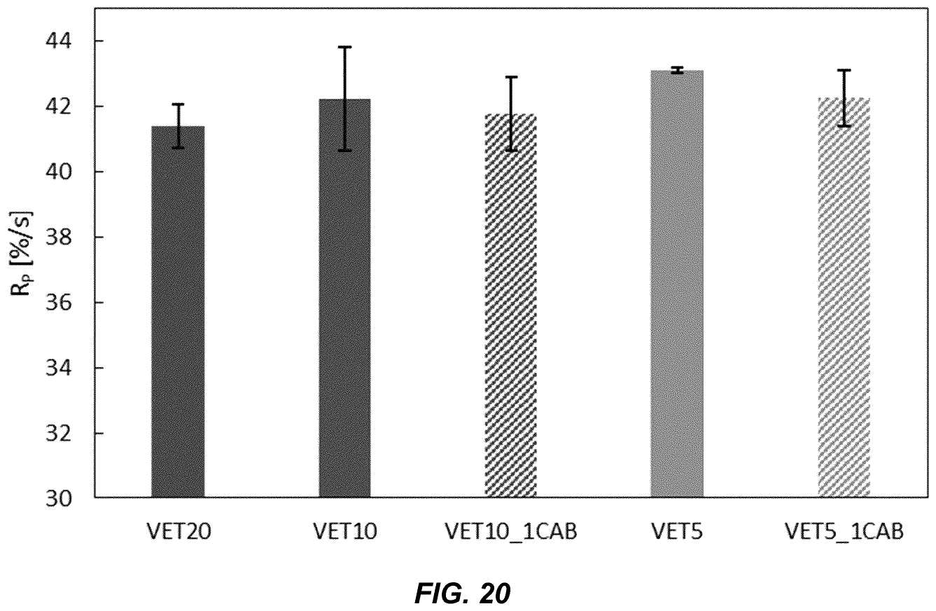

The photoreactivities and mechanical properties of the curable compositions were analyzed with photorheology measurements. The curable compositions were irradiated with a LED with a wavelength of 460 nm and an intensity of 14 mW/cm2 for 300 s while rheology data and near infrared (NIR) spectra were recorded. All formulations reach double bond conversions higher than 99% after the 300 s exposure time. As measure for the photoreactivity, the polymerization rates (R P) are compared in FIG. 20. The curable compositions exhibited similar, fast polymerization rates of between about 41 and 43%/s.

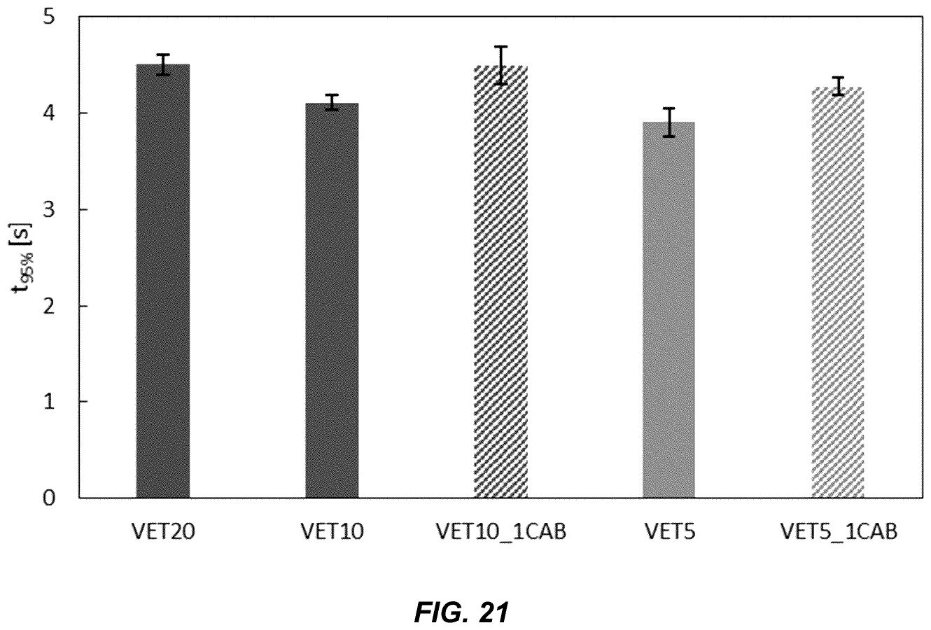

The times required to reach 95% photopolymerization (t_{95%}) determined during the photorheology measurements are summarized in FIG. 21. All curable compositions need less than 5 s to reach 95% of their final double bond conversion. A lower cross-linker content seemed to correlated with slightly shorter t_{95%}. This may have been due to enhanced flexibility enabling greater mobility and conformational freedom of unreacted groups. The addition of CAB slightly reduced reactivity, which was reflected in slightly higher t_{95%} values.

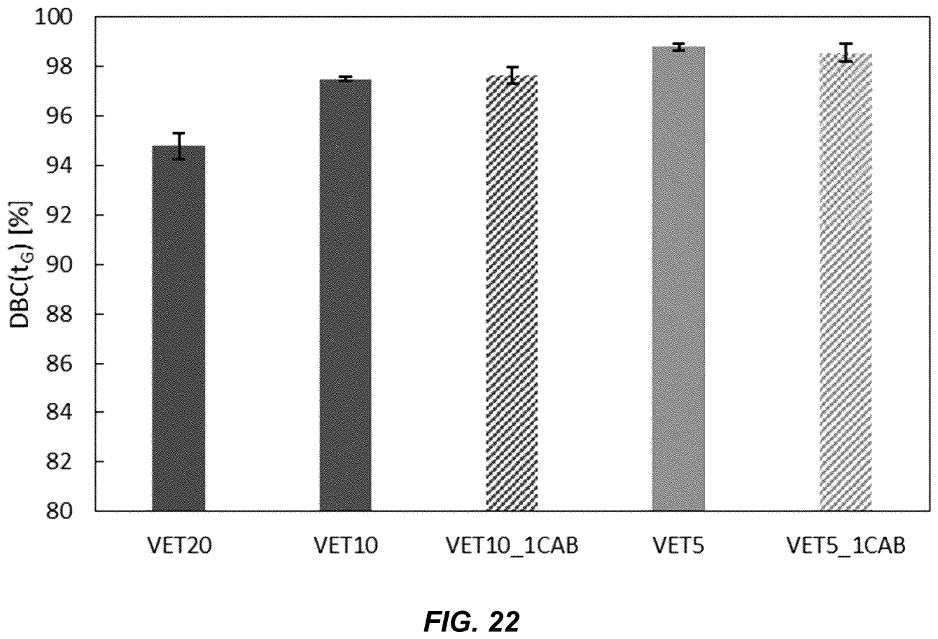

FIG. 22 provides the double bond conversion rates at the gel points (t_{G}) for each curable composition. Since the curable compositions underwent pure step growth polymerization, the gel points, which are defined as intersection of the storage modulus G′ and the loss modulus G″, were shifted to relatively high values. As lower cross-linking density increases the double bond conversion requirement for gel formation, the DBC on the gel point of VET5 is the highest, followed by VET10 and VET20. The additive CAB had a minor influence on this measure.

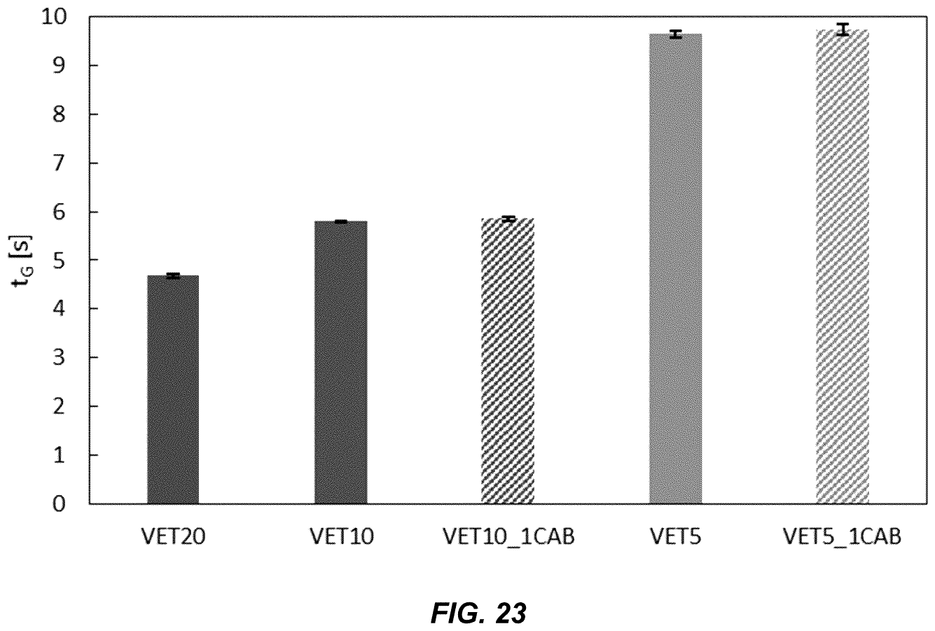

FIG. 23 displays time to reach gel point (t G) for each curable composition. Across the five curable compositions, increased crosslinker content appeared to correlate with faster gelation. While VET20 required only 4.7 s to form a gel, the reduction of the cross-linker TMPMP to 10 fg % delayed the t_{G} to 5.8 s. Further decreasing the cross-linker content to 5 fg % doubled t_{G} to 9.6 s. As with DBC at t_{G}, the addition of CAB minorly influenced t_{G}.

In general, the reactivity of all curable compositions were sufficiently high for inkjet printing, and further exhibited high double bond conversion necessary to form sufficiently cured polymers. Notably, the light intensity of the LED in the printer was higher than that of the photorheology setup (14 mW/cm²), suggesting that exposure time requirements may be lower than suggested by the measurements.

### Dynamic Mechanical Thermal Analysis

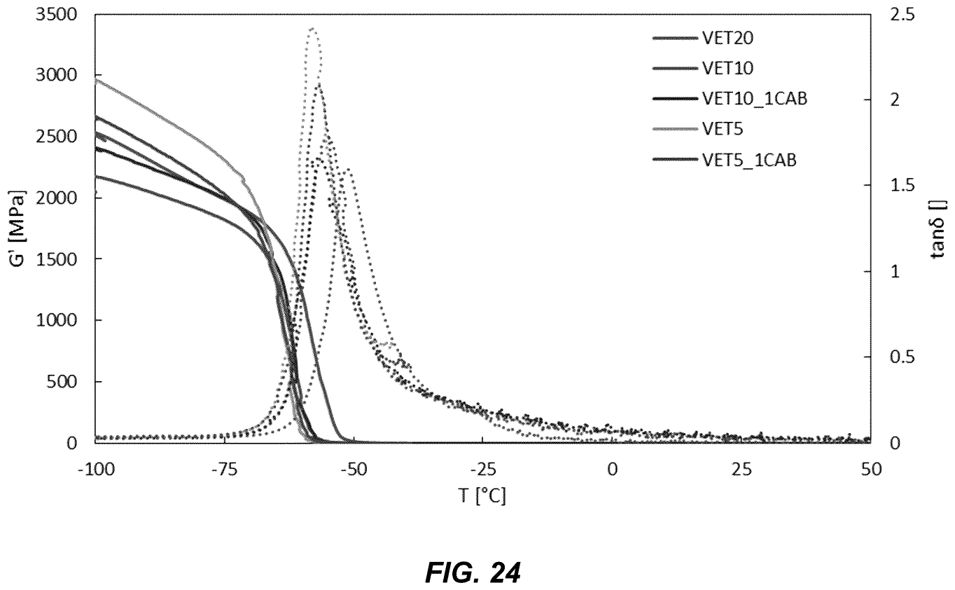

Dynamic mechanical thermal analysis (DMTA) measurements were conducted on the curable compositions to determine their glass transition temperatures (Tg) and the storage moduli at room temperature (G′, 25° C.). UV-cured specimens were oscillated with a frequency of 1 Hz and an amplitude of 1%. The storage modulus G′ and the dissipation factor tanδ are plotted against the temperature and both are plotted in FIG. 24, with storage modulus G′ indicated with solid lines and dissipation factor tanδ indicated with dotted lines.

All curable compositions exhibited low glass transition temperatures of below −50° C. Reducing cross-linker content appeared to decrease the Tg slightly, while no trend was recognizable regarding the addition of CAB. The Tg of VET10_1CAB was lower than that of VET10, while VET5_1CAB exhibited a marginally higher Tg than VET5. The storage moduli of the curable compositions at 100° C. were between 2 and 3 GPa. The storage moduli decreased slightly approaching the Tg's, and just before the Tg's exhibited sharp drops, likely due to the regulated network formed by the step-growth polymerization mechanism. At 25° C. the storage moduli are lower than 2 MPa, explaining why the curable compositions behaved as soft materials near room temperature. Consistent with low network density typically corresponding to softness, VET10 had a lower G′(25° C.) than VET20. This measure could not be determined for VET5 and VET5_1CAB since the samples were sufficiently soft to stretch after the glass transition temperature. However, the storage modulus was already below 1 MPa at −40° C. The results of these analyses are summarized in TABLE 3.

TABLE 3

| Ink | Tg [° C.] | G′ [MPa] | E-Modulus [Mpa] |
|---|---|---|---|
| VET20 | −51.2 | 1.25 | 2.17 ± 0.02 |
| VET10 | −54.8 | 0.7 | 1.33 ± 0.05 |
| VET10_1CAB | −57.3 | 0.73 | 1.31 ± 0.05 |
| VET5 | −58.1 | 0.49 (−40° C.) | 0.28 ± 0.01 |
| VET5_1CAB | −56.8 | 0.73 (−40° C.) | 0.41 ± 0.01 |

### Tensile Tests

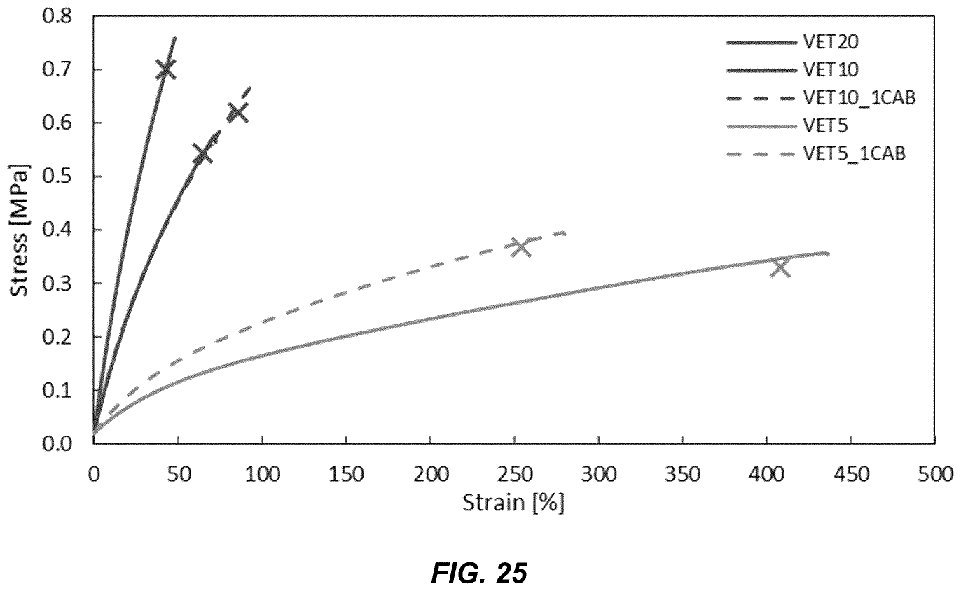

The mechanical properties of the curable compositions were further investigated by tensile tests using a traverse speed of 5 mm/min. FIG. 25 displays a stress strain curve for each curable composition, with the average elongation and stress at break of the four best samples are marked with an X. As expected, the elongation at break increased and the tensile strength decreased with reduced crosslinker content (corresponding to network density). The elongation at break of VET20 was 43%. The reduction of the cross-linker to 10 fg % enhanced this measure to 65%, while further reduction to 5 fg % further increased average elongation at break to 408%. The addition of CAB differently affected the 5% and 10% crosslinker compositions. VET10_1CAB showed a similar curve propagation as VET10, but also exhibited higher tensile strength and elongation at break than the non-CAB-containing curable composition. In contrast, the addition of CAB to VET5 increased the tensile strength but decreased the elongation at break considerably to 254%. The tensile properties were well adjustable by the cross-linker content.

### Swelling Tests

Swelling tests were conducted with a reference matrix containing 30 wt % LPU624, 30 wt % TNM2 and 40 wt % HSMA (Matrix 334). The mass increases of matrix plates swelled for 22h at 50° C. in the curable compositions as well as in the individual components were measured and the results are shown in TABLE 4.

TABLE 4

| Sample Plate | Monomer/Ink | Mass Increase [%] |
|---|---|---|
| Matrix 334 | TEGDVE | 23.5 |
| Matrix 334 | EDDT | 15.5 |
| Matrix 334 | TMPMP | 0.2 |
| Matrix 334 | VET20 | 10.9 |
| Matrix 334 | VET10 | 18.5 |
| Matrix 334 | VET10_1CAB | 22.8 |
| Matrix 334 | VET5 | 22.8 |
| Matrix 334 | VET5_1CAB | 27.1 |

Regarding the individual curable composition constituents, it is noticeable that the vinyl ether TEGDVE swells the matrix the most, followed by the difunctional thiol EDDT, while the trifunctional thiol TMPMP affected minimal mass increases in the reference matrix, likely owing to its higher viscosity. This trend is reflected in the swelling results obtained with the five curable compositions, from which TMPMP content correlated with lower matrix swelling. VET20 lead to the lowest mass increase (10.9%), followed by VET10 (18.5%) and VET5 (22.8%). The addition of CAB increased swelling, despite its simultaneous effect of increasing curable composition viscosity.

### Jetting Experiments

Jetting experiments were conducted using a dropwatcher system with the printhead MH5421F and the head interface board HIB-RH-1280. A single-, double- and triple-pulse waveform was designed to vary the drop size of the curable compositions. Those waveforms were applied on the three curable compositions VET20, VET10 and VET5. The pulse widths were varied based on the viscosities of the analyzed curable compositions. For VET20, the curable composition with the highest viscosity, higher pulse widths were chosen (first pulse: 10 μs, second pulse: 4 μs, third pulse: 6 μs). VET10 was used with lower pulse widths (5.5 μs, 2 μs and 3 μs) and for VET5 those times were further reduced (3 μs, 1 μs and 1.5 μs). After jetting one drop of the respective curable composition through each nozzle of the printhead on an object slide, the drops were irradiated for 5 s with a 455 nm LED light and analyzed by laser scanning microscopy (LSM).

FIG. 26 provides LSM images of the different curable compositions printed with the different waveforms. In this figure, the leftmost column corresponds to single (S) waveform printing, the middle column corresponds to double waveform (D) printing, and the rightmost column corresponds to triple waveform (T) printing; while the top row corresponds to VET20, the middle row corresponds to VET10, and the bottom row corresponds to VET5 curable compositions. As illustrated in this figure, the drop size was increased by using a waveform with higher number of pulses. The single-pulse waveform led to small drops for all curable compositions, while the drops with the double-pulse waveform were significantly increased. The triple-pulse waveform led to some merged drops. These results demonstrate that, depending on the desired curable composition geometry in the digital material, a suitable waveform can be chosen. For drops and lines, the single- or double-pulse waveform can be used, while for full layers the triple- pulse waveform is an additional option to vary the curable composition content in the final material.

Generally, it was shown that all three curable compositions are jettable despite their relatively low viscosities. Therefore, it is not necessary to include a viscosity-enhancing additive, such as cellulose acetate butyrate (CAB) to increase the viscosity. Hence, the curable compositions VET10_1CAB and VET5_1CAB were not further analyzed due to the slightly decreased reactivity and the higher swelling.

### Example 3

### Curable Composition Storage Stabilities

This example covers the storage stability of various curable compositions based on VET20, as outlined and printed in EXAMPLE 2. As many inkjet printers require room temperature or higher than room temperature conditions for printing, suitable curable compositions often need to exhibit high stabilities and low polymerization during storage to ensure that the curable composition would not gel and block a printhead prior to use. The effects of multiple stabilizers, including pyrogallol and MA-154, were tested. MA-154 has the following structure:

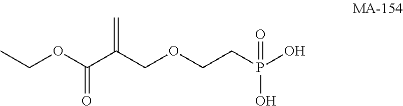

Due to the low viscosities of the curable compositions, viscosity measurements were performed at 25° C. The measurements were performed with a cone plate, a gap of 48 μm and a shear rate of 100/s. The obtained data points also served as starting points for the storage stability tests, for which further viscosity measurements after certain time intervals were conducted. The samples were stored under different conditions (refrigerator, 25° C., 37° C. and 50° C.) to see the influence of the temperature on the storage stability.

To determine whether stabilizers could be used to increase the storage stabilities of the curable compositions, VET20 curable compositions with 9 mM of the stabilizer pyrogallol and either containing or lacking 90 mM of the costabilizer MA-154 were tested for stability. Samples of the resultant curable compositions were stored for 1 day of refrigeration or 50° C. storage. Both the MA-154-containing and MA-154-free compositions gelled after 1 day of 50° C. While neither curable composition gelled during 1 day of refrigerated storage, both curable compositions exhibited increased viscosities, evidencing that some polymerization had already occurred under these conditions. During this timeframe, the MA-154-containing curable composition underwent a greater viscosity increase than the MA-154-free sample. Under refrigeration, the MA-154-containing curable composition fully gelled after 1 week, as compared to about 1 month for the MA-154-free sample.

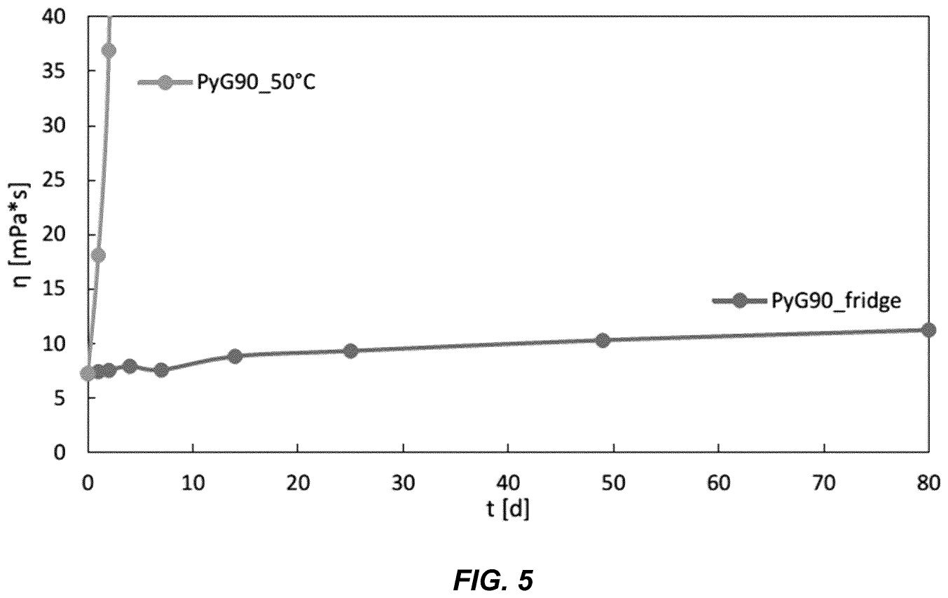

Since the co-stabilizer increased the gelation rate of the VET20 pyrogallol curable compositions, subsequent test-inks were prepared with only pyrogallol. Pyrogallol content was increased to 90 mM and the stability of the curable composition was tested over multiple days of refrigeration or 50° C. storage. The curable composition exhibited an initial viscosity of about 7 mPa*s. As shown in FIG. 5, the curable composition was stable over the first 7 days of refrigerated storage, after which time the viscosity slowly increased, reaching 155% of its initial viscosity after 80 days. Under 50° C. storage, the viscosity of the curable composition increased rapidly, surpassing 2000 mPa*s viscosity after one week and reaching complete gelation during the second week of storage.

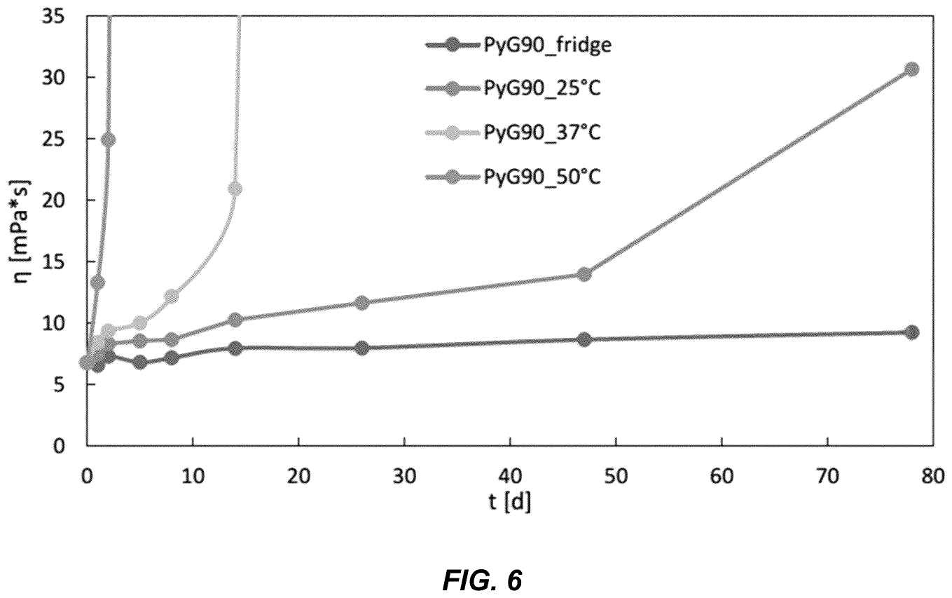

New VET20 curable compositions with 90 mM pyrogallol and lowered photoinitiator concentration (0.5 mol% ivocerin) were prepared. The stabilities of this mixture was tested at 50° C., 37° C., 25° C., and under refrigeration. The results of these analyses are summarized in FIG. 6. Similar to the higher photoinitiator concentration curable composition, the viscosity of the mixture upon formulation (prior to storage) was about 7 mPa*s. The composition exhibited improved stability under refrigerated storage, with a 36% viscosity increase after 78 days. At 25° C. the viscosity increased slowly for the first 8 days, exhibiting only 27% increase in viscosity, followed by an increased rate of gelation. Following 78 days of 25° C., the curable composition had increased in viscosity by 350%. The curable composition exhibited markedly diminished stability at 37° C., with a viscosity increase of 79% after just 8 days and complete gelation by 47 days. The curable composition increased in viscosity and gelled quickly at 50° C.

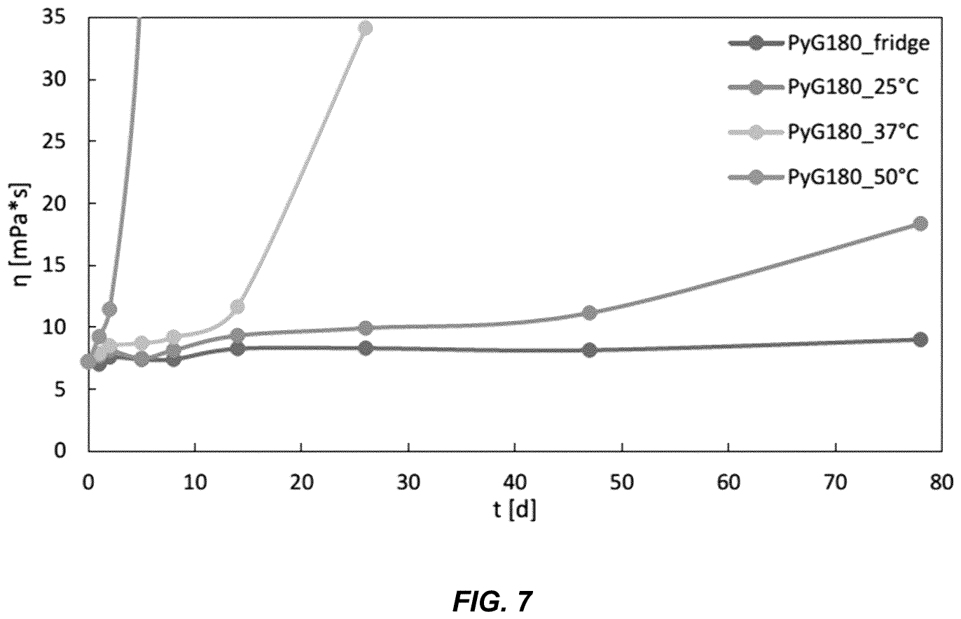

Doubling the amount of pyrogallol (180 mM) in the low photoinitiator curable composition further increased its storage stability. Viscosities of this curable composition following variable temperature storage are shown in FIG. 7. While at 50° C. the curable composition quickly increased in viscosity and gelled within two weeks, at 37° C. the curable composition exhibited slower viscosity increases, increasing in viscosity by about 28% after 8 days, less than 100% after two weeks, and complete gelling after 47 days. Under refrigerated storage, the curable composition was stable over 8 days of storage and exhibited only 25% viscosity increase following 78 days of storage. The curable composition also exhibited a high degree of stability at 25° C., with negligible viscosity increase over 8 days of storage, 55% viscosity increase following 47 days, and 154% viscosity increase following 78 days.

Viscosity changes during variable temperature storage are summarized for the TABLE 1 curable compositions in FIGS. 19-21. Immediately upon formulation (on day 0), the curable compositions exhibited viscosities ranging from 5.1 to 10.0 mPa*s. By reducing the TMPMP content from 20 fg % to 10 and 5 fg %, the viscosity was decreased from 7.7 mPa*s to 6.0 and 5.1 mPa*s. The addition of 1 wt % CAB increased the viscosity again to 10.0 mPa*s for VET10_1CAB and 8.8 mPa*s for VET5_1CAB. The formulations all exhibited stability at refrigerated, room (25° C.) and slightly elevated (37° C.) temperatures, under which conditions the viscosities increased minimally. The stabilities of the curable compositions decreased at 50° C., but nonetheless demonstrated the ability to be stored at this elevated temperature for 2-3 weeks without significant viscosity increase.

It is noticeable that for some curable compositions greater viscosity increases were observed at 25° C. than at higher temperatures. For some curable compositions, the viscosity is already significantly increased after 43 days and all samples are gelled after 89 days. The reason for this discrepancy could have been due to a difference in storage conditions. While the other samples were kept under light exclusion in drying ovens, the samples at 25° C. were stored in partially opaque brown glass vials on a laboratory bench which could not be entirely shielded from ambient light completely. The results suggest that it may be important to store the curable compositions cooled and under complete light exclusion to guarantee storage stability over a long period of time, but that it is also possible to heat them up during printing jobs without risking gelation in the print head.

### Example 4

### Photorheological and Physical Characterizations of a Curable Composition

This example covers a photorheological and physical characterizations of the curable composition which was prepared as outlined in EXAMPLE 2. For photorheological analysis, the curable composition was irradiated with 460 nm LED light for 300 s while rheology data and NIR-spectra were recorded. Following this irradiation period, the curable composition was completely polymerized with a final double bond conversion of greater than 99%. The reactivity of the curable composition was also very high, exhibiting a polymerization rate (Rₚ) of about 41% per second, with a time to 95% polymerization (t_{95%}) of just 4.5 s. Since VET20 undergoes step growth polymerization, the gel point, which is defined as the intersection of the storage modulus (G′) and the loss modulus (G″ is shifted to a very high conversion of 94.8%. This means the time until the gel point is reached (t_{G}) is with 4.7 s even slightly later than the t_{95%}. These values indicate that the reactivity of the VET20 curable composition is very high. Due to the ongoing thiol-ene step growth polymerization, a high double bond conversion could be necessary for this curable composition to receive a sufficient cured polymer. However, the light intensity of the LED in the printer is significantly higher than that of the photorheology setup (14 mW/cm²), while the required exposure time may be lower than the measured 4.7 s, which makes the curable composition well suitable for inkjet printing. The final storage modulus (G′) was measured to be 481±4 MPa, while the final loss modulus was measured to be 110±1 MPa. The maximal shrinkage force was measured as 6.5±0.2 N.

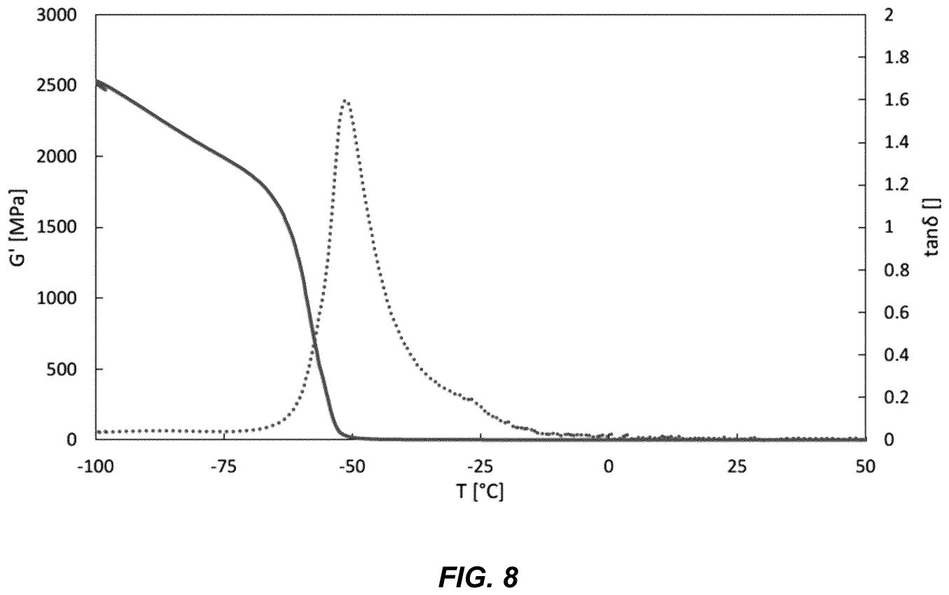

The curable composition was subjected to Dynamic Mechanical Thermal Analysis (DMTA) to provide insight into its glass transition temperature (T_{g}) and its storage modulus. The results of these analyses are provided in FIG. 8, with storage modulus G′ shown with the solid plot and on the leftmost axis, and the dissipation factor (the tangent of the phase angle (δ) between stress and strain, tanδ) shown with the dotted plot and on the rightmost axis. The curable composition exhibited a low glass transition temperature of −51.2° C., which correlates with the maximum of the tanδ-curve. The storage modulus G′ of the curable composition was about 2.5 GPa at −100° C., decreased to 1.7 GPa at −65° C., and dropped precipitously thereafter due to the glass transition. There was an additional small drop at −27° C., which is visible as a shoulder in the tanδ-curve at this temperature. The storage modulus was constant as temperature was increased through higher temperatures, leading to 1.25 MPa at 25° C. Despite the low glass transition temperature, G′ was stable between −20° C. and 50° C.

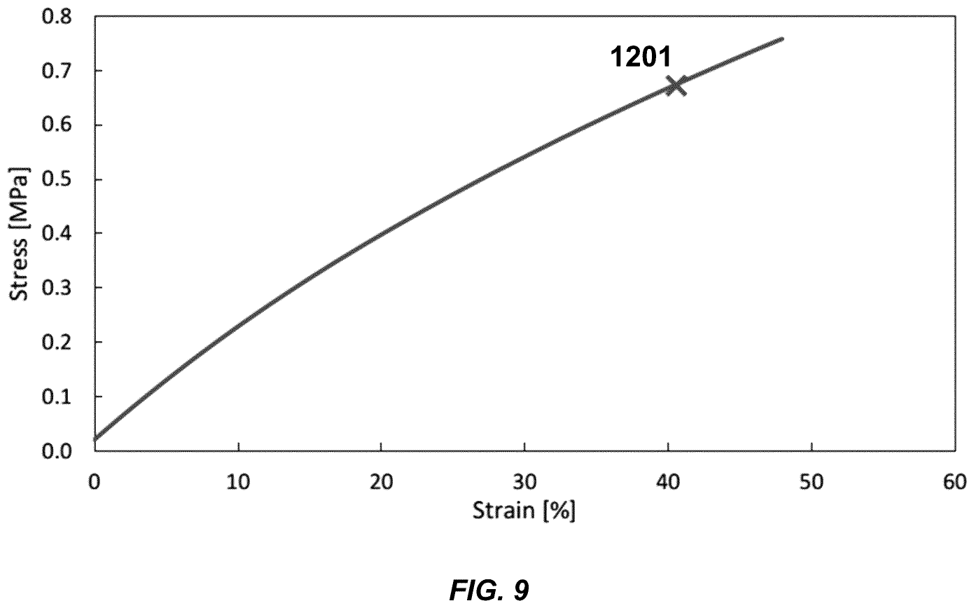

Stress-strain measurements were performed on the cured curable composition to determine its elongation at break. The results of these analyses are summarized in FIG. 9, with strain (%) on the x-axis, stress (MPa) on the y-axis, and the stress at break marked with an X (1201). The curable composition showed a maximum elongation break of 48% and an average value of 40.6%. While this was lower than the estimated value of 75% for this curable composition, the elongation at break was above the target of the final digital material intended to be printed with the curable composition. The curable composition further exhibited elastomeric behavior.

The mass increase of a reference matrix swelled in the individual curable composition components, in the curable composition, and in a reference alternative thiol (1,6-hexanedithiol (HDT)) are summarized in TABLE 5. The developed curable composition effected a mass increase of the matrix of 10.9%. From the analysis of the individual curable composition components, the ene-compound TEGDVE appeared to be mainly responsible for this diffusion, followed by the dithiol EDDT, while the trithiol TMPMP showed very low swelling due to the higher viscosity. The possibility to replace EDDT by another available dithiol (HDT) was discarded since HDT showed significantly higher swelling (27.3%) despite the absence of the polar ether groups. Further tests such as nanoindentation were postulated as potential means for determining whether the diffusion into the matrix was sufficiently low to generate well separated layers.

TABLE 5

| Monomer/Curable Composition | Mass Increase [%] |
|---|---|
| VET20 | 10.9 |
| TEGDVE | 23.5 |
| EDDT | 15.5 |
| TMPMP | 0.2 |
| HDT | 27.3 |

### Example 5

### Printing and Physical Characterization of a Digital Device

A digital material was prepared by printing 50 micron layers a matrix material containing 60 wt % Bomar XR-741MS, 20 wt % HEMA and 20 wt % HSMA (BHH622) with a direct light processing (DLP) printer, interspersed with dots, lines, or layers of TEGDE50EDDT30T20 curable composition (as outlined in EXAMPLE 2) applied with an inkjet printer. Bomar XR-741MS is a difunctional, aliphatic polyester urethane methacrylate but the exact chemical structure is unknown, and HEMA is 2-Hydroxyethyl methacrylate.

Nanoindentation measurements were performed to analyze the separation of the matrix layers and the curable composition layers in the digital materials, with the E-modulus and the hardness determined normal to the individual layers. In addition, the E-modulus and hardness of the pure curable composition and the pure matrix were measured. The curable composition specimen, which was molded and showed no orientation within the sample, was measured 35 times on different areas with a force of 30 μN, indicating an E-modulus of 9.1±0.3 MPa and a hardness of 2.5±0.2 MPa. The pure matrix sample was printed and scanned by conducting five to seven indentations along individual layers (corresponding to the x-direction of the printing process) with a force of 100 μN before moving 5 μm normal to the layers (z-direction of the printing process) and measuring the next five data points. As there were no significant relationships between either E-modulus or hardness and measurement location, the values from each measurement were averaged, yielding an E-modulus of 6.3±0.7 GPa and a hardness of 0.4±0.1 GPa, 700- and 160-times those of the printed curable composition.

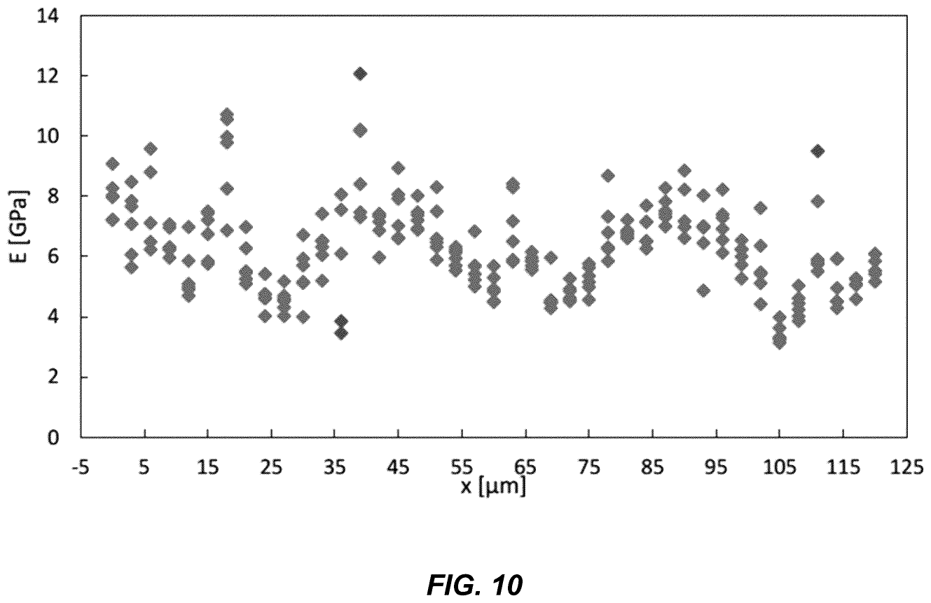

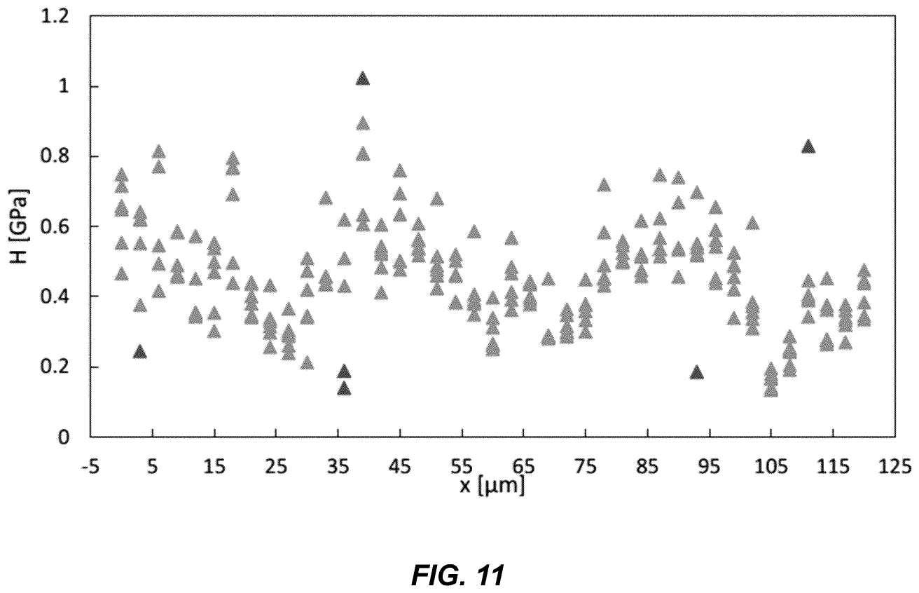

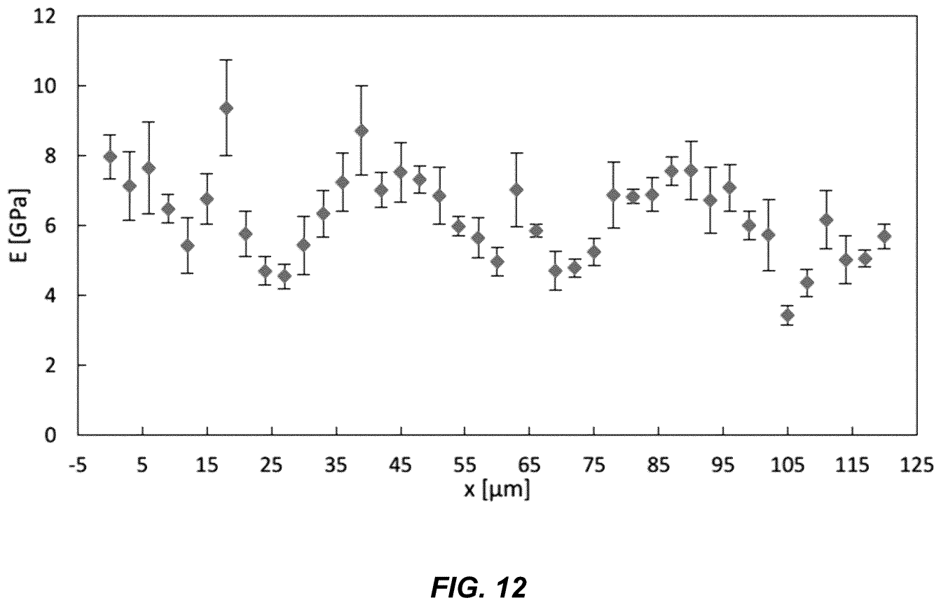

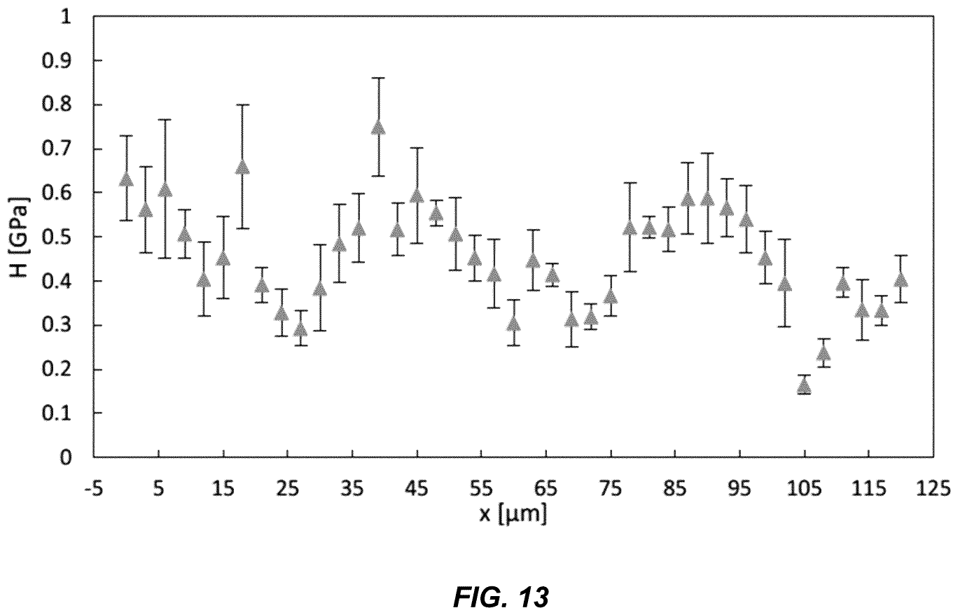

Next, the digital material printed with curable composition layers was analyzed with a similar method as the pure matrix material. All received data points for the E-modulus and hardness are displayed in FIGS. 10-11, respectively. In each of these figures, the red-marked data points were identified as outliners, and the residual points were used to calculate average E-modulus and hardness, as shown in FIGS. 12-13, respectively. Both figures showed similar trends, with layers exhibiting visible minima in E-modulus and hardness every 40 to 45 μm, corresponding approximately to the target layer thickness of 50 μm. Notably, the maxima exhibited higher E-modulus values than the pure matrix, which possibly could have been caused either by the additional irradiation step of the curable composition or by diffusion of additional photoinitiator in the matrix, which could have increased the conversion of the matrix and therefore its E-modulus and hardness. Furthermore, as the hardest and softest layers differed by only a factor of 2, it is unlikely that pure curable composition layers were present, and instead evidence mixing between the curable composition and matrix layers. It is hypothesized that an E-modulus difference of a factor of 5 or more may enhance resistance to crack propagation.

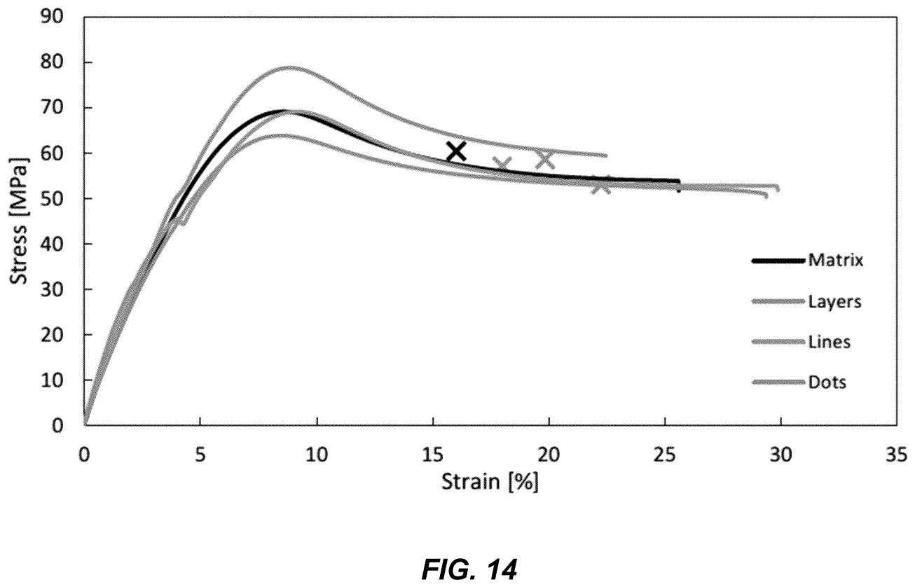

To test the influence of the form of the applied curable composition on the toughness of the digital materials, tensile tests of the pure matrix material BHH622 as well as of the digital materials with different curable composition geometries were conducted. The results of these analyses are shown in FIG. 14 (with elongation at break points indicated with X-marks on the plots), and important values are summarized in TABLE 6, with difference values (Δϵ_{B}, Δσ_{M}, and ΔToughness) relative to the pure matrix material. The average and maximum elongation at break of the matrix BHH622 were measured to be 16% and 25.6%, respectively. All digital materials exhibited higher tensile strengths than the pure matrix material. Nonetheless, higher amounts of curable composition appeared to reduce measured the tensile strengths. This disparity may have been due to the additional irradiation step and/or diffusion in the digital materials, which could have increased matrix conversion. Further to this possibility, the lower tensile strength of the curable composition layer-containing digital material may have been due to increased volume of the lower tensile strength curable composition layers.

TABLE 6

| | ε_{B} | Δε_{B} | σ_{M} | Δσ_{M} | Toughness | ΔToughness |
|---|---|---|---|---|---|---|
| Sample | [%] | [%] | [MPa] | [%] | [MJ/m³] | [%] |
| BHH622 | 16.0 ± 0.1 | — | 73.0 ± 3.6 | — | 8.9 ± 2.3 | — |
| BHH622-Layers | 1.5 ± 0.1 | 39.0 | 66.5 ± 3.5 | −7.7 | 11.7 ± 1.9 | 31.5 |
| BHH622-Lines | 1.7 ± 0.0 | 12.4 | 71.6 ± 1.9 | 1.7 | 9.8 ± 3.3 | 10.0 |
| BHH622-Dots | 1.8 ± 0.0 | 23.9 | 77.3 ± 1.9 | 8.1 | 11.7 ± 1.5 | 31.5 |
| BHH601525 | 1.1 ± 0.3 | | | | | |
| BHH601525-Layers | 0.9 ± 0.3 | −23.2 | 77.3 ± 1.9 | 8.1 | 11.7 ± 1.5 | 31.5 |
| BHH601525-Lines | 1.2 ± 0.2 | 9.8 | 77.3 ± 1.9 | 8.1 | 11.7 ± 1.5 | 31.5 |
| BHH601525-Dots | 1.6 ± 0.2 | 43.7 | 77.3 ± 1.9 | 8.1 | 11.7 ± 1.5 | 31.5 |

The results show that the elongation at break and the toughness of the digital materials could be increased, despite the E-modulus difference of only a factor of 2 (instead of the desired factor of 5). The highest tensile strength values corresponded to the digital material with curable composition lines, which exhibited an elongation at break is in the range of 10 and 35% and a toughness of 15.9 MJ/m³. The digital materials with layers and dots exhibited the highest toughness increases of 31.5%, corresponding to the greatest increase of the elongation at break.

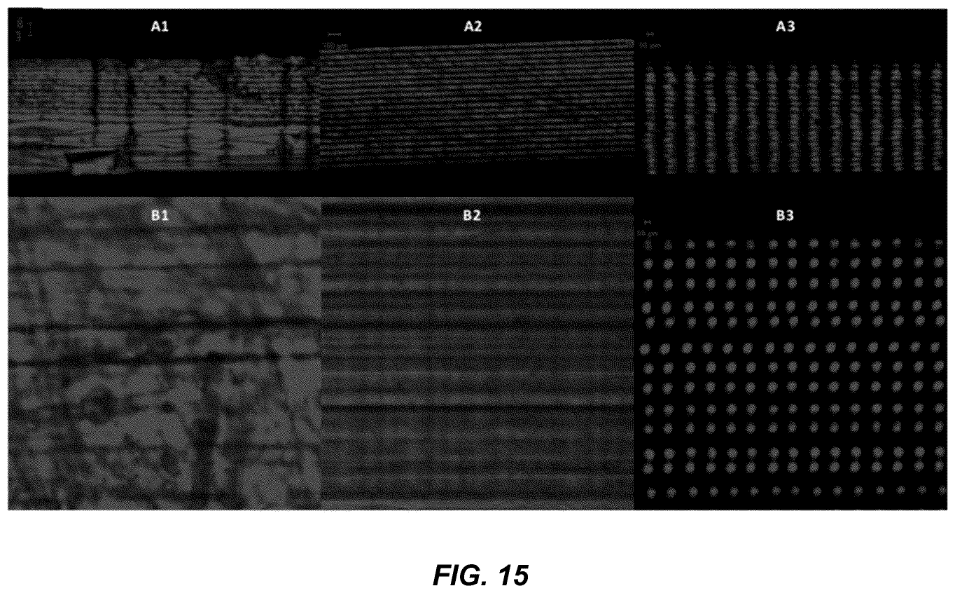

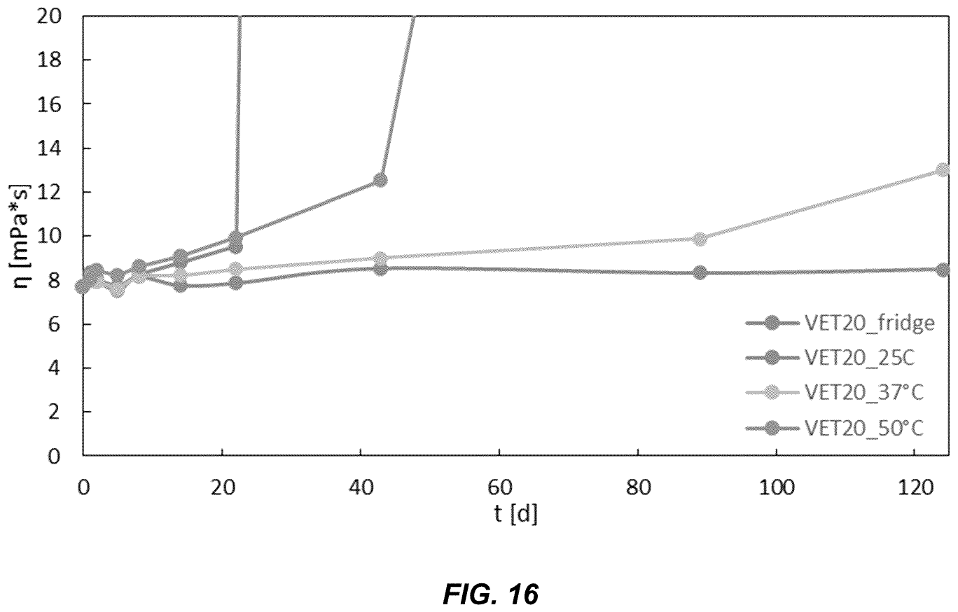

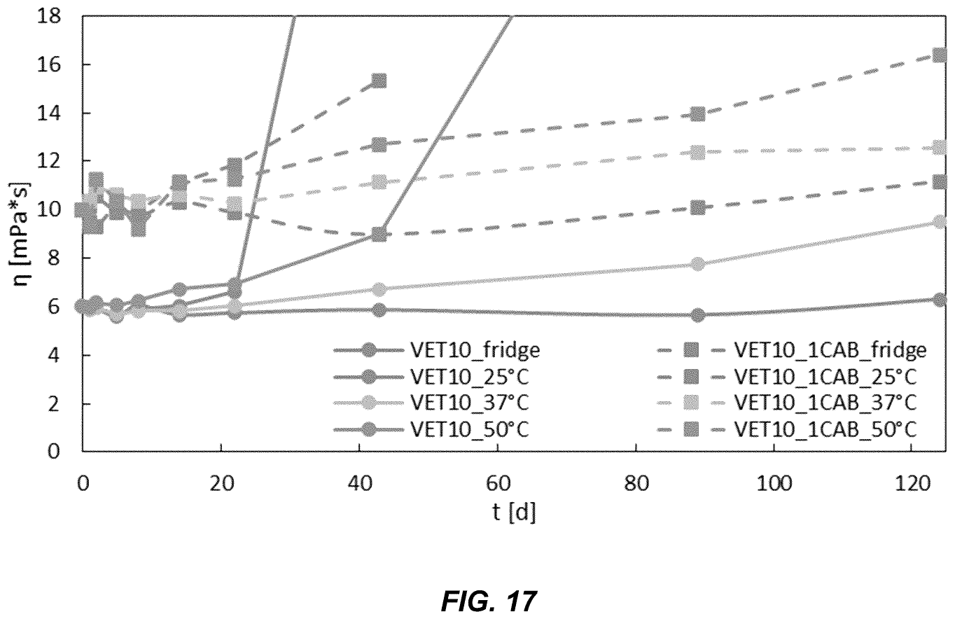

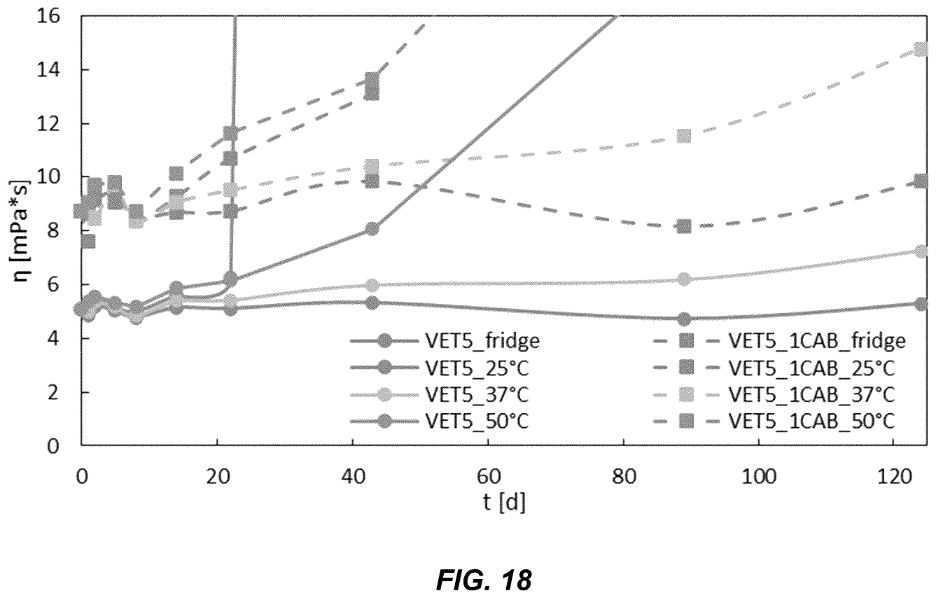

The materials were also investigated with laser scanning microscopy (LSM). FIG. 15 provides the side view (top row) and top view (bottom row) of the digital materials with layers (left column), lines (middle column), and dots (right column) of the printed curable composition. The side view of the curable composition layer-containing material (top left image) evidences uneven curable composition distribution, with some curable composition missing and thicker lower layers. While these may have been due to the nozzles and sample preparation, they may relate to the relatively lower tensile strength measured for this digital material. For the curable composition line-containing material, the curable composition lines are visible in the side view, while the top view suggests that not all lines shared identical axes. The top and side views of the curable composition dot-containing material suggest good evenness and dot separation with this printing technique.

Holistically, these results demonstrate that the curable composition showed excellent polymerization behavior with minimal inhibition by oxygen. The curable composition was shown to have a low viscosity, low glass transition temperature, modulus, and elastomeric behavior of the curable composition suggests that it is easy to handle and process. It was further shown that sufficient stability could be achieved with high stabilizer concentrations. From these results, it is postulated that decreasing the cross-linker content could further improve the elongation at break and toughness of the curable composition.

### Example 6

### Digital Material Tensile Strength

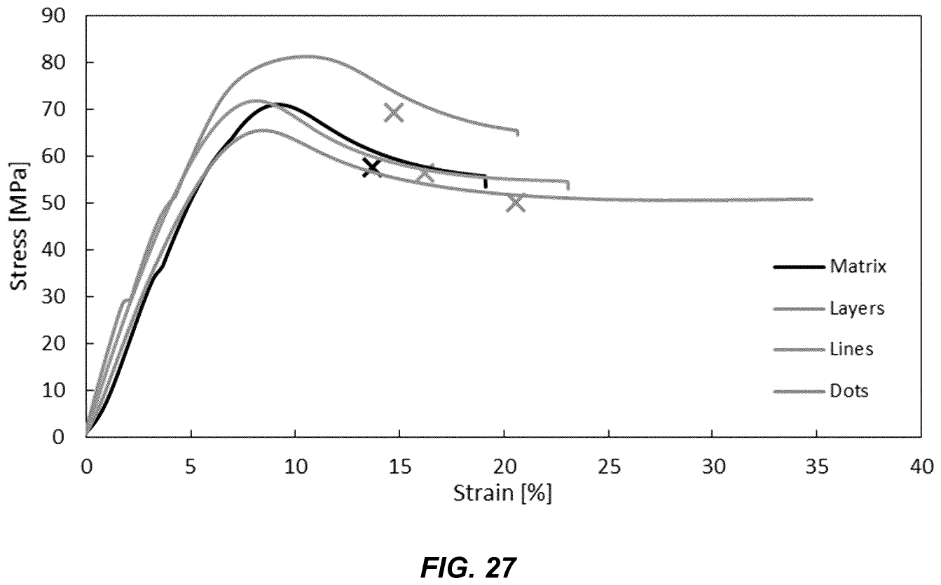

This example covers fabrication and tensile strength testing of a digital material printed with layers of a polymeric matrix material interspersed by dots, lines, or layers of a curable composition. The matrix material used for the digital materials contained 60 wt % of Bomar XR-741MS, 15 wt % 2-hydroxyethyl methacrylate (HEMA), and 25 wt % homosalate methacrylate (HSMA). The curable composition was VET20 as described in EXAMPLE 2. Different printing speeds and light pulse durations were tested, again applying the curable composition VET20 via inkjet printer between the 50 μm matrix layers in form of layers, lines and dots. Optimal results in terms of toughening were received with a printing speed of 20 mm/s and a light pulse duration of 7000. Tensile test curves of the matrix and digital materials with different curable composition geometries were tested for systems generated with these parameters. Results of these analyses are provided in TABLE 7 and FIG. 27.

TABLE 7

| | ε_{B} | Δε_{B} | σ_{M} | Δσ_{M} | Toughness | ΔToughness |
|---|---|---|---|---|---|---|
| Sample | [%] | [% ] | [Mpa] | [%] | [MJ/m³] | [%] |
| MATRIX | 13.7 ± 2.9 | — | 67.9 ± 3.1 | — | 6.8 ± 1.7 | — |
| MATRIX WITH | 20.5 ± 6.1 | 50.0 | 64.7 ± 3.0 | −4.7 | 10 ± 3.3 | 46.6 |
| VET20 LAYERS | | | | | | |
| MATRIX WITH | 16.2 ± 3.2 | 18.0 | 71.6 ± 1.3 | 5.5 | 8.6 ± 1.9 | 26.7 |
| VET20 LINES | | | | | | |
| MATRIX WITH | 14.7 ± 2.5 | 7.4 | 83.2 ± 1.5 | 22.6 | 9.1 ± 1.8 | 33.2 |
| VET20 DOTS | | | | | | |

The matrix material reached a maximum elongation at break of 19.1% and exhibited an average elongation break of 13.7%. The addition of the curable composition VET20 increased elongation at break for all tested curable composition geometries (dots, lines, and layers) in terms of maximum and average measured values. For the tested materials, increasing the amount of curable composition increased elongation at break and lowered tensile strength. However, the average tensile strength of the digital materials (containing the matrix material and curable composition) were higher than that of the pure matrix material with the exception of the layered-ink materials, which exhibited slightly decreased tensile strengths. Nevertheless, all digital materials exhibited significant toughness increases, likely attributable either to increased elongation at break or to increase of elongation at break and tensile strength. The highest toughness was achieved with the curable composition layer geometry, followed by the dot geometry, and finally the line geometry.

### Example 7

### Digital Material Tensile Strength

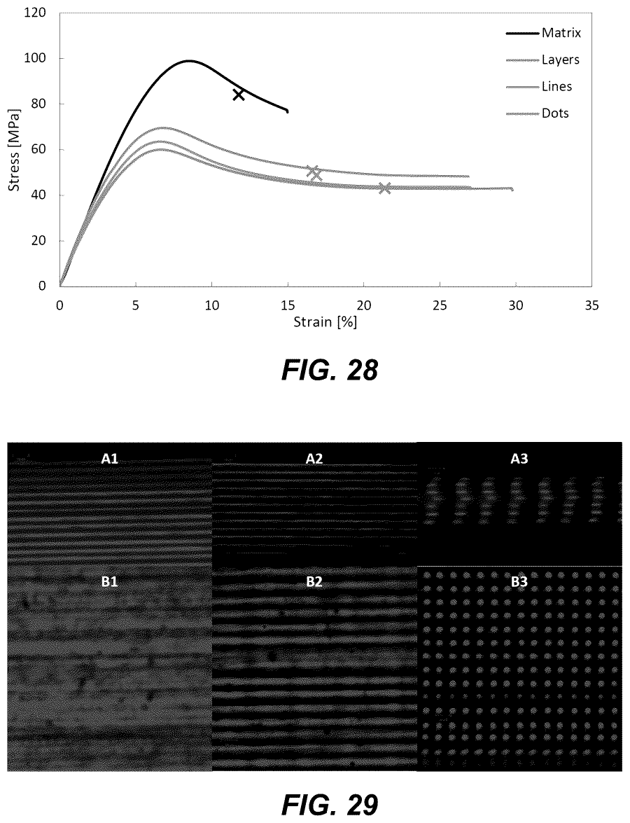

This example covers fabrication and tensile strength testing of a digital material printed with layers of a polymeric matrix material interspersed by dots, lines, or layers of a curable composition. The matrix material used for the digital materials contained 60 wt % of Bomar XR-741MS, 15 wt % homosalate methacrylate (HSMA), and 25 wt % 2-hydroxyethyl methacrylate (HEMA). The curable composition was VET5 as described in EXAMPLE 2. Different printing speeds and light pulse durations were tested, again applying the curable composition VET5 via inkjet printer between the 50 μm matrix layers in form of layers, lines and dots. Optimal results in terms of toughening were received with a printing speed of 20 mm/s and a light pulse duration of 7000. Tensile test curves of the matrix and digital materials with different curable composition geometries were tested for systems generated with these parameters. Results of these analyses are provided in TABLE 8 and FIG. 28.

TABLE 8

| | ε_{B} | Δε_{B} | σ_{M} | Δσ_{M} | Toughness | ΔToughness |
|---|---|---|---|---|---|---|
| Sample | [%] | [%] | [Mpa] | [%] | [MJ/m³] | [%] |
| MATRIX | 11.8 ± 1.5 | — | 98.2 ± 3.4 | — | 8.1 ± 1.3 | — |
| MATRIX WITH | 21.4 ± 4.6 | 81.5 | 61.2 ± 2 | −37.6 | 9.8 ± 1.9 | 21.4 |
| VET5 LAYERS | | | | | | |
| MATRIX WITH | 16.9 ± 4.2 | 43.4 | 66 ± 2.3 | −32.8 | 8.4 ± 2 | 4.3 |
| VET5 LINES | | | | | | |
| MATRIX WITH | 16.6 ± 5.3 | 41.1 | 69.4 ± 1.1 | −29.3 | 8.7 ± 2.8 | 8.0 |
| VET5 DOTS | | | | | | |

The matrix material reached a maximum elongation at break of 15% and exhibited an average elongation break of 11.8%. The addition of the curable composition VET5 increased elongation at break for all tested curable composition geometries (dots, lines, and layers) in terms of maximum and average measured values. However, the average tensile strength of the digital materials (containing the matrix material and curable composition) were lower than that of the pure matrix material. Nevertheless, all digital materials exhibited toughness increases, likely attributable to increased elongation at break. The highest toughness was achieved with the curable composition layer geometry, followed by the dot geometry, and finally the line geometry.

The digital materials were also investigated with LSM. FIG. 29 provides the side view (top row) with a magnification of 10 and a zoom of 0.5 and top view (bottom row) with a magnification of 2.5 of the digital materials with layers (left column), lines (middle column), and dots (right column) of the printed curable composition. The side view of the curable composition layer-containing material (top left image) evidences uneven curable composition distribution, with thicker lower layers. While these may have been due to the nozzles and sample preparation, they may relate to the relatively lower tensile strength measured for this digital material. For the curable composition line-containing material, the curable composition lines are visible in the side view, while the top view suggests that not all lines shared identical axes. The top and side views of the curable composition dot-containing material suggest good evenness and dot separation with this printing technique.

### Example 8

### Digital Material Tensile Strength

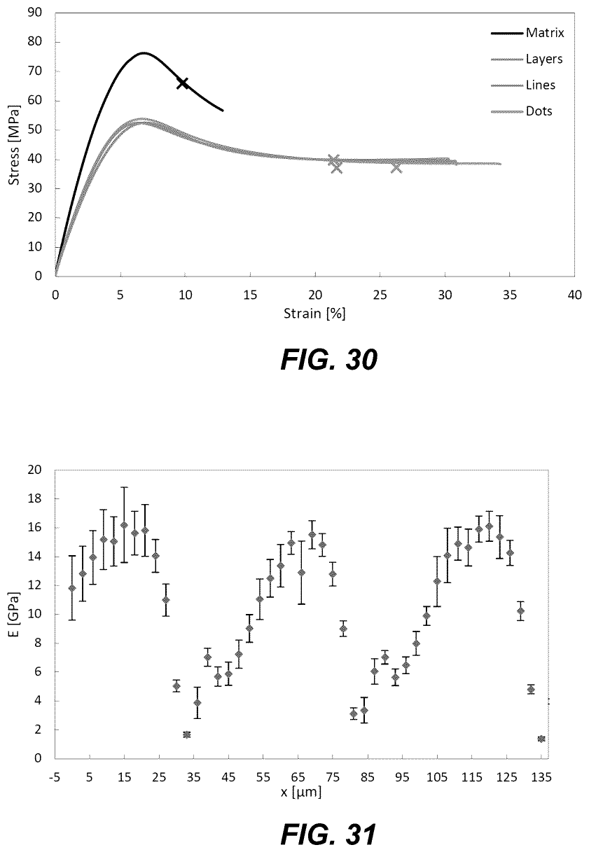

This example covers fabrication and tensile strength testing of a digital material printed with layers of a polymeric matrix material interspersed by dots, lines, or layers of a curable composition. The matrix material used for the digital materials contained 60 wt % of Bomar XR-741MS, 15 wt % homosalate methacrylate (HSMA), and 25 wt % 2-hydroxyethyl methacrylate (HEMA). The curable composition was VET10 as described in EXAMPLE 2. Different printing speeds and light pulse durations were tested, again applying the curable composition VET10 via inkjet printer between the 50 μm matrix layers in form of layers, lines and dots. Optimal results in terms of toughening were received with a printing speed of 20 mm/s and a light pulse duration of 7000. Tensile test curves of the matrix and digital materials with different curable composition geometries were tested for systems generated with these parameters. Results of these analyses are provided in TABLE 9 and FIG. 30.

TABLE 9

| | ε_{B} | Δε_{B} | σ_{M} | Δσ_{M} | Toughness | ΔToughness |
|---|---|---|---|---|---|---|
| Sample | [%] | [%] | [Mpa] | [%] | [MJ/m³] | [%] |
| MATRIX | 9.8 ± 1.9 | — | 76 ± 3.4 | — | 5.4 ± 1.3 | — |
| MATRIX WITH | 21.7 ± 5.1 | 121.8 | 49.5 ± 4.9 | −34.9 | 8.4 ± 2.3 | 56.0 |
| VET10 LAYERS | | | | | | |
| MATRIX WITH | 26.3 ± 6.4 | 168.9 | 50.9 ± 2.2 | −33.1 | 10.3 ± 2.4 | 90.7 |
| VET10 LINES | | | | | | |
| MATRIX WITH | 21.5 ± 6.7 | 119.6 | 51.4 ± 3.2 | −32.4 | 8.6 ± 2.4 | 59.4 |
| VET10 DOTS | | | | | | |

The matrix material reached a maximum elongation at break of 13% and exhibited an average elongation break of 9.8%. The addition of the curable composition VET10 increased elongation at break for all tested curable composition geometries (dots, lines, and layers) in terms of maximum and average measured values. However, the average tensile strength of the digital materials (containing the matrix material and curable composition) were lower than that of the pure matrix material. Nevertheless, all digital materials exhibited significant toughness increases, likely attributable to increased elongation at break. The highest toughness was achieved with the curable composition line geometry, followed by the dot geometry, and finally the layer geometry.

The average E-modulus were calculated for the digital material with the curable composition in layer geometry, and the results are shown in FIG. 31. The layers exhibited visible minima in E-modulus and hardness every 40 to 50 μm, corresponding approximately to the target layer thickness of 50 μm.

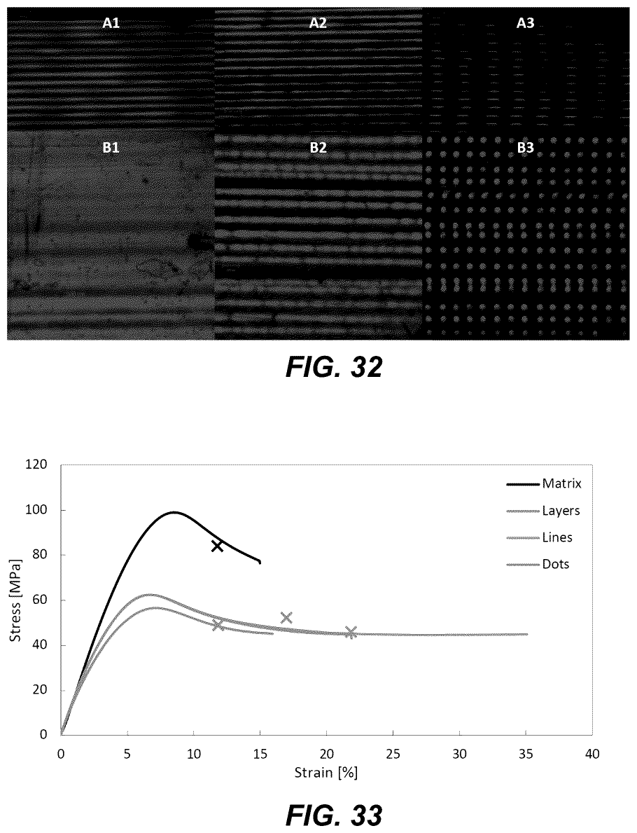

The digital materials were also investigated with laser scanning microscopy (LSM). FIG. 32 provides the side view (top row) with a magnification of 10 and a zoom of 0.5 and top view (bottom row) with a magnification of 2.5 of the digital materials with layers (left column), lines (middle column), and dots (right column) of the printed curable composition. The side view of the curable composition layer-containing material (top left image) evidences even curable composition distribution. For the curable composition line-containing material, the curable composition lines are visible in the side view, while the top view suggests that not all lines shared identical axes. The top and side views of the curable composition dot-containing material suggest good evenness and dot separation with this printing technique.

### Example 9

### Digital Material Tensile Strength

This example covers fabrication and tensile strength testing of a digital material printed with layers of a polymeric matrix material interspersed by dots, lines, or layers of a curable composition. The matrix material used for the digital materials contained 60 wt % of Bomar XR-741MS, 15 wt % homosalate methacrylate (HSMA), and 25 wt % 2-hydroxyethyl methacrylate (HEMA). The curable composition was VET20 as described in EXAMPLE 2. Different printing speeds and light pulse durations were tested, again applying the curable composition VET20 via inkjet printer between the 50 μm matrix layers in form of layers, lines and dots. Optimal results in terms of toughening were received with a printing speed of 20 mm/s and a light pulse duration of 7000. Tensile test curves of the matrix and digital materials with different curable composition geometries were tested for systems generated with these parameters. Results of these analyses are provided in TABLE 10 and FIG. 33.

TABLE 10

| | ε_{B} | Δε_{B} | σ_{M} | Δσ_{M} | Toughness | ΔToughness |
|---|---|---|---|---|---|---|
| Sample | [%] | [%] | [Mpa] | [%] | [MJ/m³] | [%] |
| MATRIX | 11.8 ± 1.5 | — | 98.2 ± 3.4 | — | 8.1 ± 1.3 | — |
| MATRIX WITH | 11.9 ± 2.6 | 0.6 | 59.2 ± 4.4 | −39.7 | 5.4 ± 1.4 | −33.7 |
| VET20 LAYERS | | | | | | |
| MATRIX WITH | 21.9 ± 6.6 | 85.5 | 62.8 ± 1.2 | −36.0 | 10.4 ± 3 | 28.7 |
| VET20 LINES | | | | | | |
| MATRIX WITH | 17 ± 6.6 | 44.2 | 67.2 ± 4.3 | −31.5 | 8.6 ± 3.3 | 6.1 |
| VET20 DOTS | | | | | | |

The matrix material reached a maximum elongation at break of 15% and exhibited an average elongation break of 11.8%. The addition of the curable composition VET20 increased elongation at break for the tested curable composition in dot and line geometries in terms of maximum and average measured values. However, for the tested curable composition in layer geometry, the addition of the curable VET20 only slightly increased the maximum elongation at break value but had little effect on the average elongation at break value. The average tensile strength of the digital materials (containing the matrix material and curable composition) were lower than that of the pure matrix material. Only digital materials with the curable composition in dot and line geometries exhibited toughness increases, likely attributable to increased elongation at break. The digital material with the curable composition in layer geometry exhibited toughness decreases. The highest toughness was achieved with the curable composition in line geometry, followed by the dot geometry, and finally the layer geometry.

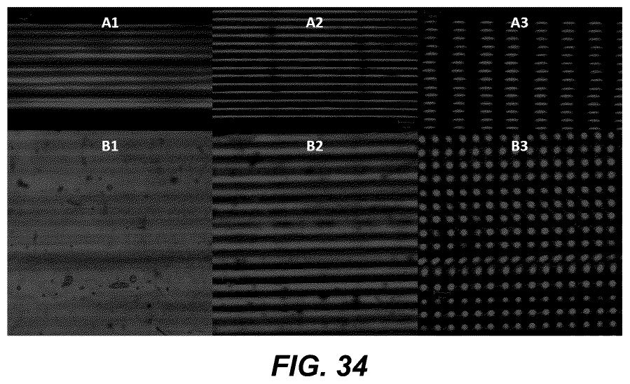

The digital materials were also investigated with laser scanning microscopy (LSM). FIG. 34 provides the side view (top row) with a magnification of 10 and a zoom of 0.5 and top view (bottom row) with a magnification of 2.5 of the digital materials with layers (left column), lines (middle column), and dots (right column) of the printed curable composition. The side view of the curable composition layer-containing material (top left image) evidences uneven curable composition distribution, with thicker lower layers. For the curable composition line-containing material, the op and side views show good line evenness. The top and side views of the curable composition dot-containing material suggest good evenness and dot separation with this printing technique.

### Example 10

### Treatment Using an Orthodontic Appliance

This example describes the use of a directly 3D printed orthodontic appliance to move a patient's teeth according to a treatment plan. This example also describes the characteristics that the orthodontic appliance can have following its use, in contrast to its characteristics prior to use.

A patient in need of, or desirous of, a therapeutic treatment to rearrange at least one tooth has their teeth arrangement assessed. An orthodontic treatment plan is generated for the patient. The orthodontic treatment plan comprises a plurality of intermediate tooth arrangements for moving teeth along a treatment path, from the initial arrangement (e.g., that which was initially assessed) toward a final arrangement. The treatment plan includes the use of an orthodontic appliance, fabricated using the compositions and methods disclosed further herein, to provide orthodontic appliances having a plurality of polymer phases. In some embodiments, a plurality of orthodontic appliances are used, each of which can be fabricated using the compositions and methods disclosed further herein.

The orthodontic appliances are provided, and iteratively applied to the patient's teeth to move the teeth through each of the intermediate tooth arrangements toward the final arrangement. The patient's tooth movement is tracked. A comparison is made between the patient's actual teeth arrangement and the planned intermediate arrangement. Where the patient's teeth are determined to be tracking according to the treatment plan, but have not yet reached the final arrangement, the next set of appliances can be administered to the patient. The threshold difference values of a planned position of teeth to actual positions selected as indicating that a patient's teeth have progressed on-track are provided above in Table 11. If a patient's teeth have progressed at or within the threshold values, the progress is considered to be on-track. Favorably, the use of the appliances disclosed herein increases the probability of on-track tooth movement.

TABLE 11

| Type Movement | Difference Actual/Planned |
|---|---|
| Rotations | |
| Upper Central Incisors | 9 degrees |
| Upper Lateral Incisors | 11 degrees |
| Lower Incisors | 11 degrees |
| Upper Cuspids | 11 degrees |
| Lower Cuspids | 9.25 degrees |
| Upper Bicuspids | 7.25 degrees |
| Lower First Bicuspid | 7.25 degrees |
| Lower Second Bicuspid | 7.25 degrees |
| Molars | 6 degrees |
| Extrusion | |
| Anterior | 0.75 mm |
| Posterior | 0.75 mm |
| Intrusion | |
| Anterior | 0.75 mm |
| Posterior | 0.75 mm |
| Angulation | |
| Anterior | 5.5 degrees |
| Posterior | 3.7 degrees |
| Inclination | |
| Anterior | 5.5 degrees |
| Posterior | 3.7 degrees |
| Translation | |
| BL Anterior | 0.7 mm |
| BL Posterior Cuspids | 0.9 mm |
| MD Anterior | 0.45 mm |
| MD Cuspids | 0.45 mm |
| MD Posterior | 0.5 mm |

The assessment and determination of whether treatment is on-track can be conducted, for example, 1 week (7 days) following the initial application of an orthodontic appliance. Following this period of application, additional parameters relating to assessing the durability of the orthodontic appliance can also be conducted. For example, relative repositioning force (compared to that which was initially provided by the appliance), intactness of polymer chains (e.g., the percent of polymer chains that are not broken), relative flexural modulus, and relative elongation at break can be determined.

1. A method of producing a multilayered article, the method comprising:
producing a first layer of the multilayered article by photolithography from a matrix material; and
producing a second layer of the multilayered article on the first layer by inkjet printing using an ink through a thiol-ene reaction.

2. The method of claim 1,
wherein producing the first layer of the multilayered article comprises curing a first portion of a matrix material having a first composition; and
wherein producing the second layer of the multilayered article comprises:
applying an ink that is stable for at least 20 days at 25° C. having a second composition to the first layer of the multilayered article; and
curing the ink.

3. The method of claim 2, further comprising separating the first layer of the multilayered article from an uncured portion of the matrix material.

4.-5. (canceled)

6. The method of claim 1, wherein the ink has a viscosity of between about 3 mPa*s and about 50 mPa*s.

7.-10. (canceled)

11. The method of claim 1, further comprising repeating the method to form a plurality of the first and the second layers of the multilayered article.

12. (canceled)

13. The method of claim 1, wherein the first layer and the second layer each comprises a thickness of between 5 and 250 microns (μm).

14. The method of claim 1, wherein the second layer is fully contiguous with the first layer or partially contiguous with respect to the first layer.

15. (canceled)

16. The method of claim 14, wherein the second layer comprises a structure selected from the group consisting of a dot, a linear segment, a continuous layer, or a combination thereof.

17. (canceled)

18. The method of claim 1, wherein the matrix comprises an acrylate or a methacrylate, and the ink comprises a vinyl ether and a thiol.

19. The method of claim 2, wherein the curing the first composition is performed by stereolithography.

20. The method of claim 3, wherein the separating the first layer of the multilayered article comprises removing the multilayered article or a portion of the multilayered article from a vat comprising the uncured portion of the matrix material.

21. The method of claim 2, wherein the curing the matrix material having the first composition comprises using a different central wavelength of light than curing the ink having the second composition.

22.-23. (canceled)

24. The method claim 18, wherein the matrix material and the ink independently further comprises:
(a) a crosslinking agent,
(b) a photoinitiator,
(c) a stabilizer, the stabilizer comprising a radical scavenger, an oxygen scavenger, or a combination thereof.
(d) a filler,
(e) a dye, or
(f) any combination of (a)-(e) thereof.

25.-27. (canceled)

28. The method of claim 24, wherein the vinyl ether comprises tri(ethylene glycol) divinyl ether (TEGDVE), the thiol comprises 2,2′-(ethylenedioxy diethanethiol (EDDT), and the crosslinking agent comprises 1,1,1-tris-(hydroxymethyl)-propane-tri s-(3-mercaptopropionate) TMPMP.

29. (canceled)

30. The method claim 2, wherein curing the ink having the second composition reaches at least 95% completion after at most 10 seconds.

31. The method of claim 1, wherein the ink comprises:
(a) a viscosity of between 3 and 50 mPa*s at 25° C.;
(b) a final density of between 0.9 and 1.5 g/cm³ at 25° C.;
(c) an Ohnesorge number of between 0.1 and 1;
(d) a glass transition temperature of between −70° C. and −35° C.;
(e) a storage modulus of between 0.25 and 5.0 MPa;
a surface tension of between 35 and 40.6 mN/m at 24° C.; or
(g) any combination of (a)-(e) thereof.

32. The method of claim 1, wherein the multilayered article comprises:
(a) an elastic modulus of between 8 and 20 GPa;
(b) an elongation at break of between 10% and 45%;
(c) a toughness of between 0.3 and 21 MJ/m³; or
(d) any combination of (a)-(c) thereof.

33. The method claim 1, wherein the ink comprises:
(a) a first difunctional monomer comprising two ethylenically unsaturated functional groups;
(b) a second difunctional monomer comprising two thiol functional groups; and
(c) a crosslinker comprising at least three thiol or at least three ethylenically unsaturated functional groups;
wherein a molar ratio of ethylenically unsaturated functional groups and thiol groups is between 2:5 to 5:2.

34. The method of claim 1, wherein the ink comprises a viscosity of between 3 and 50 mPa*s.

35. The method of claim 1, wherein the ink increases in viscosity by no more than 80% when stored at 25° C. for 20 days in the absence of light.

36. The method of claim 33, wherein the two ethylenically unsaturated functional groups are vinyl ether groups.

37. (canceled)

38. The method of claim 2, wherein a 200 μm thick film of the second composition is configured to cure at a rate of at least 10%/s.

39. The method of claim 38, wherein the curing the ink comprises photocuring the 200 μm thick film of the photocurable composition under 14 mW/cm² light at a wavelength for activating a photoinitiator of the second composition.

40. The method of claim 33, wherein the second composition comprises between 6.8 and 25.4 w % of the crosslinker.

41. The method of claim 33, wherein the crosslinker comprises 3 polymerizable thiol groups or at least 3 ethylenically unsaturated functional groups.

42. The method of claim 33, wherein the first difunctional monomer, the second difunctional monomer, or both the first difunctional monomer and the second difunctional monomer comprise glycol.

43. The method of claim 42, wherein the glycol is disposed in a backbone of the first difunctional monomer, a backbone of the second difunctional monomer, or the backbones of both the first difunctional monomer and the second difunctional monomer.

44. The method of claim 33, wherein the ink further comprises cellulose acetate butyrate.

45. A multilayered article produced according to the method of claim 1.

46. A medical device comprising the multilayered article of claim 45.

47.-58. (canceled)

59. A digital multilayered article, comprising:
a first layer comprising an acrylate or a methacrylate; and
a second layer chemically bonded to the first layer and cured from an ink material selected from the group consisting of tri(ethylene glycol) divinyl ether (TEGDVE) and 2,2′-(ethylenedioxy diethanethiol (EDDT), a crosslinking agent comprising 1,1,1-tris-(hydroxymethyl)-propane-tris-(3-mercaptopropionate) (TMPMP).

60.-70. (canceled)

71. The medical device of claim 46, wherein the medical device is an orthodontic appliance.

72. The medical device of claim 71, wherein the orthodontic appliance is a dental aligner.

73. A method of repositioning a patient's teeth, the method comprising:
generating a treatment plan for the patient, the plan comprising a plurality of intermediate tooth arrangements for moving teeth along a treatment path from an initial tooth arrangement toward a final tooth arrangement;
producing an orthodontic appliance of claim 71; and
moving on-track, with the orthodontic appliance, at least one of the patient's teeth toward an intermediate tooth arrangement or the final tooth arrangement.

74.-78. (canceled)
